(19) United States
(12) Patent Application Publication
(10) Pub. No.: US 2011/0166156 A1
(43) Pub. Date: Jul. 7, 2011

(54) Prodrugs for the Treatment of Schizophrenia and Bipolar Disease

(75) Inventors: Laura Cook Blumberg, Lincoln, MA (US); Örn Almarsson, Shrewsbury, MA (US)

(73) Assignee: Alkermes, Inc., Waltham, MA (US)

(21) Appl. No.: 12/978,274

(22) Filed: Dec. 23, 2010

Related U.S. Application Data
(60) Provisional application No. 61/293,163, filed on Jan. 7, 2010.
(60) Provisional application No. 61/293,153, filed on Jan. 7, 2010.

(51) Int. Cl. A61K 31/496; C07D 239/70; A61K 31/519; C07D 417/14; C07D 413/04; A61K 31/454; A61P 25/18

(52) U.S. Cl. 514/254.04; 544/282; 514/259.41; 544/368; 546/198; 514/321

(57) ABSTRACT

Compounds of Formula I and Formula II and their use for the treatment of neurological and psychiatric disorders including schizophrenia and manic or mixed episodes associated with bipolar I disorder with or without psychotic features is disclosed.

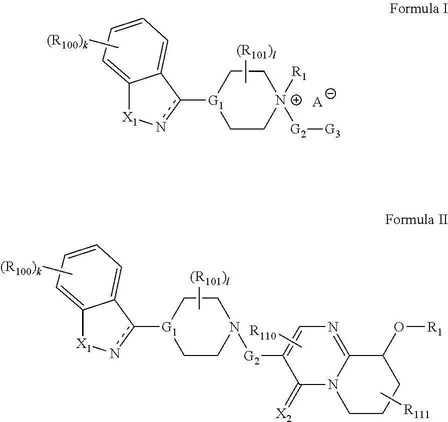

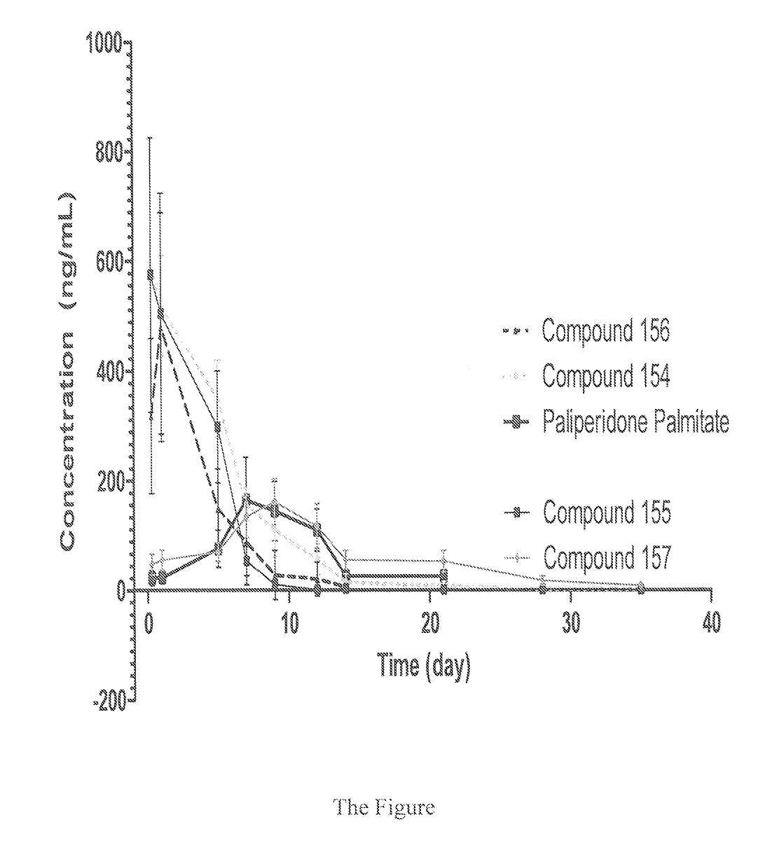

### RELATED APPLICATION(S)

This application claims the benefit of U.S. Provisional Application Nos. 61/293,163 and 61/293,153, both filed on Jan. 7, 2010. The entire teachings of the above application(s) are incorporated herein by reference.

### BACKGROUND OF THE INVENTION

Paliperidone, risperidone, iloperidone, lurasidone and ziprasidone are atypical antipsychotic drugs, all of which are approved by the U.S. Food and Drug Administration for the treatment of schizophrenia and bipolar mania. Two additional atypical antipsychotic drugs, perphenazine GABA ester (BL-1020) and perospirone have shown potential for treatment of schizophrenia and bipolar mania. The chemical structures of these heterocyclic compounds are given below.

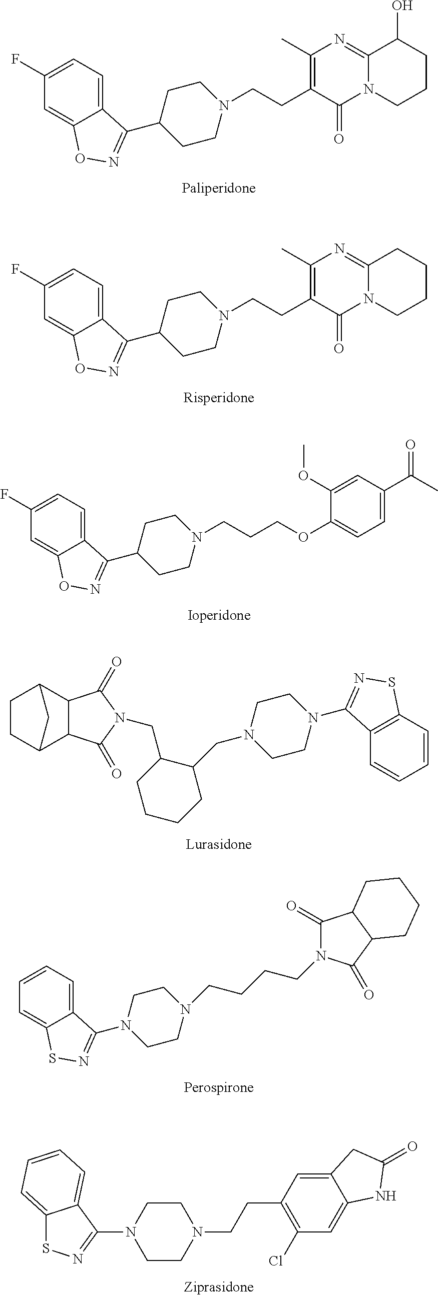

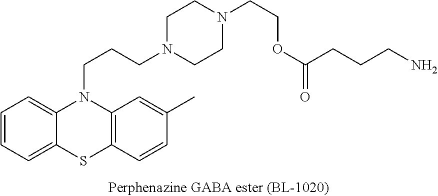

Other examples of heterocyclic derivatives that are useful for the treatment of schizophrenia and bipolar disorders are discussed in U.S. Pat. No. 5,350,747, U.S. Pat. No. 5,006,528, U.S. Pat. No. 7,160,888, and in U.S. Pat. No. 6,127,357. Heterocyclic derivatives that have been stated to be useful as antipsychotic agents are discussed in WO 93/04684 and European patent application EP 402644. INVEGA® SUSTENNA® is a paliperidone-palmitate ester conjugate used as a long-acting atypical antipsychotic. Kramer et. al., International Journal of Neuropsycho-Pharmacology, 2009, 1-13; Citrome L., Patient Preference and Adherence, 2009 (3); 345-355.

Drug delivery systems are often critical for the safe and effective administration of a biologically active agent. Perhaps the importance of these systems is best realized when drug bioavailability, patient compliance, and consistent dosing are taken under consideration. For instance, reducing the dosing requirement for a drug from four-times-a-day to a single dose per day, or to once a week or even less frequently would have significant value in terms of ensuring patient compliance.

In an attempt to address the need for improved bioavailability several drug release modulation technologies have been developed. Enteric coatings have been used as a protector of pharmaceuticals in the stomach and microencapsulating active agents using protenoid microspheres, liposomes or polysaccharides have been effective in abating enzyme degradation of the active agent. Enzyme inhibiting adjuvants have also been used to prevent enzyme degradation.

While microencapsulation and enteric coating technologies impart enhanced stability and time-release properties to active agent substances, these technologies suffer from several shortcomings. Incorporation of the active agent is often dependent on diffusion into the microencapsulating matrix, which may not be quantitative and may complicate dosage reproducibility. In addition, encapsulated drugs rely on diffusion out of the matrix or degradation of the matrix, which is highly dependent on the water solubility and partitioning properties of the active agent. Conversely, water-soluble microspheres swell by an infinite degree and, unfortunately, may release the active agent in bursts with little active agent remaining available for sustained release. Additionally, there is a need for an active agent delivery system that is able to deliver certain active agents which have been heretofore not formulated or difficult to formulate in a sustained release formulation, and which is convenient for patient dosing.

### SUMMARY OF THE INVENTION

The instant application relates to compounds of Formula I and their use for the treatment of neurological and psychiatric disorders including schizophrenia and bipolar disease. In particular, the instant application relates to compounds of formula I and II:

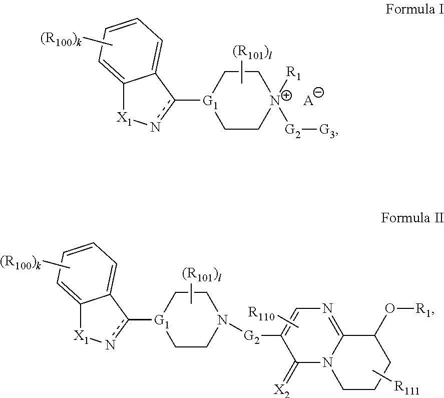

or the geometric isomers, enantiomers, diastereomers, racemates, pharmaceutically acceptable salts, co-crystals or solvates thereof;
wherein [symbol] represents a single or double bond;
each k and l is independently 0, 1, 2, 3, or 4;
A⁻ is a pharmaceutically acceptable anion;

### X₁ is —CR₁₀—, —O— or —S—;

wherein each R₁₀ is independently hydrogen, halogen, aliphatic, substituted aliphatic, aryl or substituted aryl;

### X₂ is 0 or 5;

### G₁ is —N— or —CR₁₀—;

G₂ is selected from absent, —C(O)(C(R₁₀)(R₁₁))ₜ—, —C(R₁₀)═C(R₁₁)—, —(C(R₁₀)(R₁₁))ₐ═(C(R₁₀)(R₁₁)_{b}—, —(C(R₁₀)(R₁₁))ₐ—X₁₀—(C(R₁₀)(R₁₁)_{b}—, and —(C(R₁₀)(R₁₁))ₜ—;
wherein t is 1, 2, 3, 4, 5 or 6;
each a and b is independently 0, 1, 2, 3, 4, 5, 6, 7, 8, 9 or 10;
each R₁₁ is independently hydrogen, halogen, aliphatic, substituted aliphatic, aryl or substituted aryl;
X₁₀ is absent, cycloalkyl, —S—, —O—, —N(R₁₀)—, —C(O)—, —C(S)—, —C(R₁₀)═C(R₁₀)—, or —C≡C—;
alternatively, two R₁₀ and R₁₁ groups together with the atoms they are attached form a three, four, five or six membered ring;
   G₃ is an optionally substituted cyloalkyl or optionally substituted heterocyclyl;
   R₁ is selected from —C(R₁₀)(R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(OR₂₁), —C(R₁₀)(R₁₁)—OP(O)(OR₂₁(OR₂₂);
each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
   each R₁₀₀, R₁₀₁, R₁₁₀ and R₁₁₁ is independently selected from hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ alkylamino and optionally substituted C₁-C₈ aryl; and
   Y and M are the same or different and each is a monovalent cation;
   or M and Y together is a divalent cation.

The prodrug compounds of the invention incorporate a labile prodrug moiety which is cleaved in vivo to produce a bioactive compound such as paliperidone, risperidone, iloperidone, perospirone, lurasidone, or ziprasidone. Paliperidone, risperidone, iloperidone, lurasidone, perospirone, and ziprasidone are parent drugs from which prodrugs of the invention are derived that are useful in the treatment of schizophrenia and bipolar disorder. The addition of the prodrug moiety allows modification of the physical properties of these the parent drugs providing extended-release formulations. While a specific isomeric form of a parent drug may be preferred for use in treatment, the term “parent drug” as used herein is intended to encompass all isomers of the parent drug. It is also understood that the parent drug may be further “substituted” as that term is defined herein, for any purpose including but not limited to, stabilization of the parent during synthesis of the prodrug and stabilization of the prodrug for administration to the patient. One example of a substituted parent drug is a pharmaceutically acceptable ester of the parent drug. Any of the parent drugs and prodrugs of parent drugs of the invention may be substituted so long as the substituted parent drug or parent prodrug when administered to a patient in vivo becomes cleaved by chemical and/or enzymatic hydrolysis thereby releasing the parent drug moiety such that a sufficient amount of the compound intended to be delivered to the patient is available for its intended therapeutic use in a sustained release manner.

### BRIEF DESCRIPTION OF THE DRAWING

The FIGURE is a line graph of showing the combined results derived from two separate pharmacokinetic studies in a rat wherein the compounds tested were paliperidone prodrug compounds.

### DETAILED DESCRIPTION OF THE INVENTION

The addition of aldehyde-linked hydrophobic and/or lipophilic prodrug moieties to a piperidine or piperazine nitrogen atoms in certain parent drug compounds, such as paliperidone, risperidone, iloperidone, perospirone, lurasidone, and ziprasidone, results in labile prodrugs which have reduced solubility and polarity compared to the parent drug and therefore are useful in extended release formulations. In addition, embodiments in which the prodrug moiety comprises a phosphonate group, modification of the phosphonate group, through esterification with lipophilic groups, will modulate the solubility of the prodrugs. The physical chemical and solubility properties of these derivatives can be further modulated by the choice of counterion A⁻ (i.e. Cl⁻, Br⁻, I⁻, CH₃CO₂⁻ or other organic anion).

The parent drug, such as paliperidone, risperidone, iloperidone, perospirone, lurasidone, and ziprasidone, will be released from such prodrugs by enzymatic and/or chemical cleavage in vivo, thereby releasing the original tertiary amine-containing parent drug.

One aspect of the present invention provides a compound having the general formula I and II:

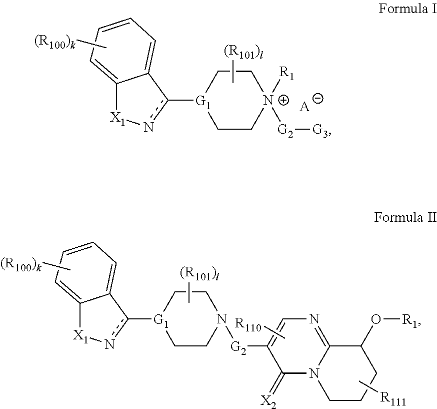

or the geometric isomers, enantiomers, diastereomers, racemates, pharmaceutically acceptable salts or solvates thereof;
wherein [symbol] represents a single or double bond;
each k and l is independently 0, 1, 2, 3, or 4;
A⁻ is a pharmaceutically acceptable anion;

### X₁ is —CR₁₀—, —O— or —S—;

wherein each R₁₀ is independently hydrogen, halogen, aliphatic, substituted aliphatic, aryl or substituted aryl;

### X₂ is O or S;

### G₁ is —N— or —CR₁₀—;

G₂ is selected from absent, —C(O)(C(R₁₀)(R₁₁))ₜ—, —C(R₁₀)═C(R₁₁)—, —(C(R₁₀)(R₁₁))=(C(R₁₀)(R₁₁)_{b}—, —(C(R₁₀)(R₁₁))ₐ—X₁₀—(C(R₁₀)(R₁₁)_{b}— and, —(C(R₁₀)(R₁₁))ₜ—;
wherein t is 1, 2, 3, 4, 5 or 6;
each a and b is independently 0, 1, 2, 3, 4, 5, 6, 7, 8, 9 or 10;
each R₁₁ is independently hydrogen, halogen, aliphatic, substituted aliphatic, aryl or substituted aryl;
X₁₀ is absent, cylcoalkyl, —S—, —O—, —N(R₁₀)—, —C(O)—, —C(S)—, —C(R₁₀)═C(R₁₀)—, or —C≡C—;
alternatively two R₁₀ and R₁₁ groups together with the atoms they are attached form a three, four, five or six membered ring;
   G₃ is an optionally substituted cyloalkyl or optionally substituted heterocyclyl;
   R₁ is selected from —C(R₁₀)(R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(OR₂₁), —C(R₁₀)(R₁₁)—OP(O)(OR₂₁)(OR₂₂);
each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
   each R₁₀₀, R₁₀₁, R₁₁₀ and R₁₁₁ is independently selected from hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ alkylamino and optionally substituted C₁-C₈ aryl; and
   Y and M are the same or different and each is a monovalent cation;
   or M and Y together is a divalent cation. Compounds of formula I and II can form intramolecular salt bridges instead of associating with counterions represented by M and Y. It is to be understood that in compounds of Formula I and II in which R₁ is —C(R₁₀)(R₁₁)—OPO₃MY or —CH(R₁₀)(R₁₁)—OP(O)₂(OR₂₁)M, it is possible for the phosphate moiety to serve as X— and for the quaternary ammonium group to serve as M.

Substituents indicated as attached through variable points of attachments can be attached to any available position on the ring structure.

In another embodiment, compounds of the present invention are represented by formulas III, IV, V, VI, VII, VIII, IX, and X as illustrated below, or the geometric isomers, enantiomers, diastereomers, racemates, pharmaceutically acceptable salts, co-crystals or solvates thereof:

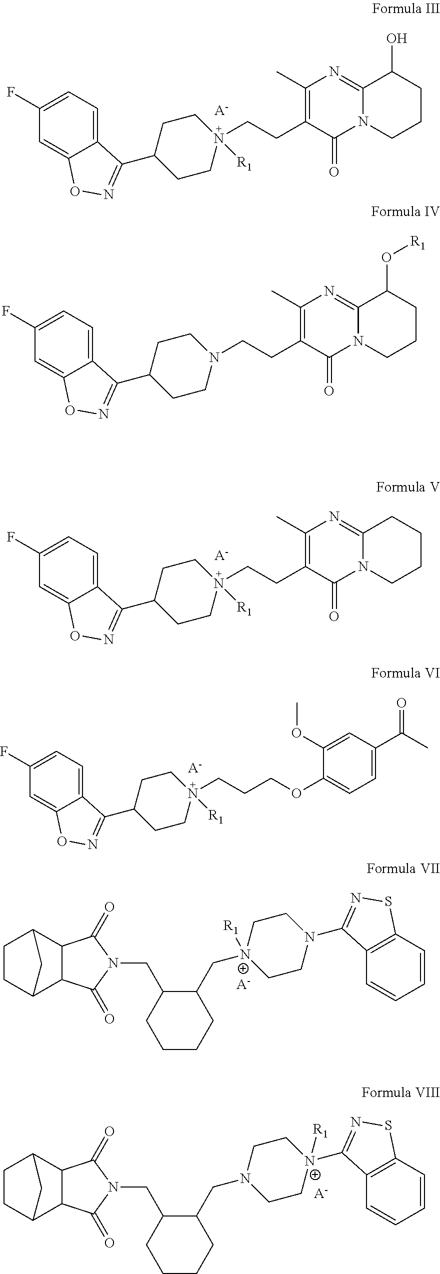

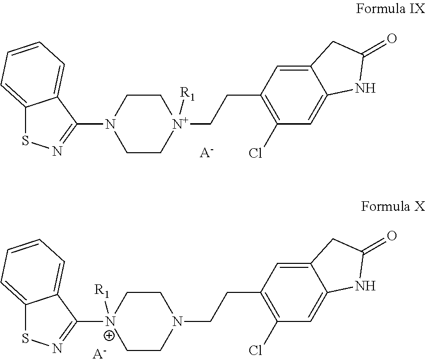

wherein R₁ and A− are as defined above.

In some embodiments, the G₃ moiety is selected from:

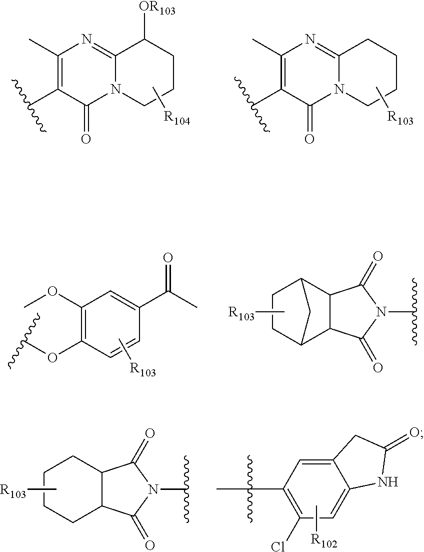

wherein each R₁₀₂, R₁₀₃ and R₁₀₄ are independently selected from hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ alkylamino and optionally substituted C₁-C₈ aryl.

In some embodiments, the R₁ moiety is selected from:

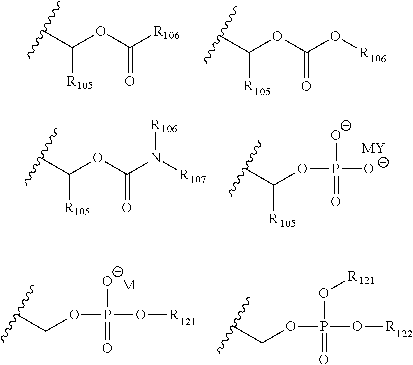

wherein R₁₀₅, R₁₀₆ and R₁₀₇ are independently selected from hydrogen, halogen, optionally substituted C₁-C₂₄ alkyl, optionally substituted C₂-C₂₄ alkenyl, optionally substituted C₂-C₂₄ alkynyl, optionally substituted C₃-C₂₄ cycloalkyl, optionally substituted C₁-C₂₄ alkoxy, optionally substituted C₁-C₂₄ alkylamino and optionally substituted C₁-C₂₄ aryl; and
each R₁₂₁ and R₁₂₂ is independently hydrogen, aliphatic, substituted aliphatic, aryl or substituted aryl.
In some embodiments, R₁ is selected from:

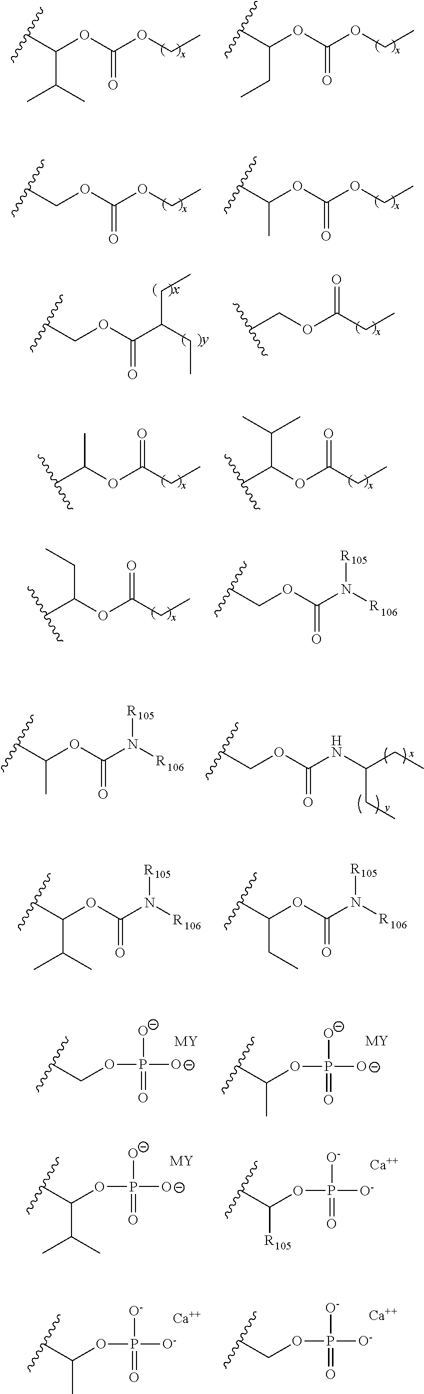

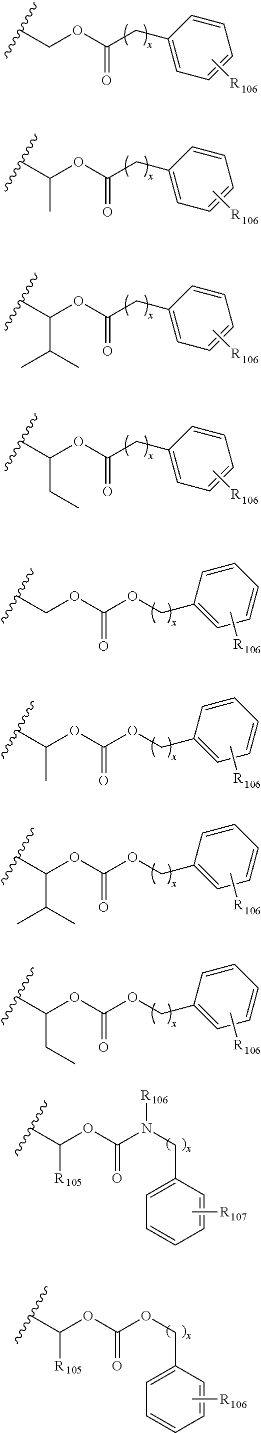

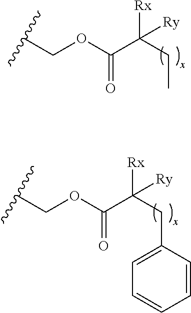

wherein each x and y is independently an integer between 0 and 30;
each Rx and Ry is independently selected from H, halogen, optionally substituted alkyl, or taken together with the carbon to which they are attached form a C₃-C₈ cycloalkyl; and
M, Y, R₁₀₅, R₁₀₆ and R₁₀₇ are as defined above.
In a more preferred embodiment, x is an integer between 5 and 20.
In certain embodiments, R₁ selected from:

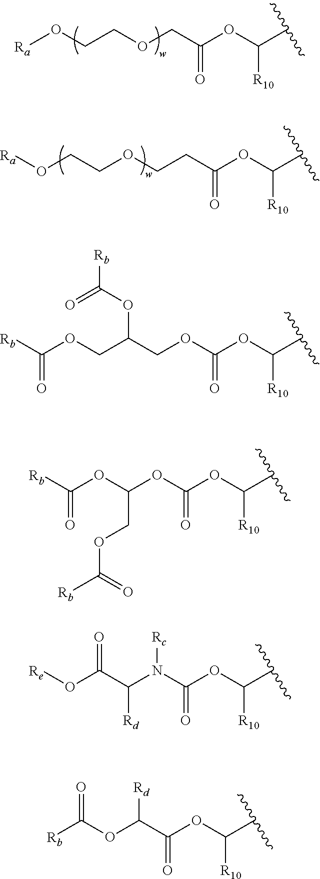

wherein w is 1 to about 1000, preferably 1 to about 100; Rₐ, R_{b} and R_{c} are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl; R_{c} is H or substituted or unsubstituted C₁-C₆-alkyl; R_{d} is H, substituted or unsubstituted C₁-C₆-alkyl, substituted or unsubstituted aryl-C₁-C₆-alkyl or substituted or unsubstituted heteroaryl-C₁-C₆-alkyl; and R₁₀ is as defined above and is preferably hydrogen. Preferably Rₐ, R_{b} and R_{c} are each C₁-C₂₄-alkyl. Preferably R_{d} is the side chain of one of the twenty naturally occurring amino acids, more preferably a neutral or hydrophobic side chain, such as hydrogen, methyl, isopropyl, isobutyl, benzyl, indolylmethyl, and sec-butyl. R_{c} and R_{d} can also, together with the carbon and nitrogen atoms to which they are attached, form a heterocycloalkyl group, preferably a pyrrolidine group.
A more preferred embodiment is selected from:

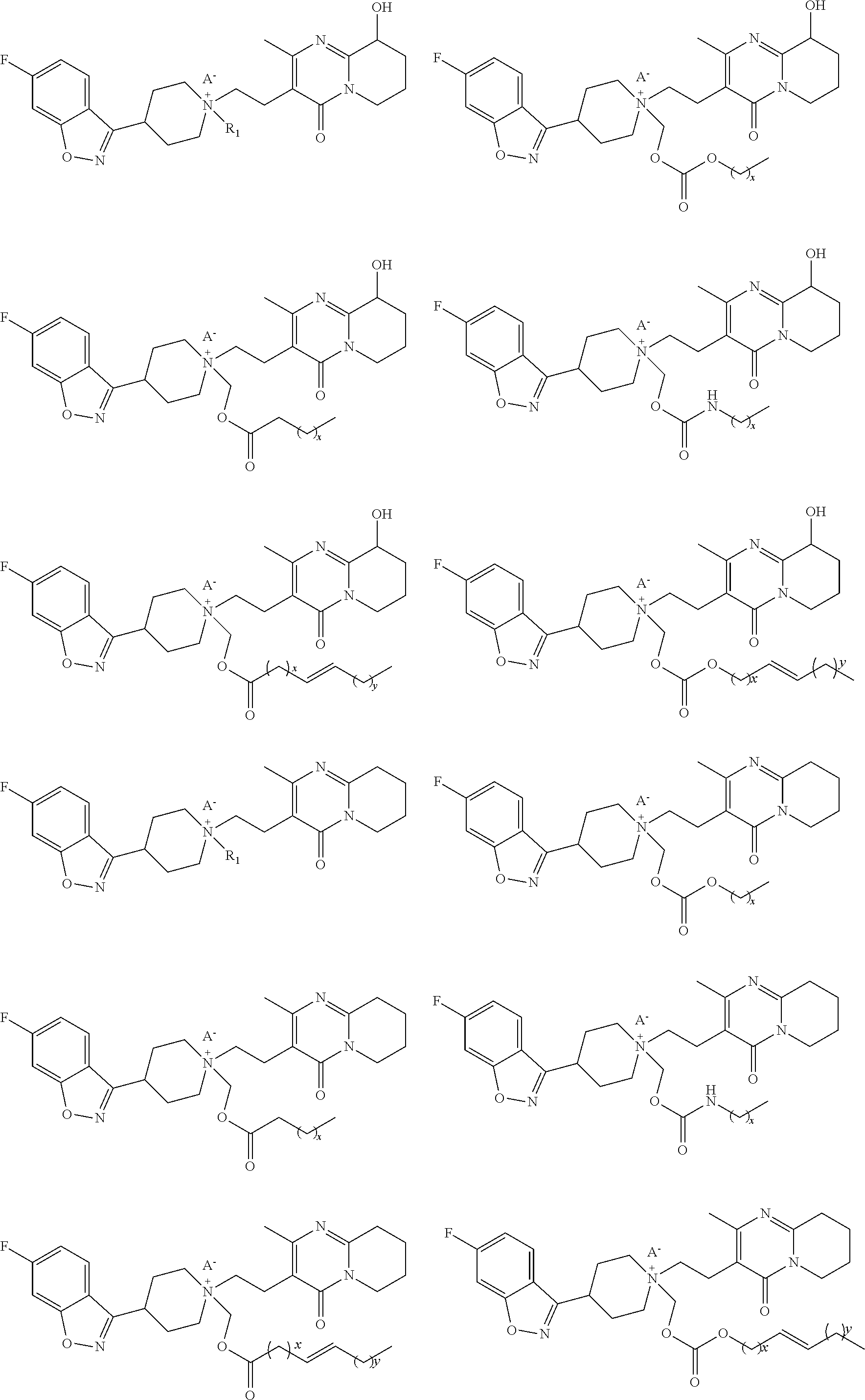

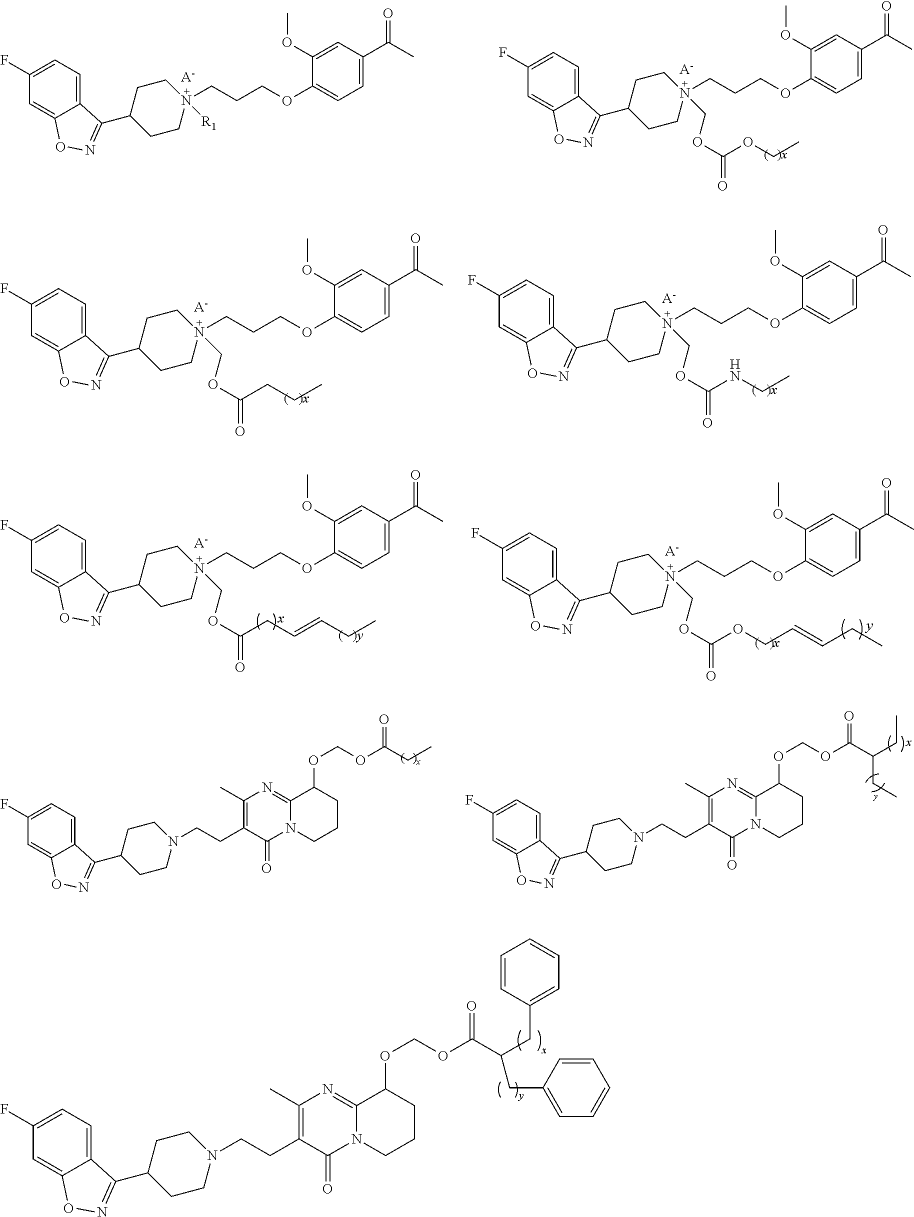

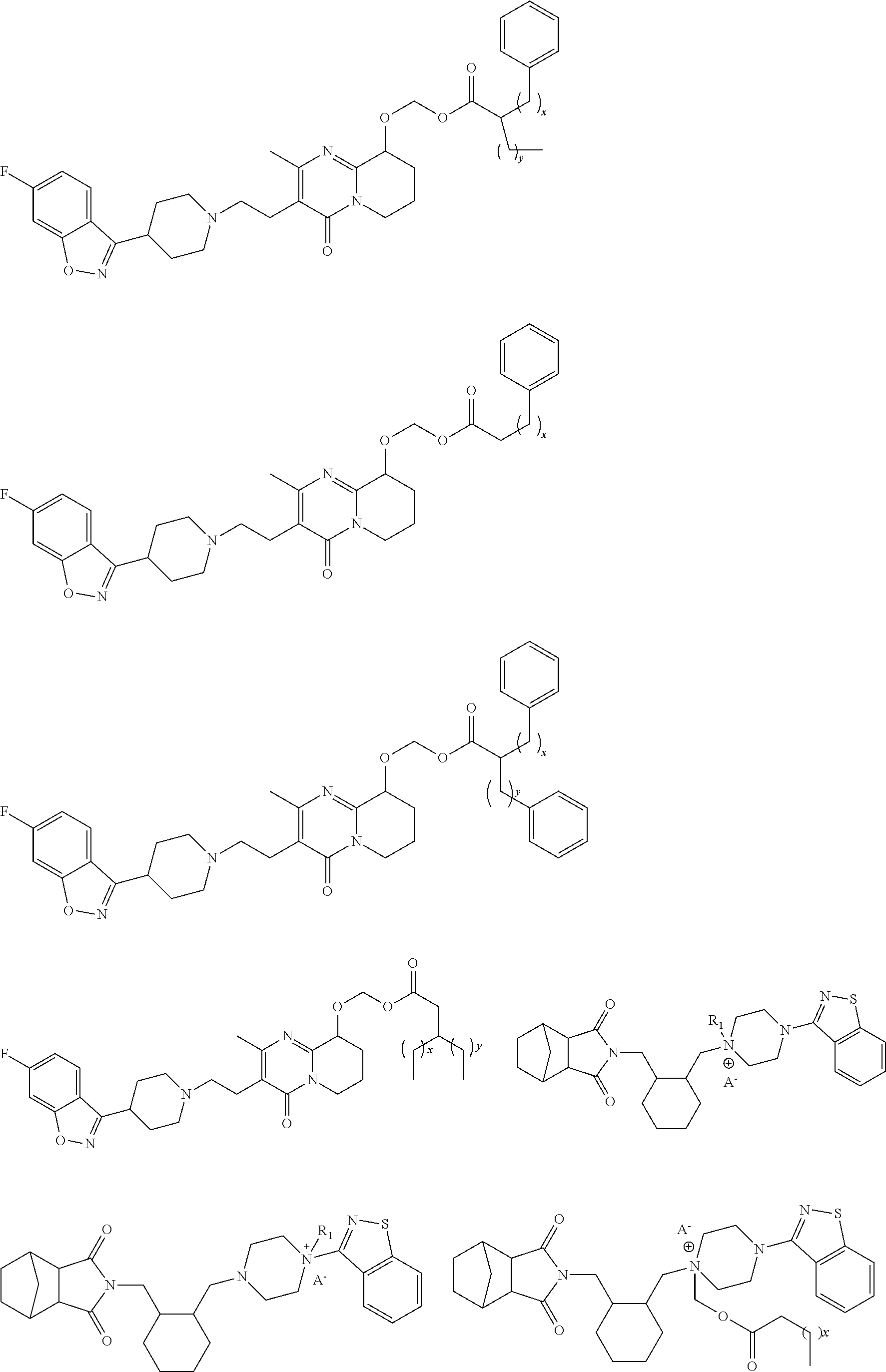

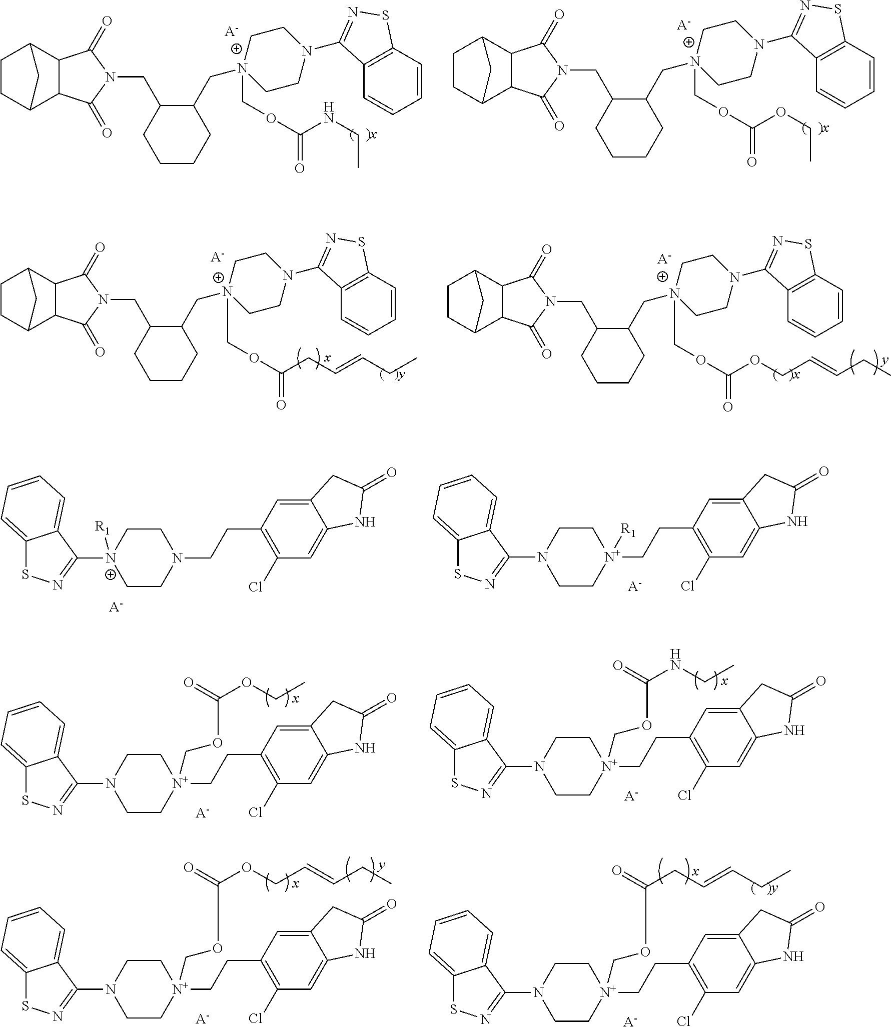

or the geometric isomers, enantiomers, diastereomers, racemates, pharmaceutically acceptable salts or solvates thereof
wherein R₁ and A⁻ are as defined above; and
each x and y is independently an integer between 0 and 30.
In some embodiments, variable R₁ in Formula I is selected from the group set forth in the table below where the variables Y and M as defined above.

TABLE 1

| | |
|---|---|
| 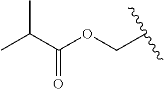 | 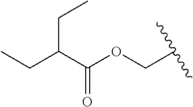 |
| 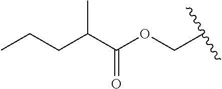 | 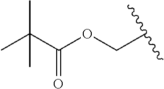 |
| 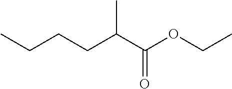 | 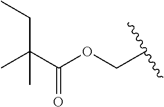 |
| 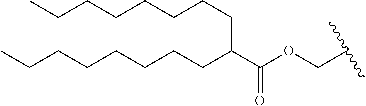 | 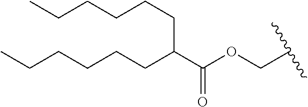 |
| 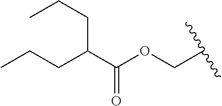 | 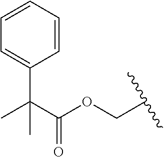 |
| 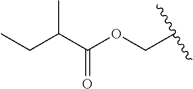 | 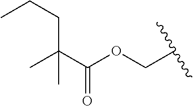 |
| 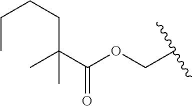 | 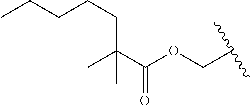 |
| 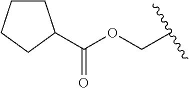 | 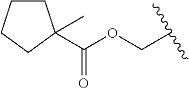 |
| 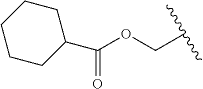 | 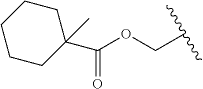 |
| 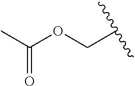 | 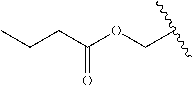 |
| 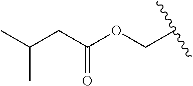 | 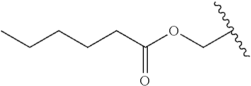 |
| 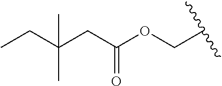 | 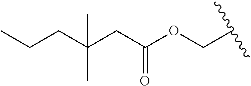 |
| 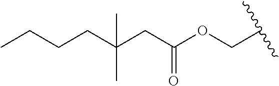 | 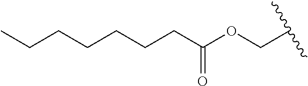 |
| 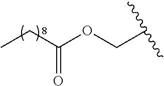 | 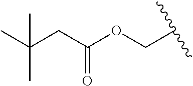 |
| 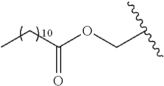 | 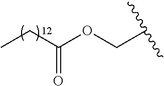 |
| 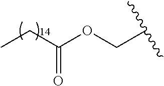 | 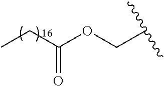 |
| 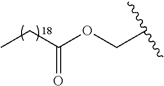 | 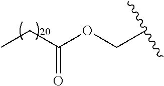 |
| 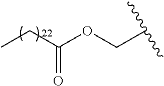 | 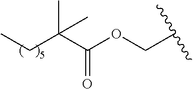 |
| 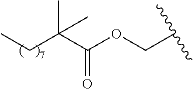 | 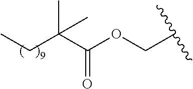 |
| 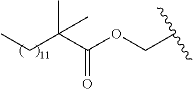 | 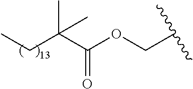 |
| 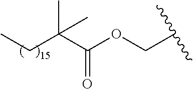 | 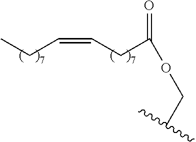 |
| 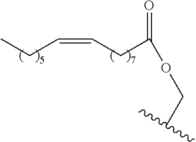 | 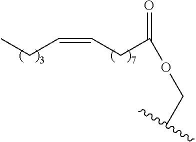 |
| 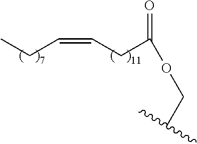 | 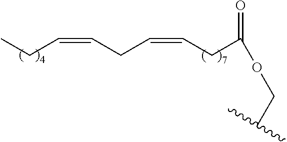 |
| 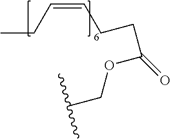 | 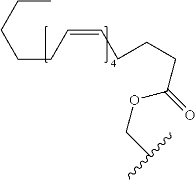 |
| 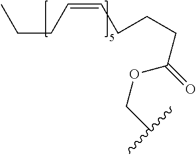 | 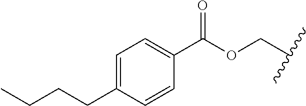 |
| 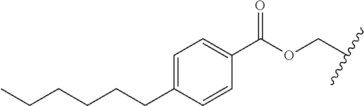 | 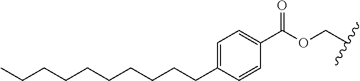 |
| 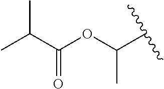 | 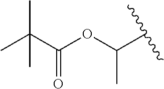 |
| 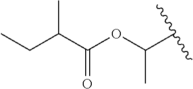 | 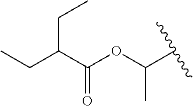 |
| 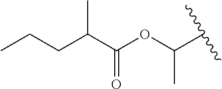 | 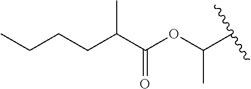 |
| 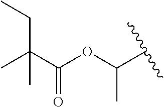 | 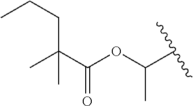 |
| 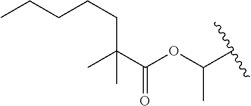 | 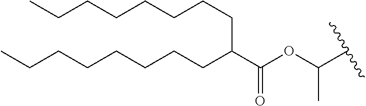 |
| 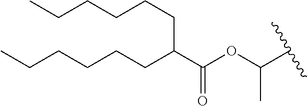 | 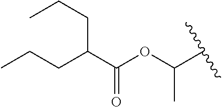 |
| 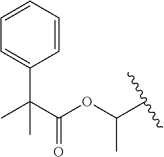 | 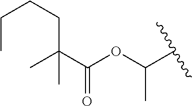 |
| 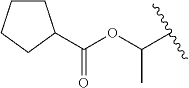 | 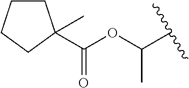 |
| 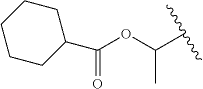 | 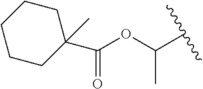 |
| 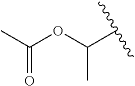 | 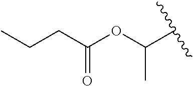 |
| 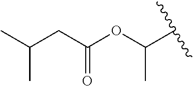 | 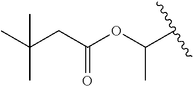 |
| 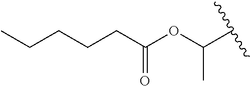 | 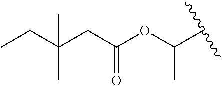 |
| 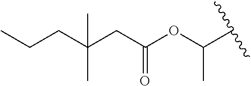 | 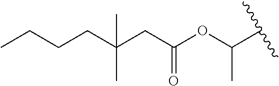 |
| 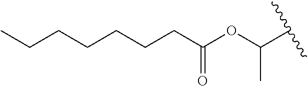 | 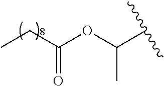 |
| 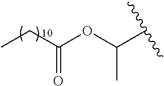 | 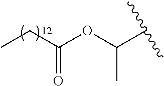 |
| 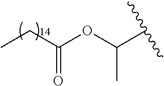 | 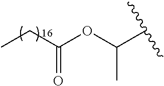 |
| 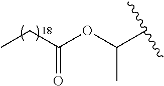 | 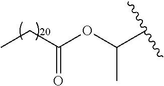 |
| 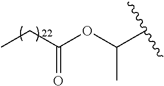 | 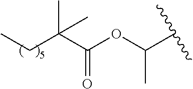 |
| 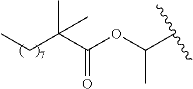 | 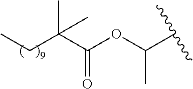 |
| 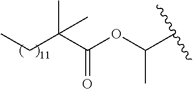 | 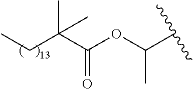 |
| 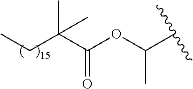 | 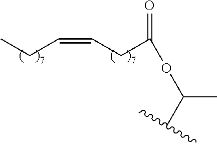 |
| 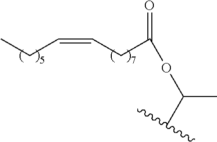 | 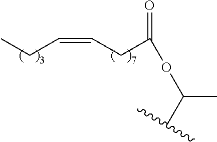 |
| 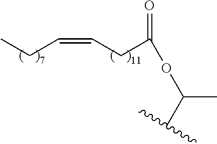 | 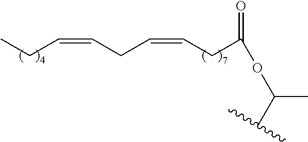 |
| 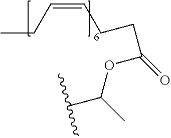 | 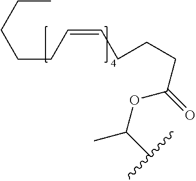 |
| 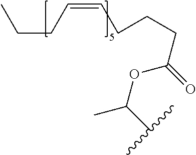 | 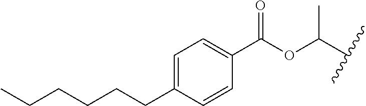 |
| 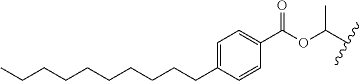 | 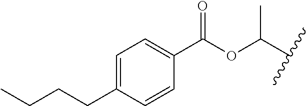 |
| 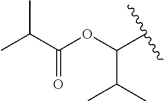 | 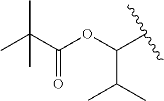 |
| 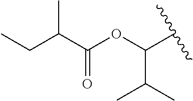 | 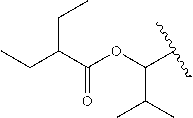 |
| 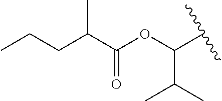 | 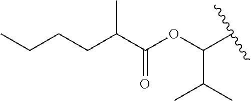 |
| 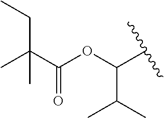 | 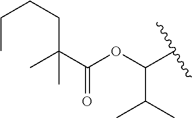 |
| 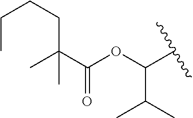 | 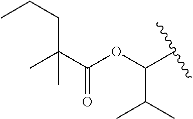 |
| 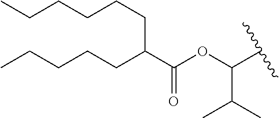 | 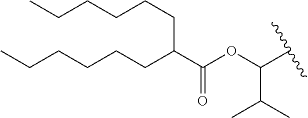 |
| 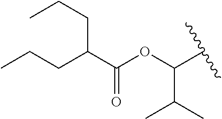 | 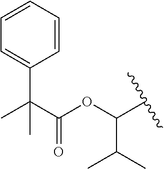 |
| 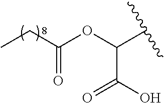 | 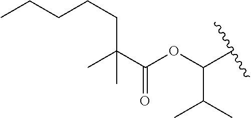 |
| 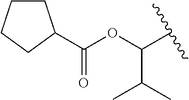 | 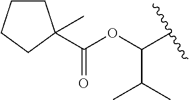 |
| 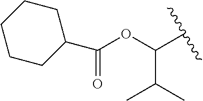 | 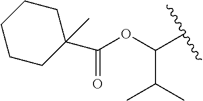 |
| 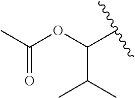 | 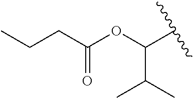 |
| 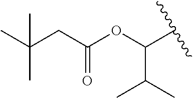 | 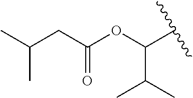 |
| 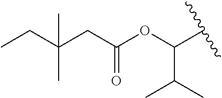 | 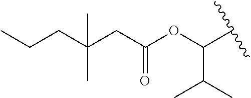 |
| 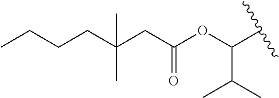 | 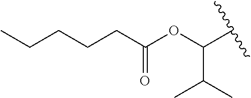 |
| 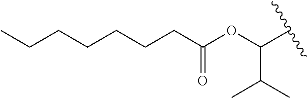 | 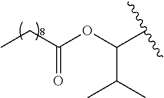 |
| 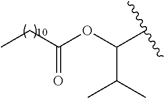 | 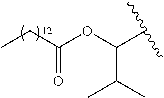 |
| 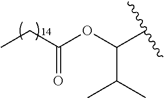 | 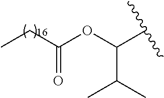 |
| 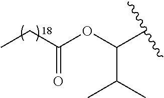 | 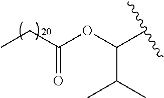 |
| 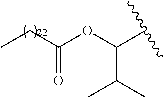 | 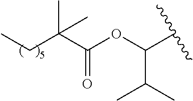 |
| 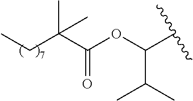 | 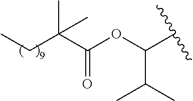 |
| 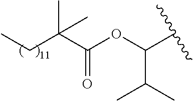 | 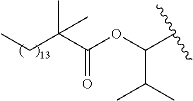 |
| 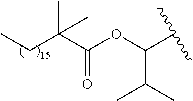 | 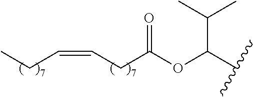 |
| 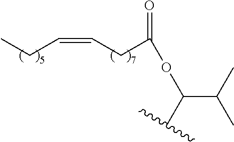 | 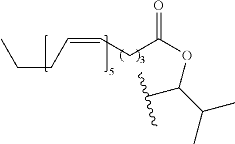 |
| 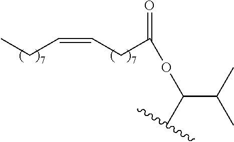 | 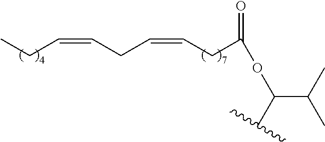 |
| 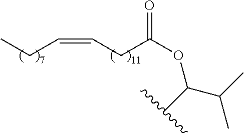 | 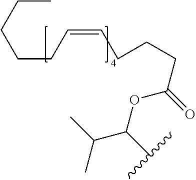 |
| 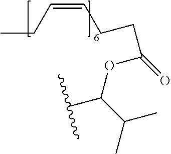 | 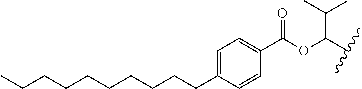 |
| 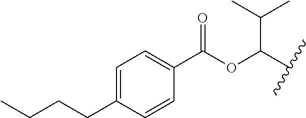 | 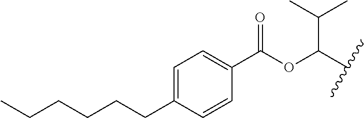 |
| 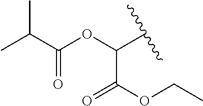 | 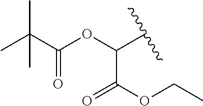 |
| 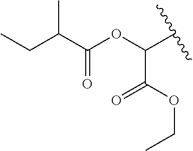 | 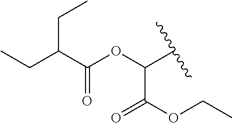 |
| 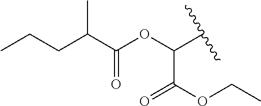 | 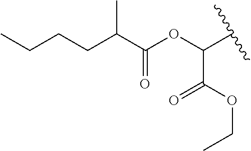 |
| 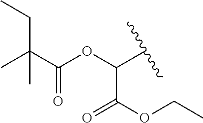 | 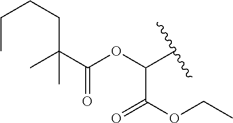 |
| 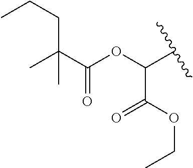 | 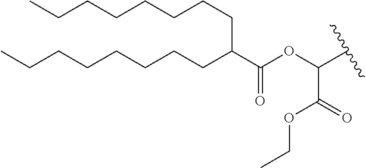 |
| 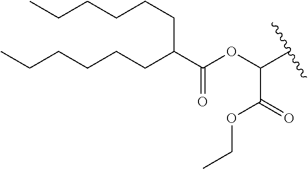 | 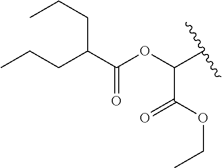 |
| 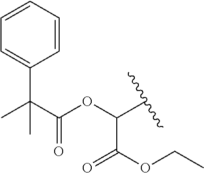 | 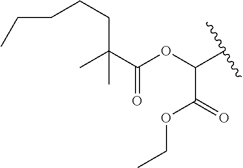 |
| 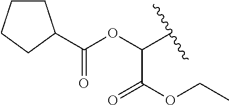 | 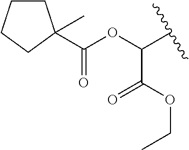 |
| 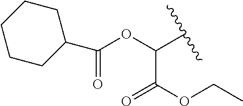 | 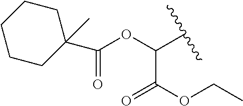 |
| 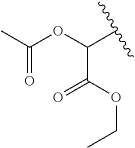 | 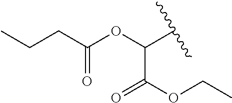 |
| 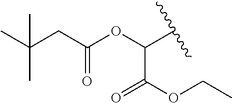 | 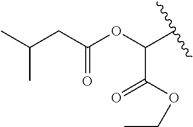 |
| 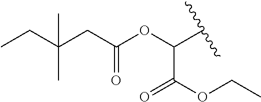 | 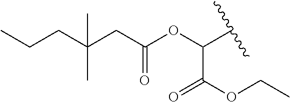 |
| 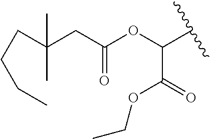 | 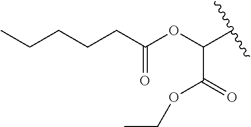 |
| 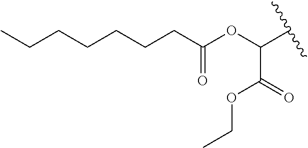 | 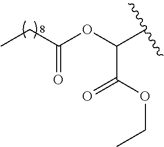 |
| 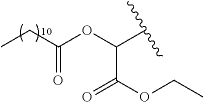 | 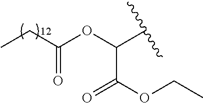 |
| 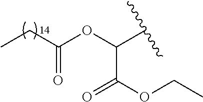 | 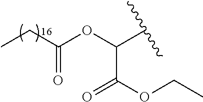 |
| 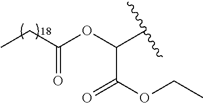 | 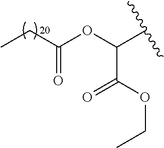 |
| 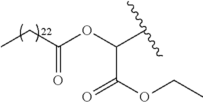 | 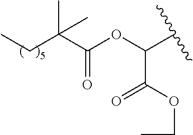 |
| 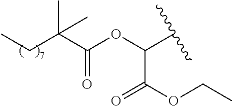 | 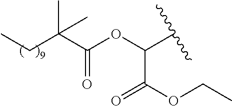 |
| 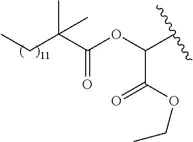 | 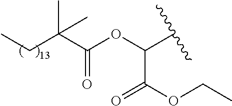 |
| 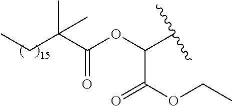 | 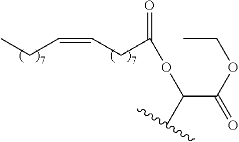 |
| 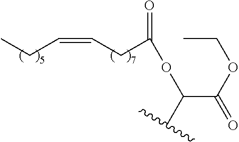 | 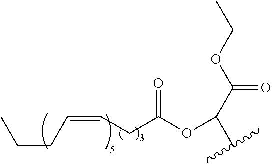 |
| 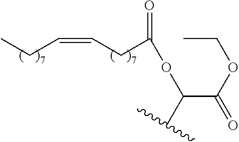 | 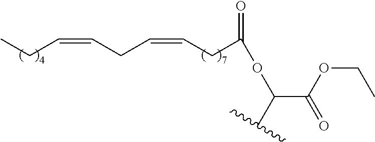 |
| 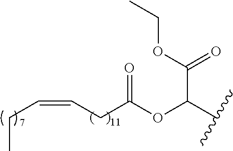 | 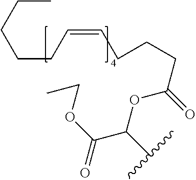 |
| 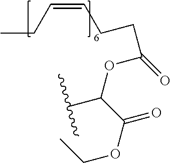 | 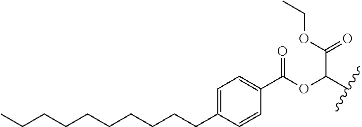 |
| 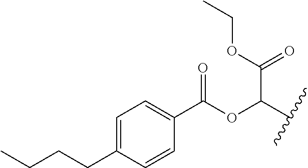 | 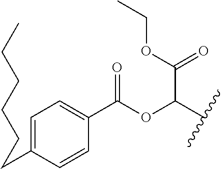 |
| 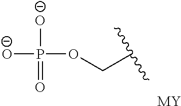 | 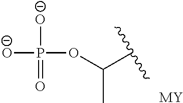 |
| 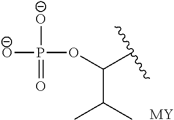 | 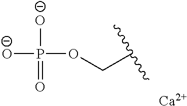 |
| 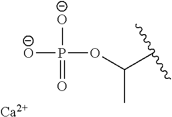 | 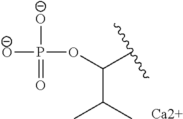 |
| 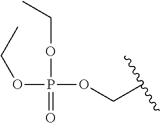 | 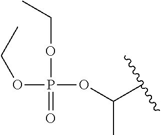 |
| 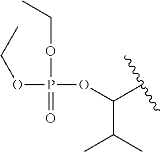 | 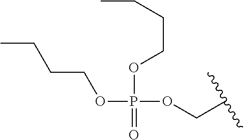 |
| 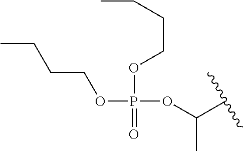 | 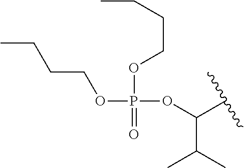 |
| 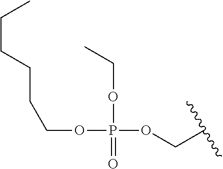 | 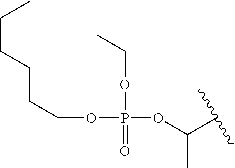 |
| 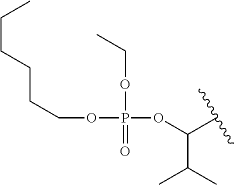 | 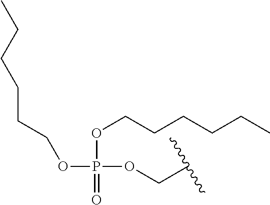 |
| 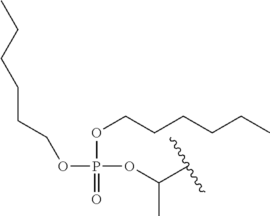 | 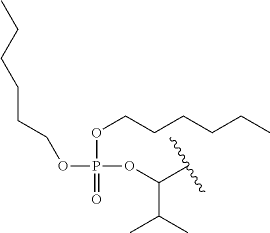 |
| 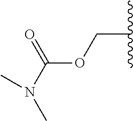 | 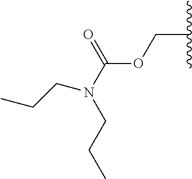 |
| 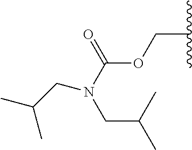 | 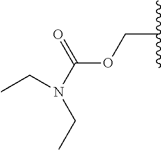 |
| 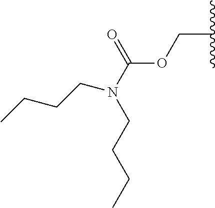 | 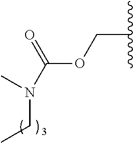 |
| 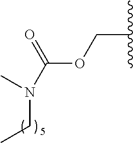 | 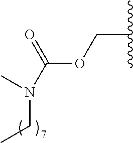 |
| 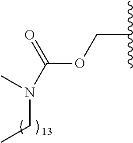 | 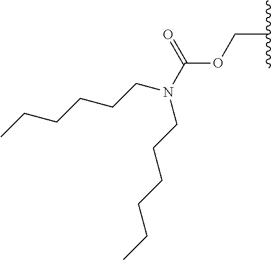 |
| 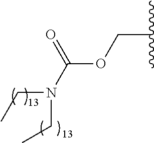 | 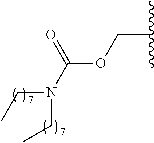 |
| 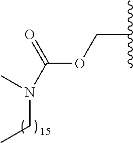 | 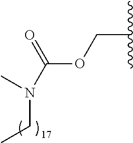 |
| 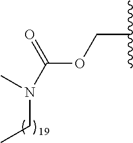 | 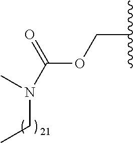 |
| 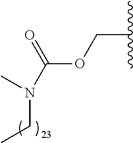 | 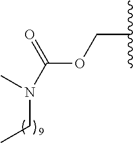 |
| 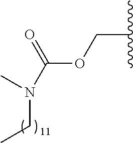 | 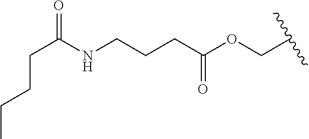 |
| 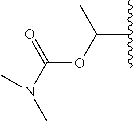 | 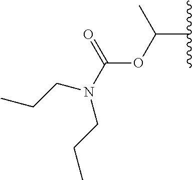 |
| 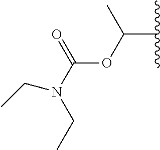 | 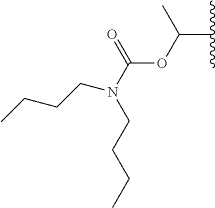 |
| 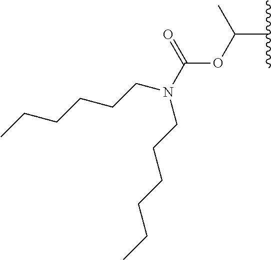 | 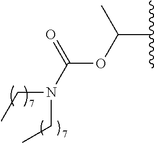 |
| 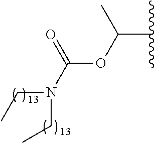 | 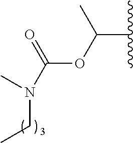 |
| 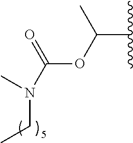 | 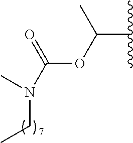 |
| 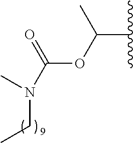 | 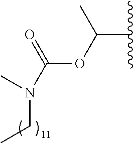 |
| 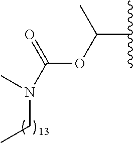 | 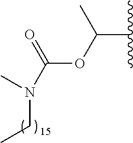 |
| 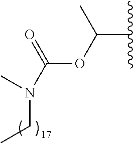 | 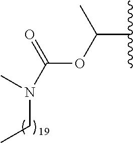 |
| 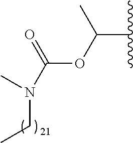 | 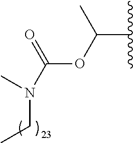 |
| 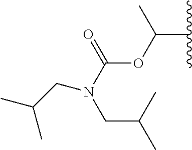 | 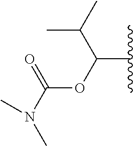 |
| 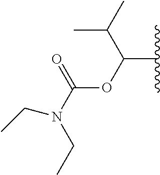 | 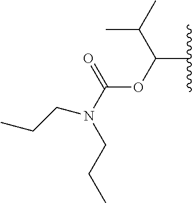 |
| 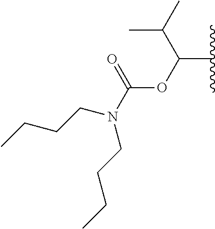 | 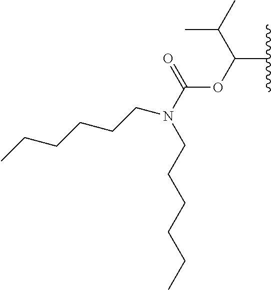 |
| 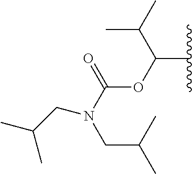 | 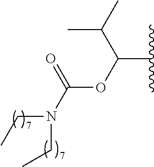 |
| 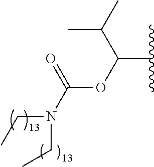 | 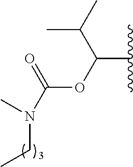 |
| 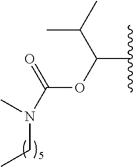 | 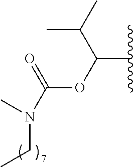 |
| 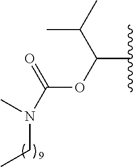 | 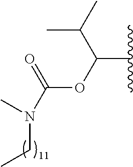 |
| 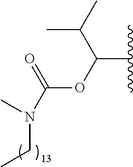 | 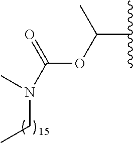 |
| 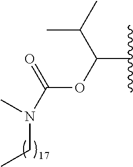 | 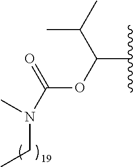 |
| 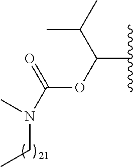 | 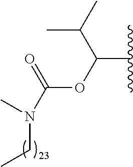 |
| 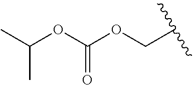 | 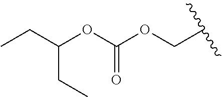 |
| 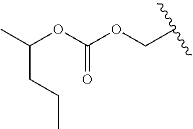 | 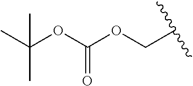 |
| 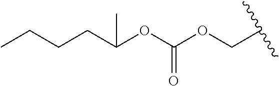 | 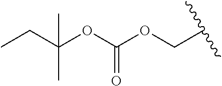 |
| 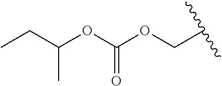 | 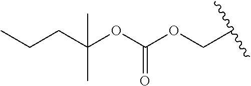 |
| 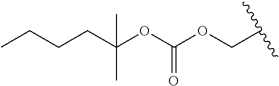 | 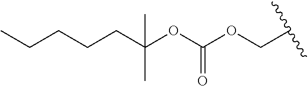 |
| 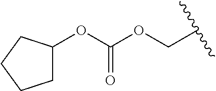 | 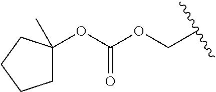 |
| 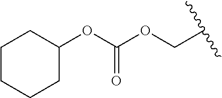 | 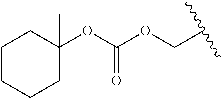 |
| 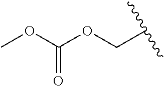 | 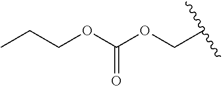 |
| 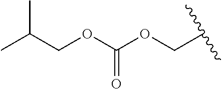 | 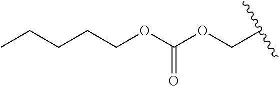 |
| 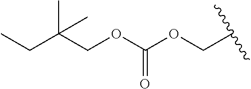 | 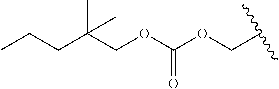 |
| 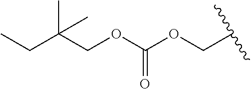 | 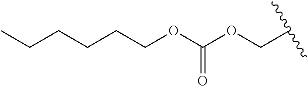 |
| 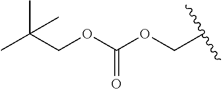 | 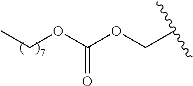 |
| 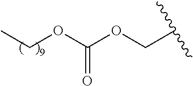 | 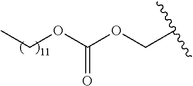 |
| 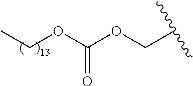 | 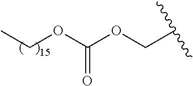 |
| 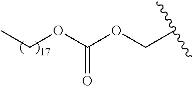 | 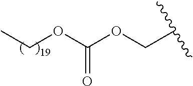 |
| 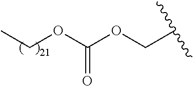 | 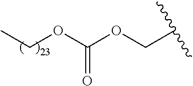 |
| 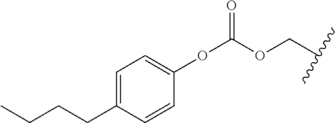 | 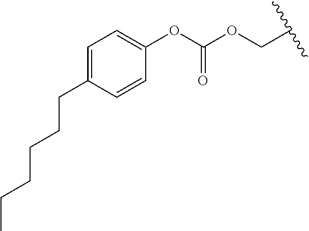 |
| 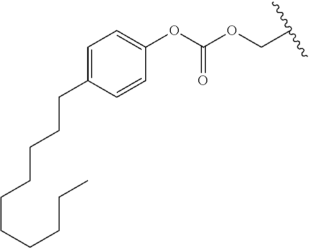 | 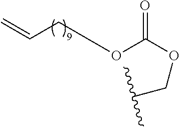 |
| 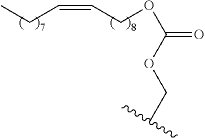 | 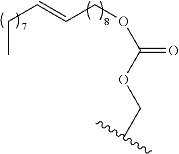 |
| 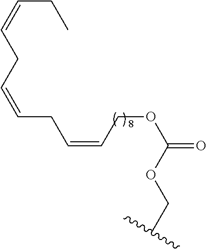 | 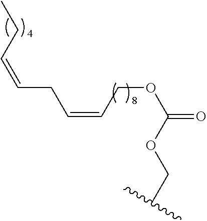 |
| 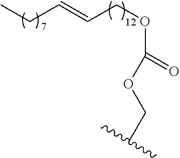 | 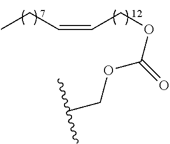 |
| 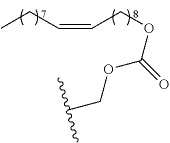 | 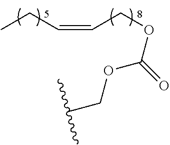 |
| 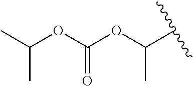 | 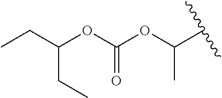 |
| 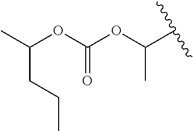 | 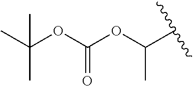 |
| 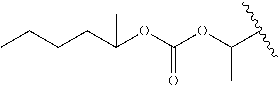 | 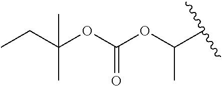 |
| 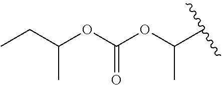 | 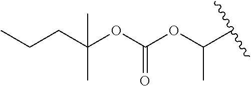 |
| 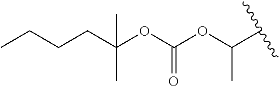 | 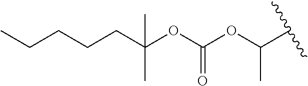 |
| 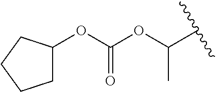 | 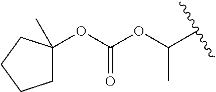 |
| 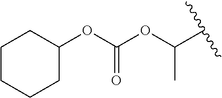 | 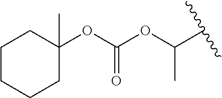 |
| 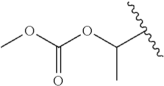 | 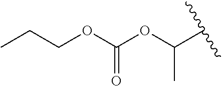 |
| 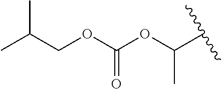 | 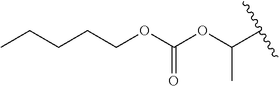 |
| 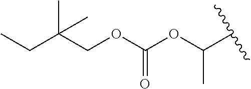 | 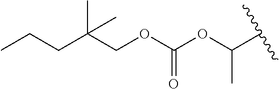 |
| 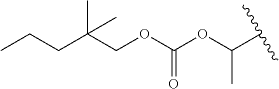 | 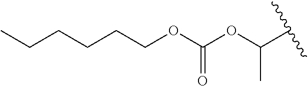 |
| 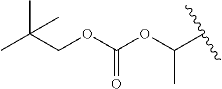 | 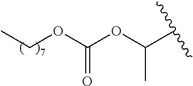 |
| 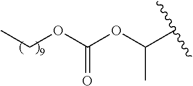 | 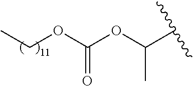 |
| 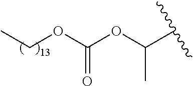 | 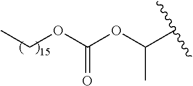 |
| 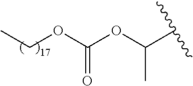 | 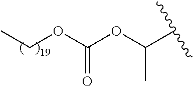 |
| 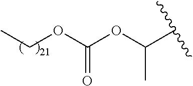 | 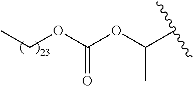 |
| 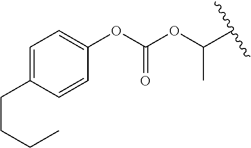 | 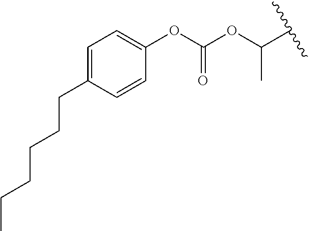 |
| 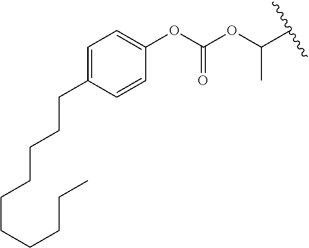 | 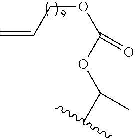 |
| 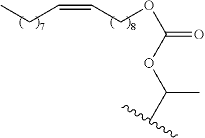 | 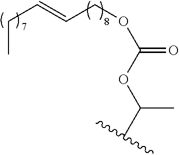 |
| 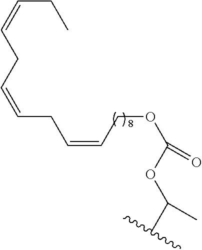 | 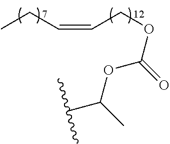 |
| 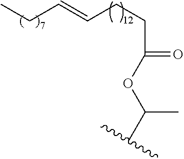 | 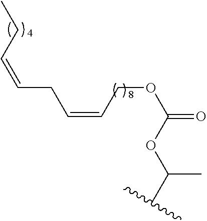 |
| 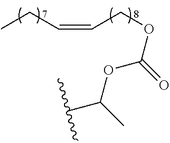 | 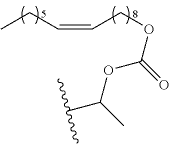 |
| 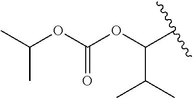 | 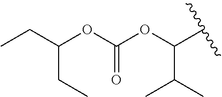 |
| 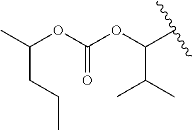 | 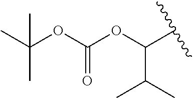 |
| 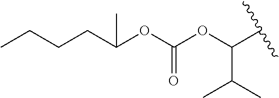 | 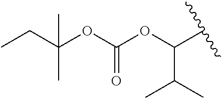 |
| 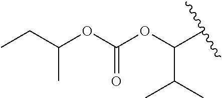 | 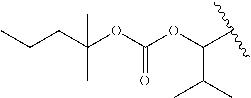 |
| 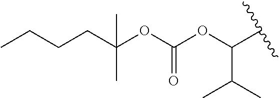 | 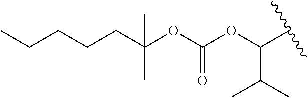 |
| 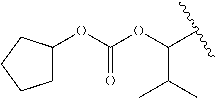 | 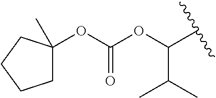 |
| 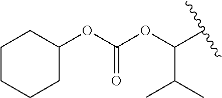 | 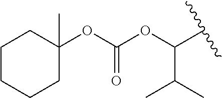 |
| 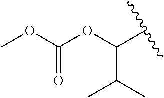 | 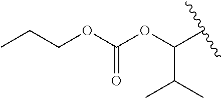 |
| 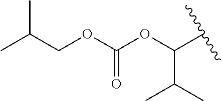 | 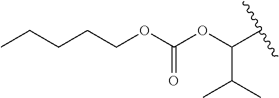 |
| 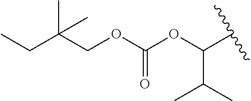 | 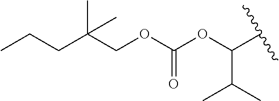 |
| 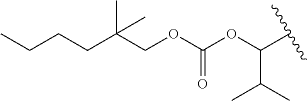 | 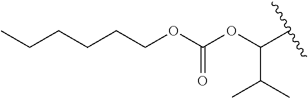 |
| 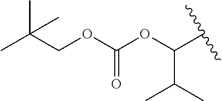 | 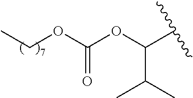 |
| 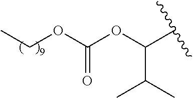 | 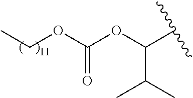 |
| 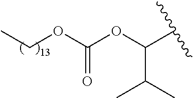 | 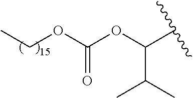 |
| 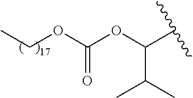 | 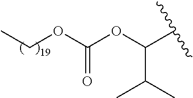 |
| 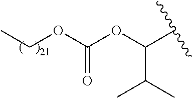 | 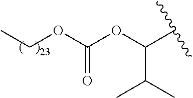 |
| 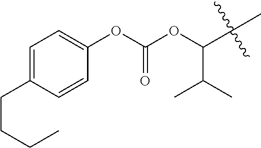 | 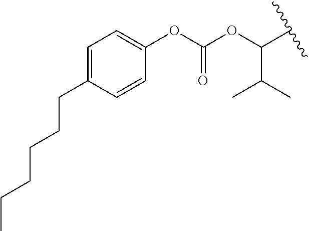 |
| 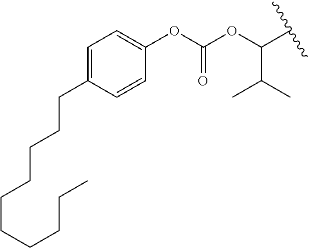 | 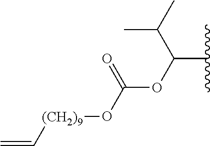 |
| 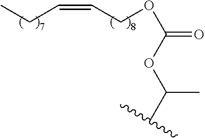 | 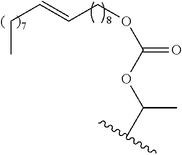 |
| 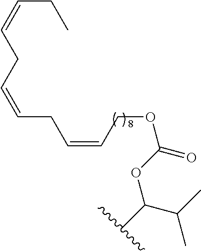 | 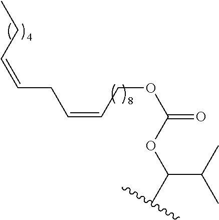 |
| 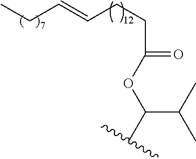 | 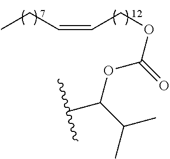 |
| 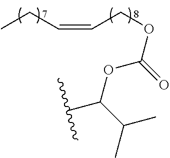 | 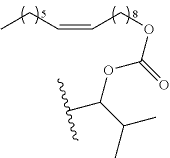 |

In some embodiments, variable R₁ in any of Formulas I through X is selected from the group set forth in Tables 2, 3, 4 and 5 below.

TABLE 2

| | |
|---|---|
| 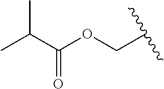 | 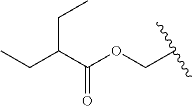 |
| 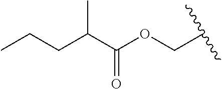 | 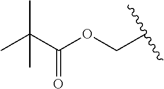 |
| 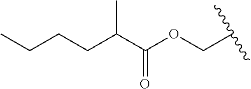 | 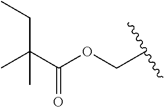 |
| 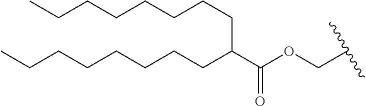 | 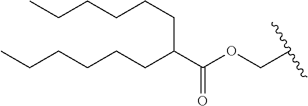 |
| 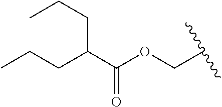 | 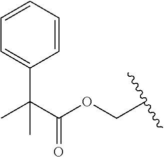 |
| 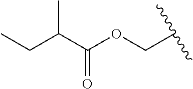 | 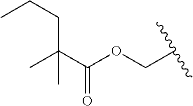 |
| 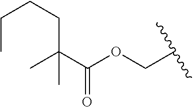 | 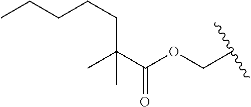 |
| 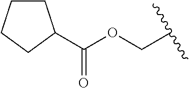 | 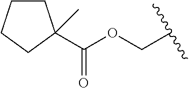 |
| 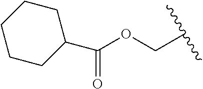 | 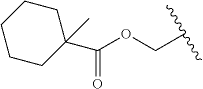 |
| 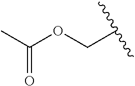 | 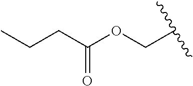 |
| 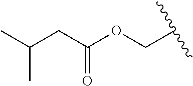 | 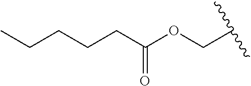 |
| 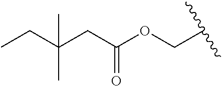 | 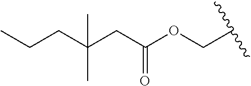 |
| 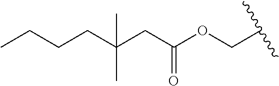 | 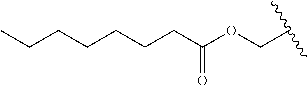 |
| 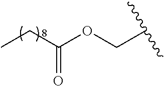 | 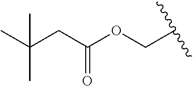 |
| 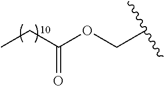 | 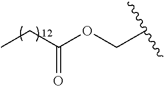 |
| 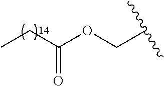 | 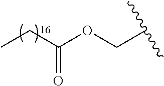 |
| 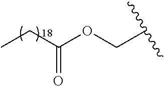 | 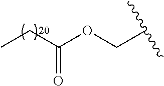 |
| 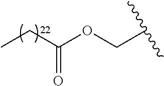 | 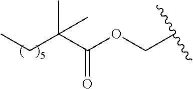 |
| 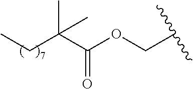 | 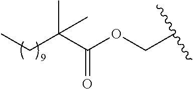 |
| 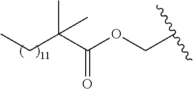 | 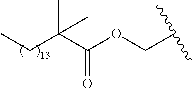 |
| 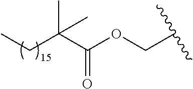 | 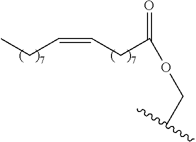 |
| 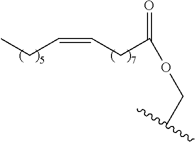 | 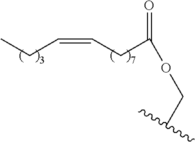 |
| 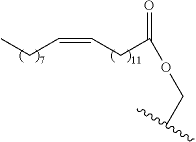 | 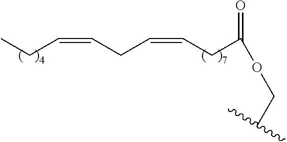 |
| 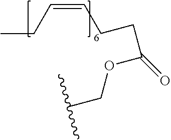 | 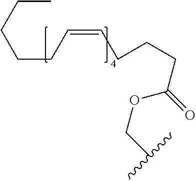 |
| 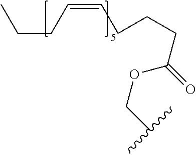 | 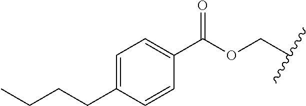 |
| 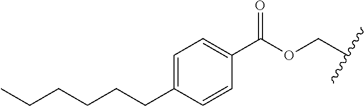 | 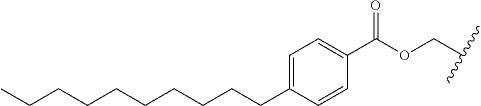 |

TABLE 3

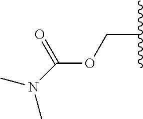

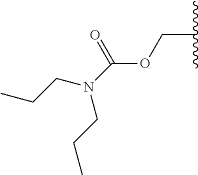

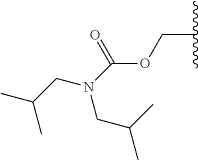

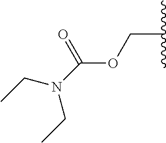

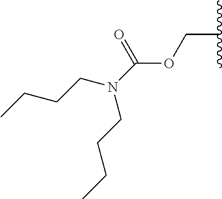

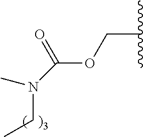

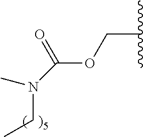

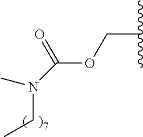

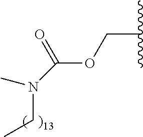

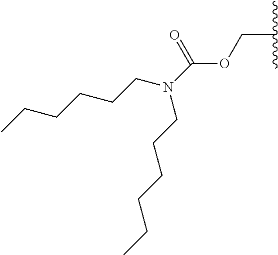

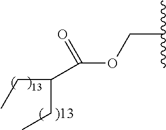

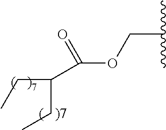

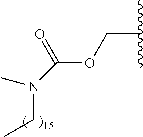

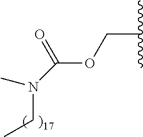

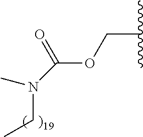

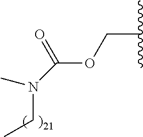

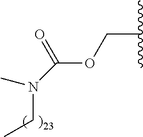

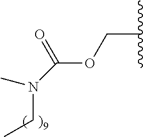

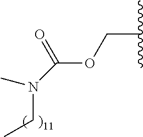

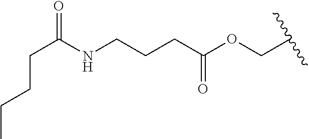

TABLE 4

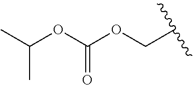

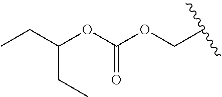

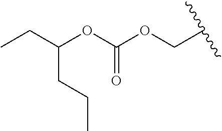

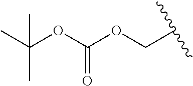

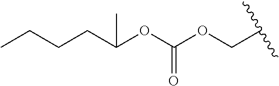

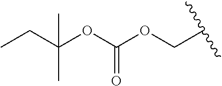

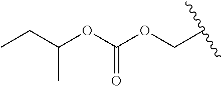

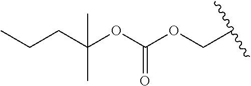

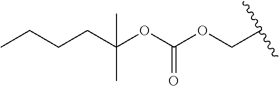

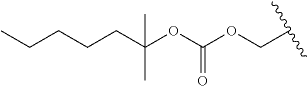

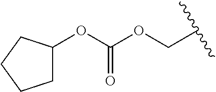

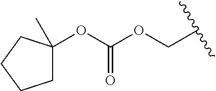

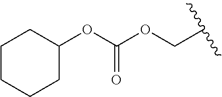

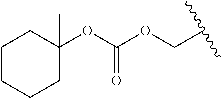

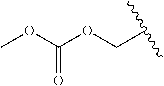

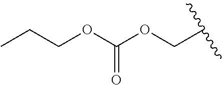

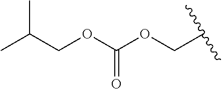

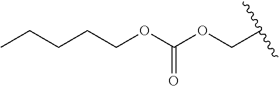

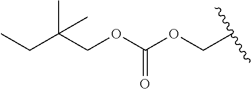

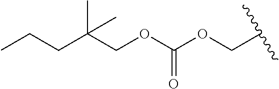

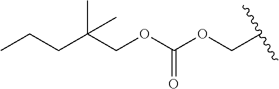

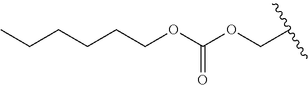

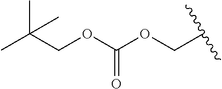

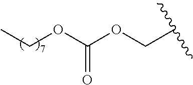

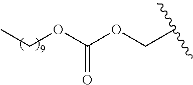

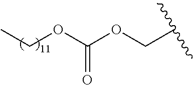

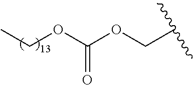

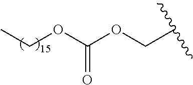

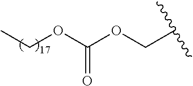

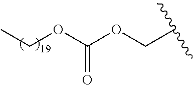

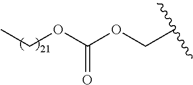

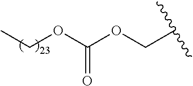

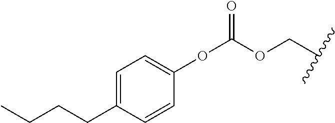

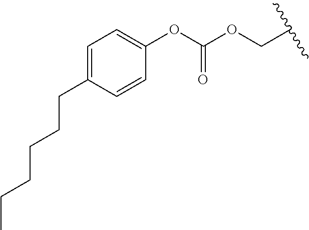

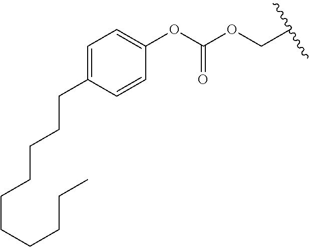

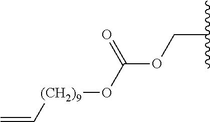

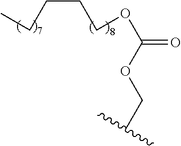

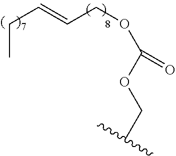

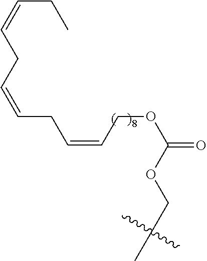

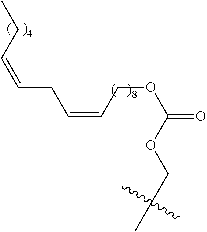

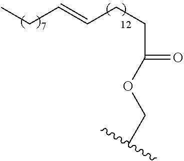

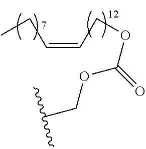

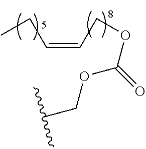

TABLE 5

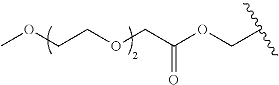

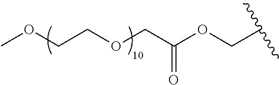

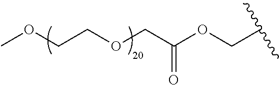

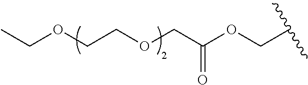

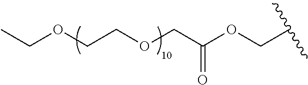

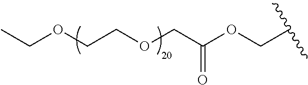

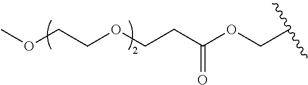

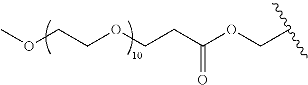

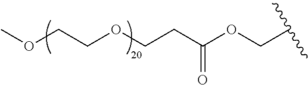

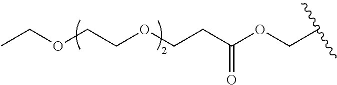

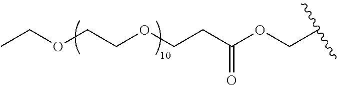

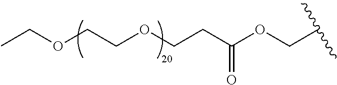

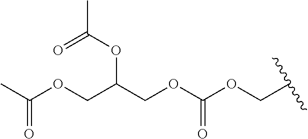

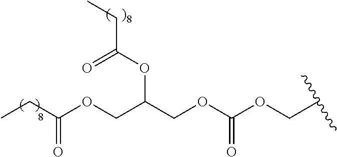

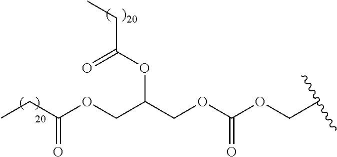

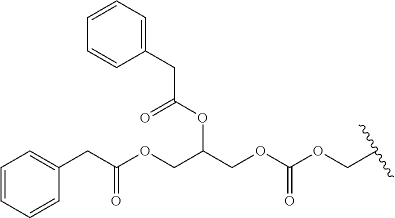

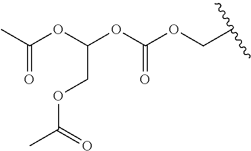

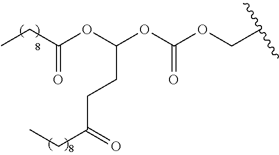

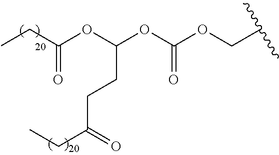

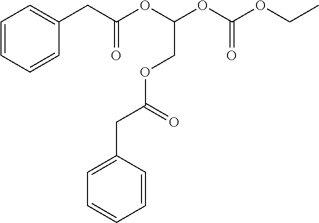

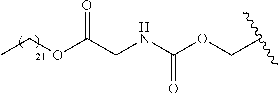

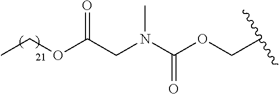

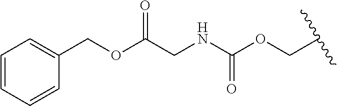

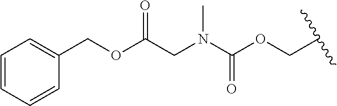

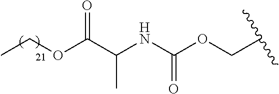

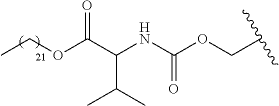

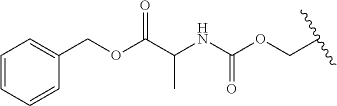

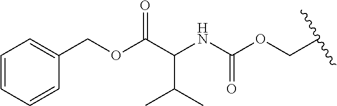

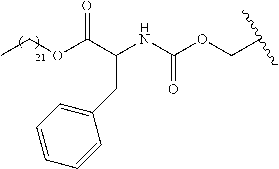

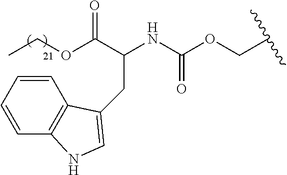

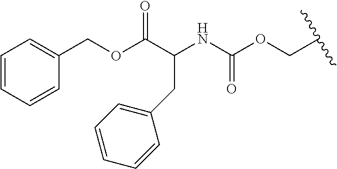

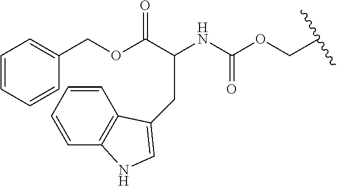

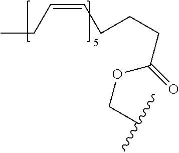

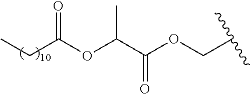

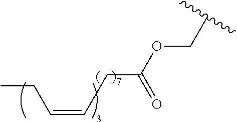

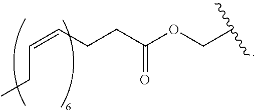

A preferred embodiment is a compound of formula III, wherein R₁ is selected from Table 1, and A⁻ is chloride:

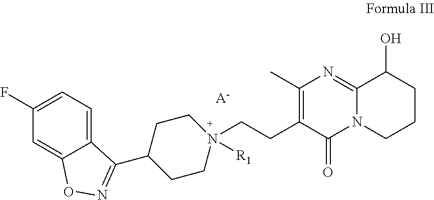

Another preferred embodiment is a compound of formula III, wherein R₁ is selected from Table 1, and A⁻ is bromide or iodide.

A preferred compound is a compound of formula IV, wherein R₁ is selected from Table 1:

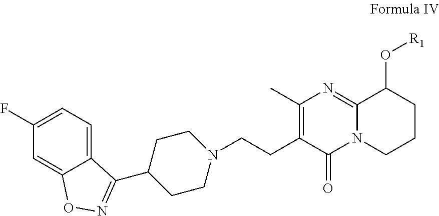

A preferred embodiment is a compound of formula V, wherein R₁ is selected from Table 1, and A⁻ is chloride:

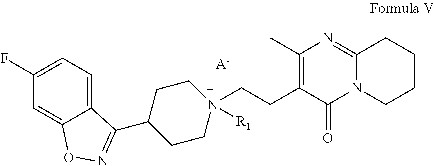

Another preferred embodiment is a compound of formula V, wherein R₁ is selected from Table 1, and A⁻ is bromide or iodide.

A preferred embodiment is a compound of formula VI, wherein R₁ is selected from Table 1, and A⁻ is chloride:

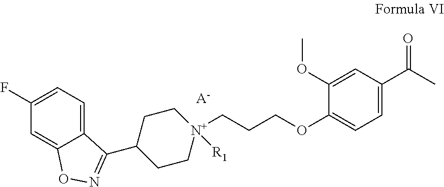

Another preferred embodiment is a compound of formula VI, wherein R₁ is selected from Table 1, and A⁻ is bromide or iodide.

A preferred embodiment is a compound of formula VII, wherein R₁ is selected from Table 1, and A⁻ is chloride:

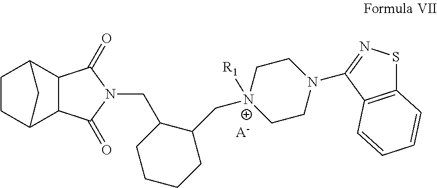

Another preferred embodiment is a compound of formula VII, wherein R₁ is selected from Table 1, and A⁻ is bromide or iodide.

A preferred embodiment is a compound of formula VIII, wherein R₁ is selected from Table 1, and A⁻ is chloride:

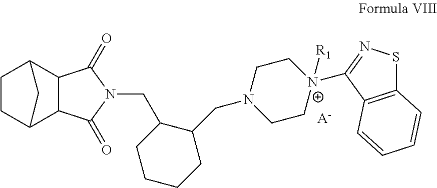

Another preferred embodiment is a compound of formula VIII, wherein R₁ is selected from Table 1, and A⁻ is bromide or iodide.

A preferred embodiment is a compound of formula IX, wherein R₁ is selected from Table 1, and A⁻ is chloride:

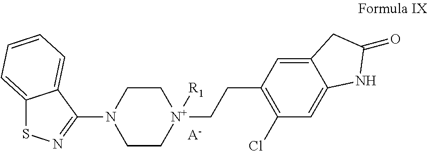

Another preferred embodiment is a compound of formula IX, wherein R₁ is selected from Table 1, and A⁻ is bromide or iodide.

A preferred embodiment is a compound of formula X, wherein R₁ is selected from Table 1, and A⁻ is chloride:

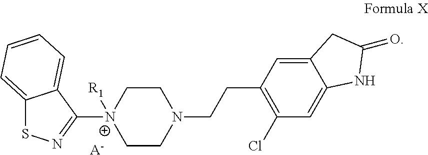

Another preferred embodiment is a compound of formula X, wherein R₁ is selected from Table 1, and A⁻ is bromide or iodide.

A preferred embodiment is a compound of formula X, wherein R₁ is selected from Table 1, and A⁻ is chloride.

The compounds of the invention can be prepared as acid addition salts. Preferably, the acid is a pharmaceutically acceptable acid. Such acids are described in Stahl, P. H. and Wermuth, C. G. (eds.), Handbook of Pharmaceutical Salts: Properties, Selection and Use, Wiley VCH (2008). Pharmaceutically acceptable acids include acetic acid, dichloroacetic acid, adipic acid, alginic acid, L-ascorbic acid, L-aspartic acid, benzenesulfonic acid, 4-acetamidobenzoic acid, benzoic acid, p-bromophenylsulfonic acid; (+)-camphoric acid, (+)-camphor-10-sulfonic acid, capric acid, caproic acid, caprylic acid, carbonic acid, cinnamic acid, cyclamic acid, dodecylsulfuric acid, ethane-1,2-disulfonic acid, ethanesulfonic acid, 2-hydroxyethanesulfonic acid, sulfuric acid, boric acid, citric acid, formic acid, fumaric acid, galactaric acid, gentisic acid, D-glucoheptonic acid, D-gluconic acid, D-glucuronic acid, glutamic acid, glutaric acid, 2-oxoglutaric acid, glycerophosphoric acid, glycolic acid, hippuric acid, hydrochloric acid, hydrobromic acid, hydroiodic acid, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, oleic acid, orotic acid, oxalic acid, palmitic acid, pamoic acid, phosphoric acid, propionic acid, (−)-L-pyroglutamic acid, salicyclic acid, 4-aminosalicyclic acid, sebacic acid, stearic acid, succinic acid, (+)-L-tartaric acid, thiocyanic acid, p-toluenesulfonic acid, and undecylenic acid.

The term “pharmaceutically acceptable anion” as used herein, refers to the conjugate base of a pharmaceutically acceptable acid. Such anions include the conjugate base of any the acids set forth above. Preferred pharmaceutically acceptable anions include acetate, bromide, camsylate, chloride, formate, fumarate, iodide, malate, maleate, mesylate, nitrate, oxalate, phosphate, sulfate, tartrate, thiocyanate and tosylate.

Representative compounds according to the invention are those selected from the Table A below or the geometric isomers, enantiomers, diastereomers, racemates, pharmaceutically acceptable salts, prodrugs or solvates thereof. These are all represented as chloride or iodide salts; however the compounds can be prepared as salts of other pharmaceutically acceptable anions. Selection of a suitable anion can be made on a case-by-case basis to modulate the solubility and/or delivery properties of the material. Anions may be generalized to A⁻.

TABLE A

| | |
|---|---|
| 1 | 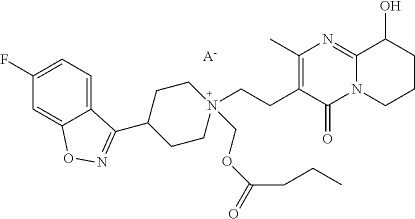 |
| 2 | 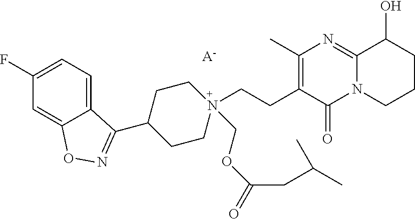 |
| 3 | 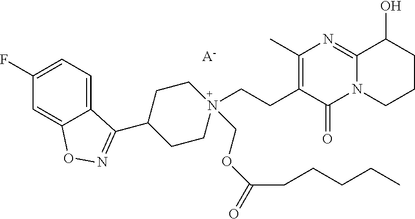 |
| 4 | 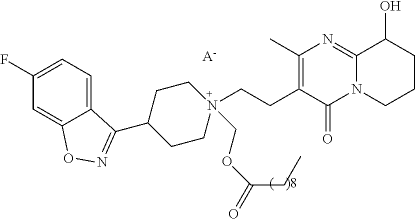 |
| 5 | 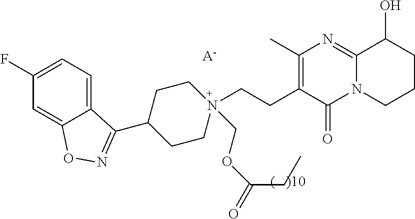 |
| 6 | 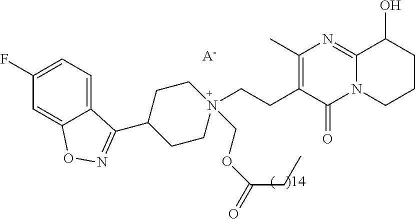 |
| 7 | 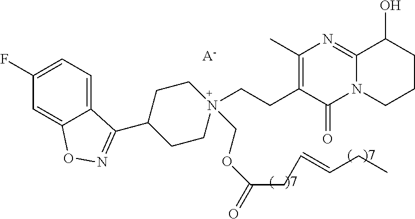 |
| 8 | 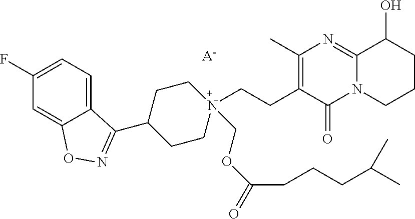 |
| 9 | 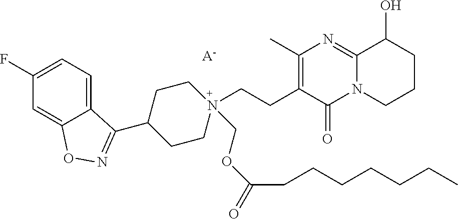 |
| 10 | 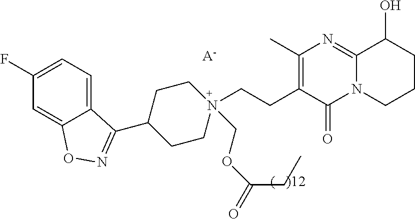 |
| 11 | 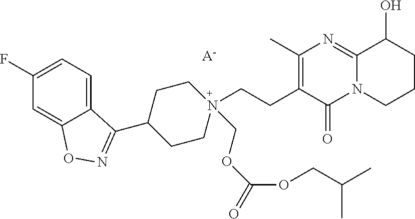 |
| 12 | 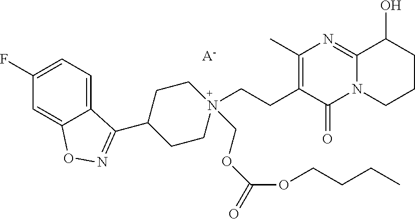 |
| 13 | 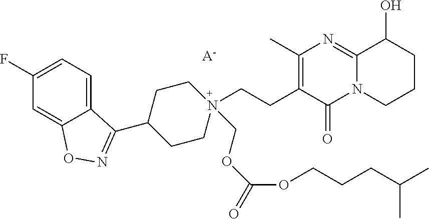 |
| 14 | 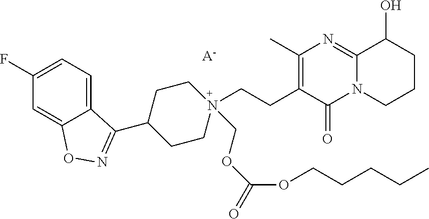 |
| 15 | 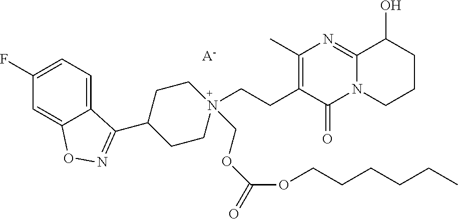 |
| 16 | 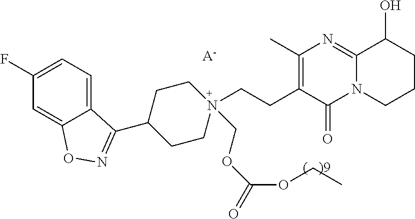 |
| 17 | 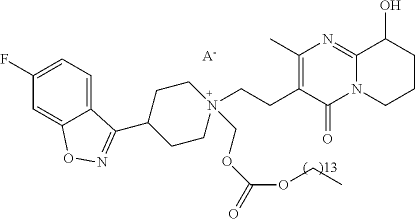 |
| 18 | 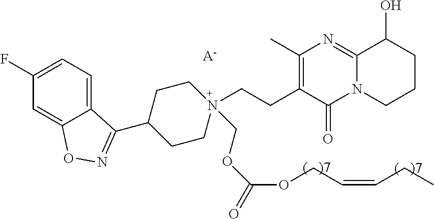 |
| 19 | 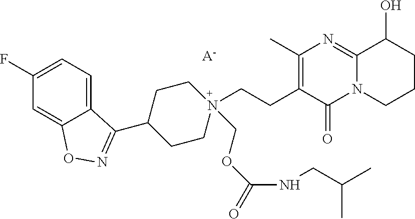 |
| 20 | 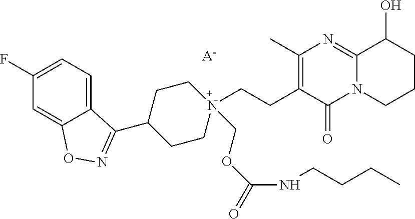 |
| 21 | 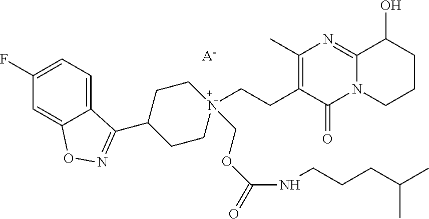 |
| 22 | 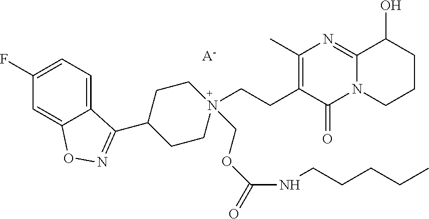 |
| 23 | 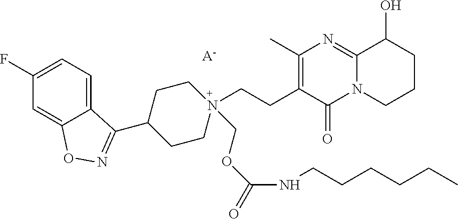 |
| 24 | 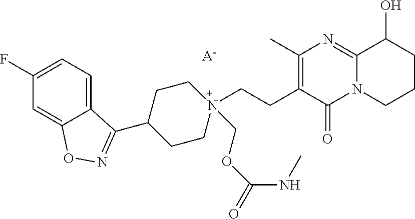 |
| 25 | 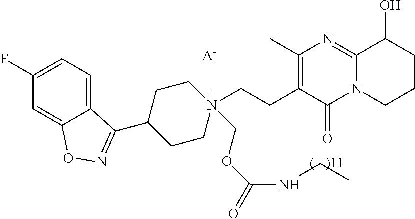 |
| 26 | 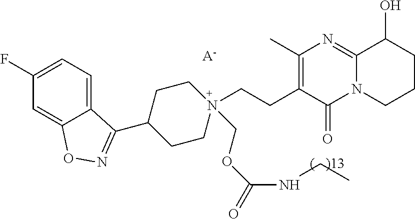 |
| 27 | 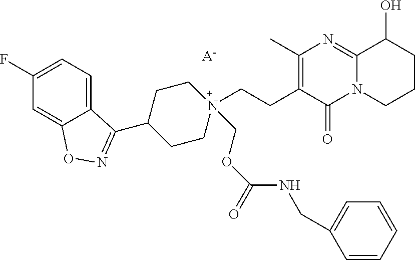 |
| 28 | 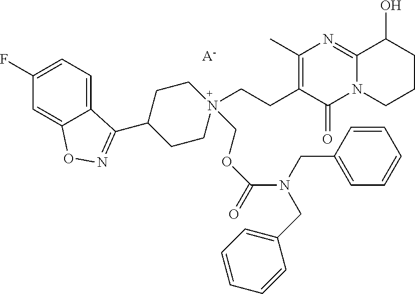 |
| 29 | 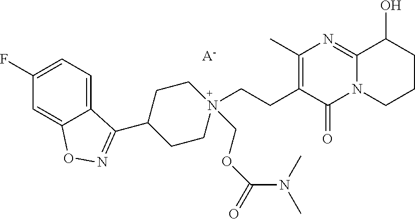 |
| 30 | 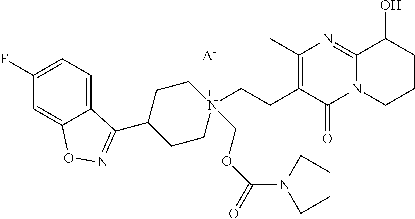 |
| 31 | 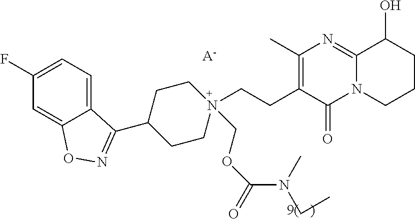 |
| 32 | 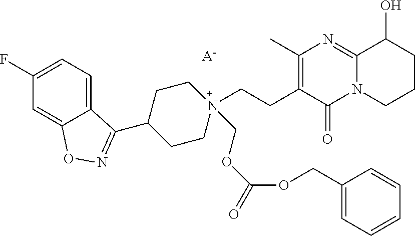 |
| 33 | 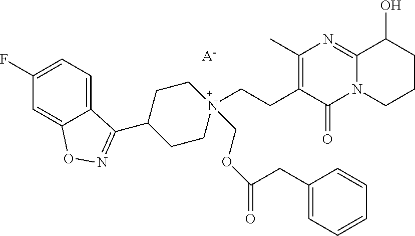 |
| 34 | 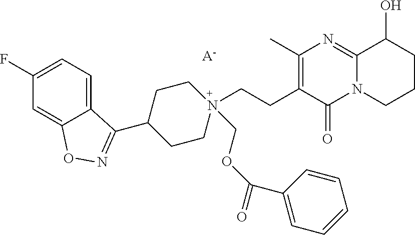 |
| 35 | 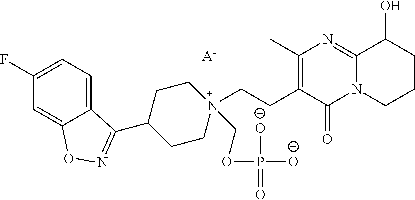 |
| 36 | 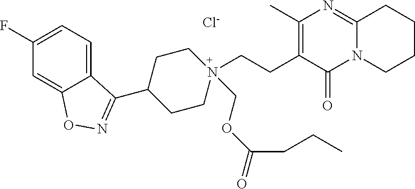 |
| 37 | 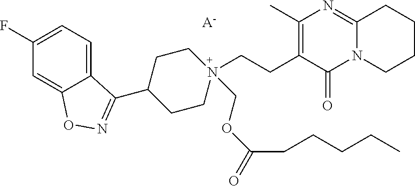 |
| 38 | 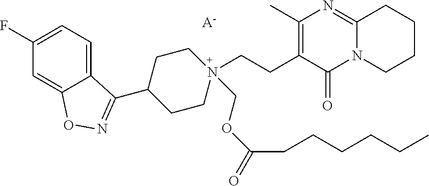 |
| 39 | 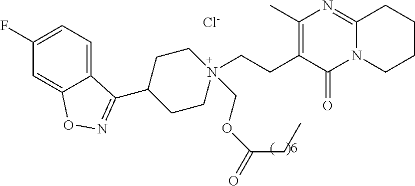 |
| 40 | 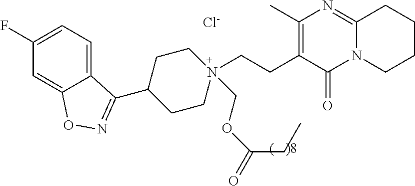 |
| 41 | 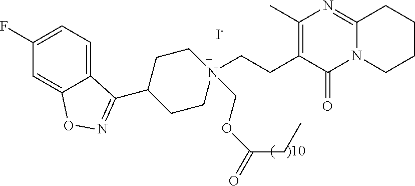 |
| 42 | 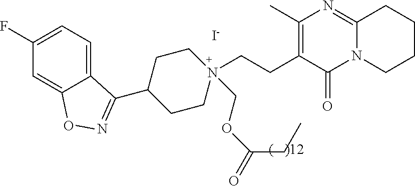 |
| 43 | 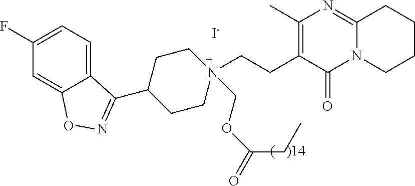 |
| 44 | 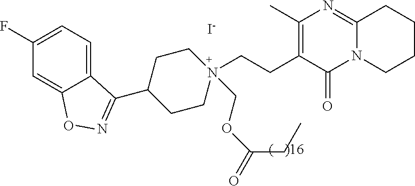 |
| 45 | 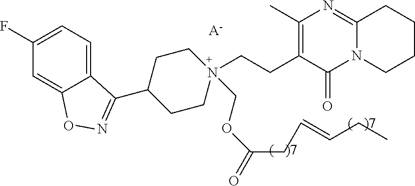 |
| 46 | 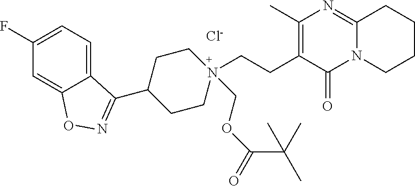 |
| 47 | 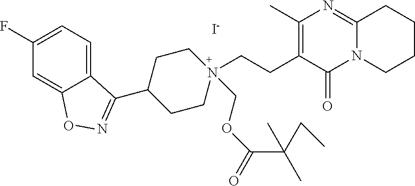 |
| 48 | 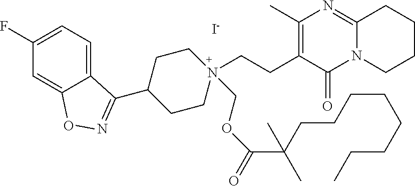 |
| 49 | 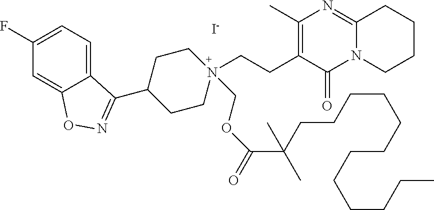 |
| 50 | 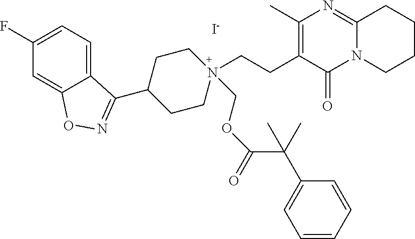 |
| 51 | 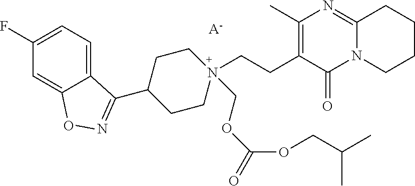 |
| 52 | 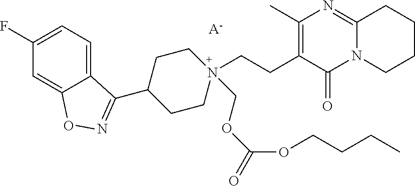 |
| 53 | 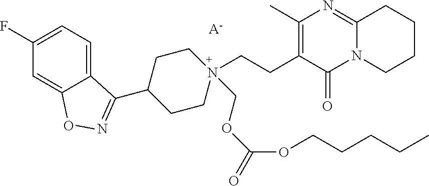 |
| 54 | 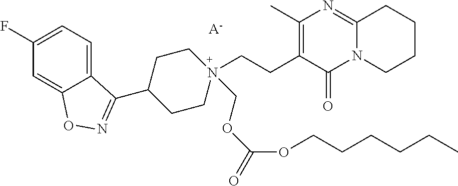 |
| 55 | 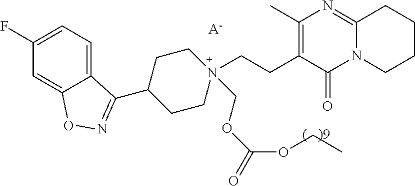 |
| 56 | 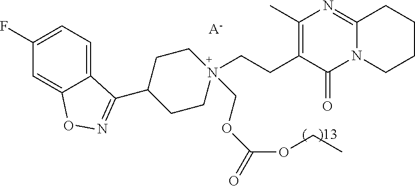 |
| 57 | 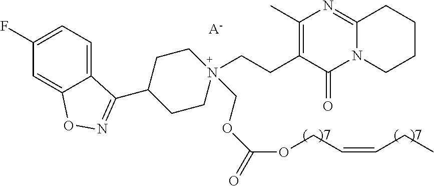 |
| 58 | 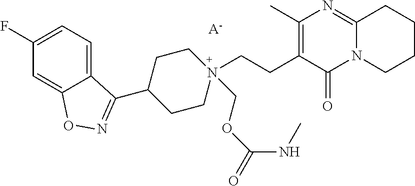 |
| 59 | 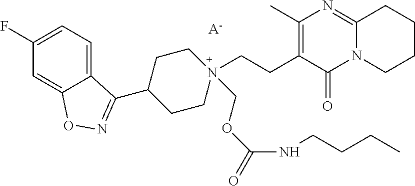 |
| 60 | 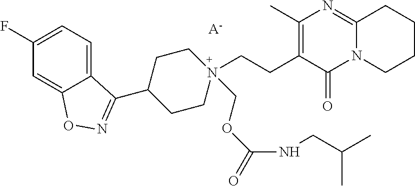 |
| 61 | 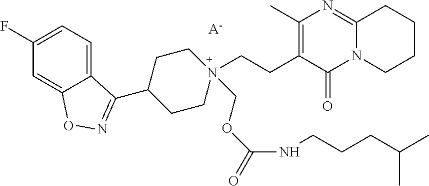 |
| 62 | 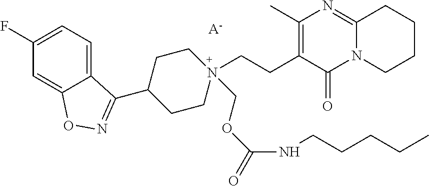 |
| 63 | 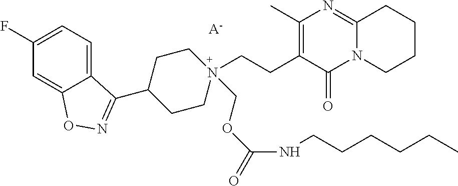 |
| 64 | 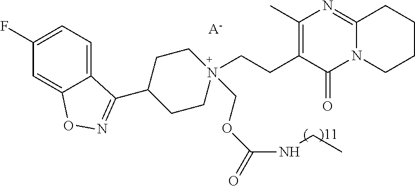 |
| 65 | 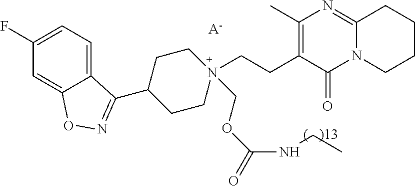 |
| 66 | 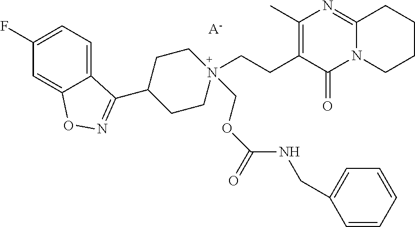 |
| 67 | 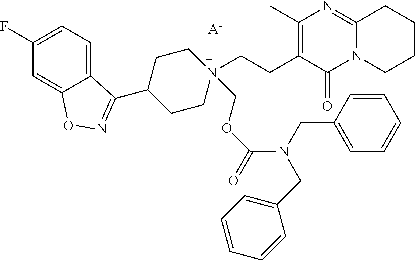 |
| 68 | 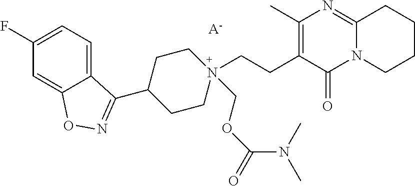 |
| 69 | 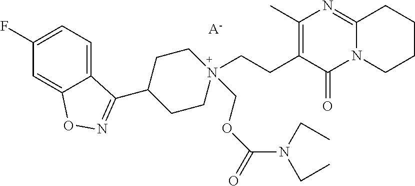 |
| 70 | 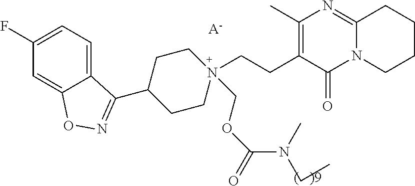 |
| 71 | 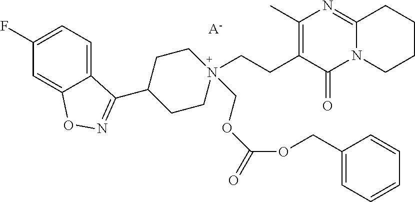 |
| 72 | 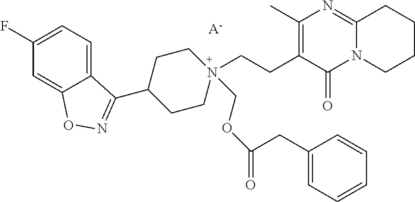 |
| 73 | 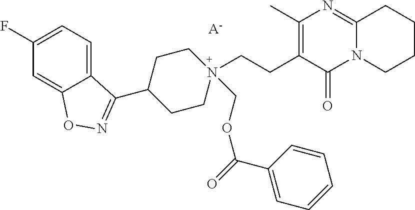 |
| 74 | 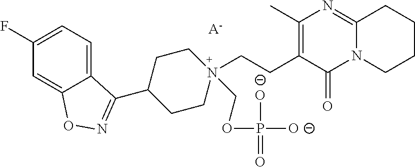 |
| 75 | 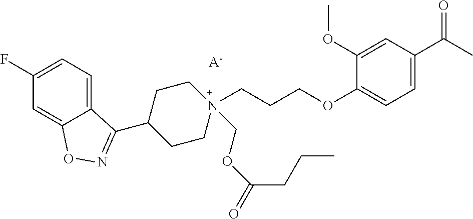 |
| 76 | 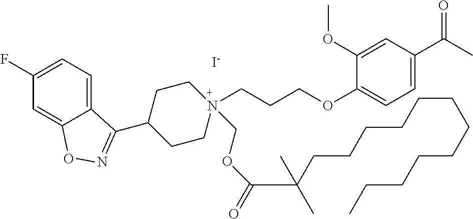 |
| 77 | 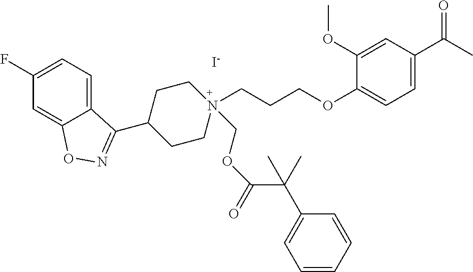 |
| 78 | 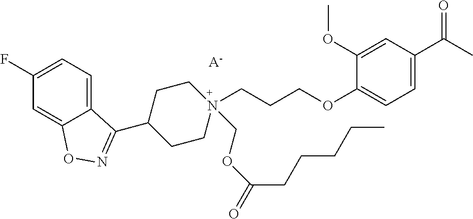 |
| 79 | 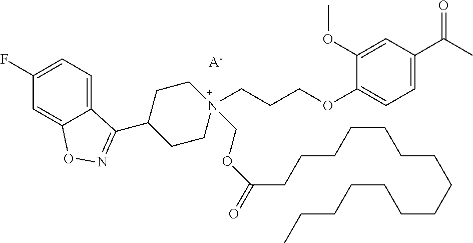 |
| 80 | 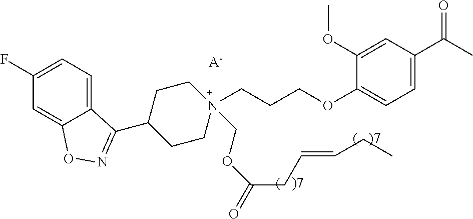 |
| 81 | 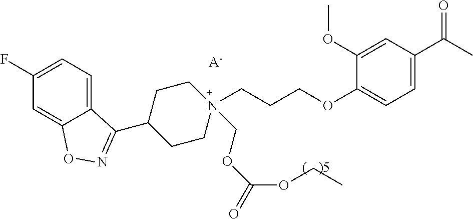 |
| 82 | 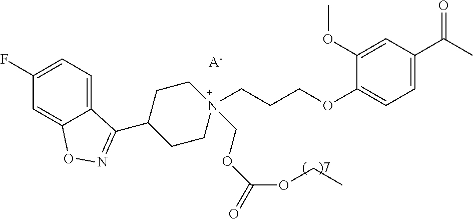 |
| 83 | 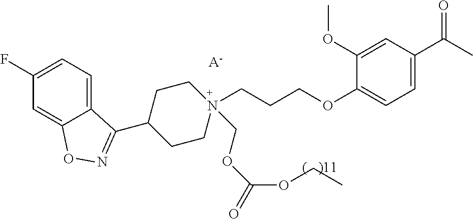 |
| 84 | 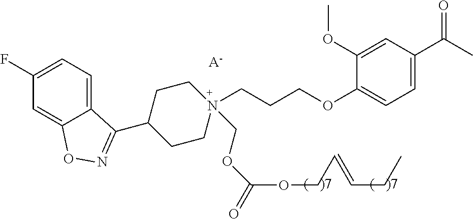 |
| 85 | 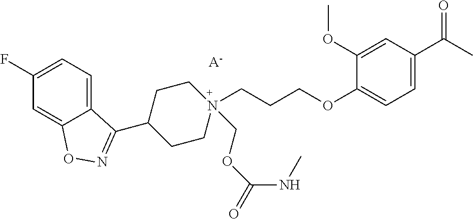 |
| 86 | 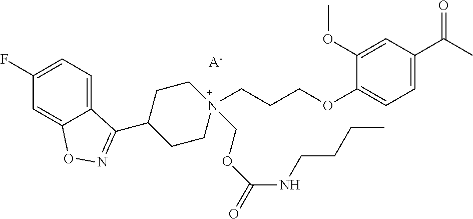 |
| 87 | 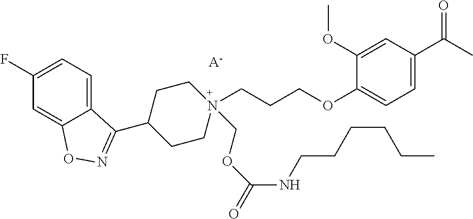 |
| 88 | 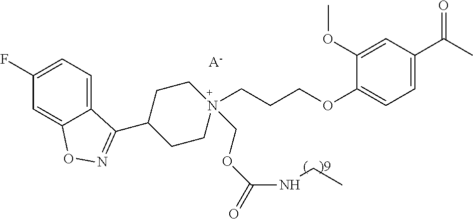 |
| 89 | 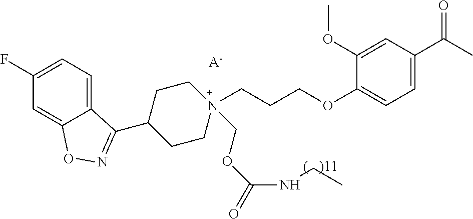 |
| 90 | 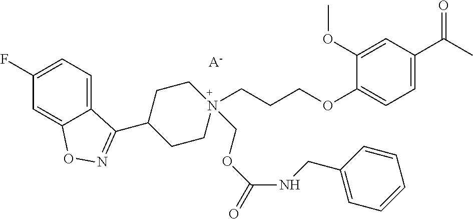 |
| 91 | 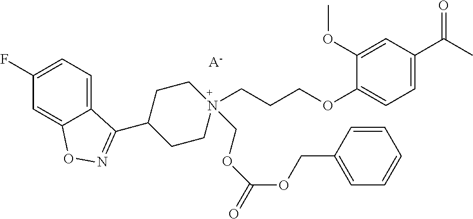 |
| 92 | 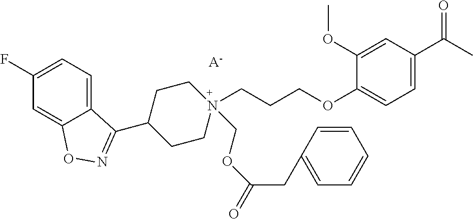 |
| 93 | 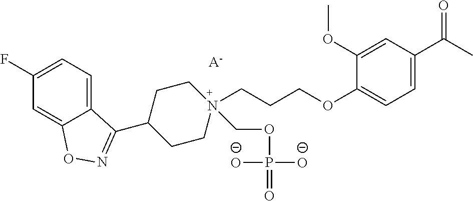 |
| 94 | 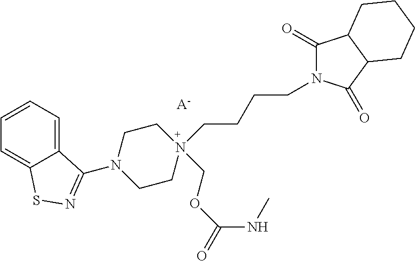 |
| 95 | 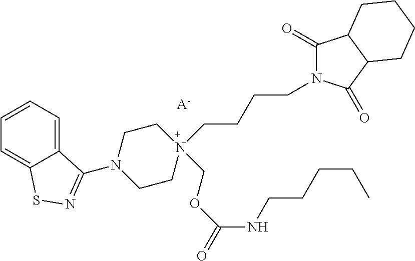 |
| 96 | 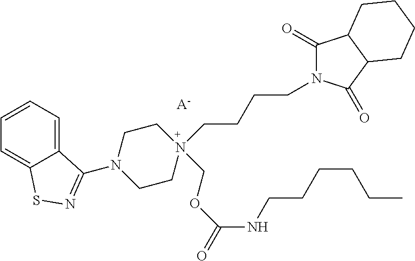 |
| 97 | 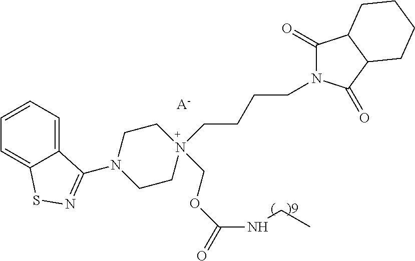 |
| 98 | 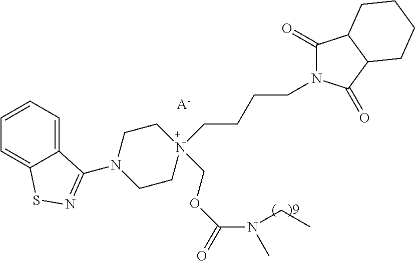 |
| 99 | 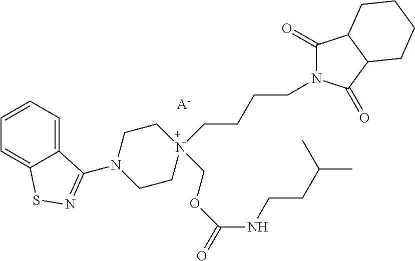 |
| 100 | 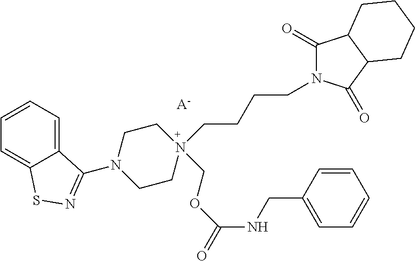 |
| 101 | 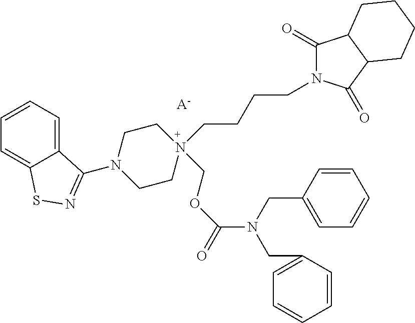 |
| 102 | 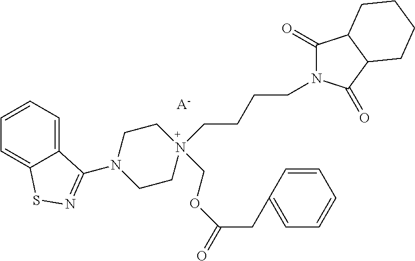 |
| 103 | 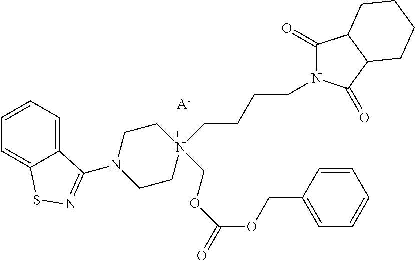 |
| 104 | 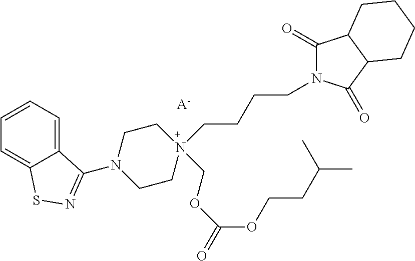 |
| 105 | 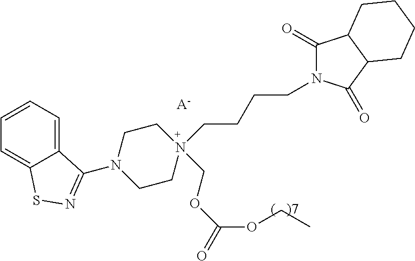 |
| 106 | 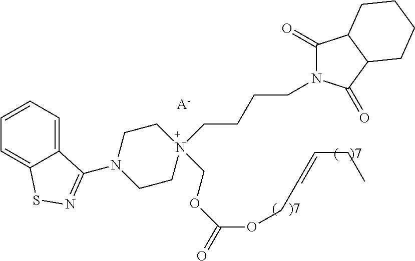 |
| 107 | 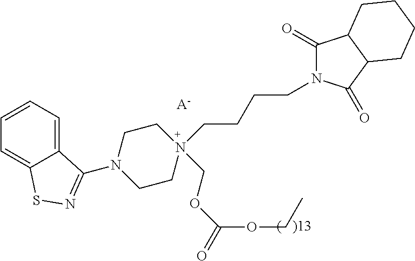 |
| 108 | 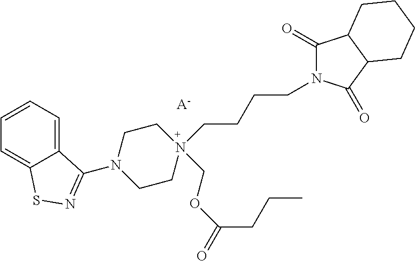 |
| 109 | 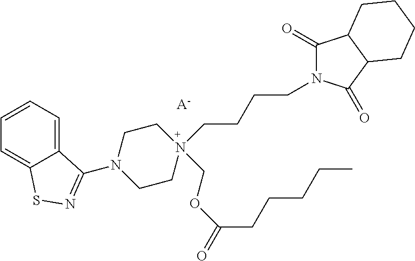 |
| 110 | 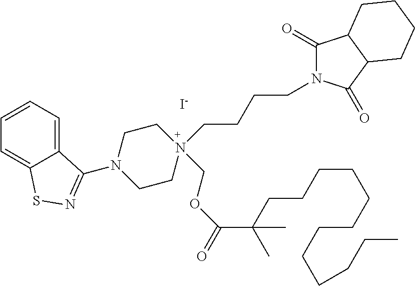 |
| 111 | 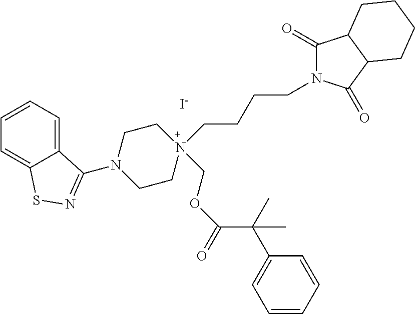 |
| 112 | 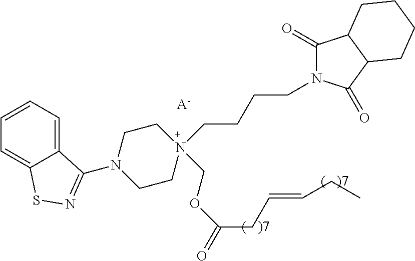 |
| 113 | 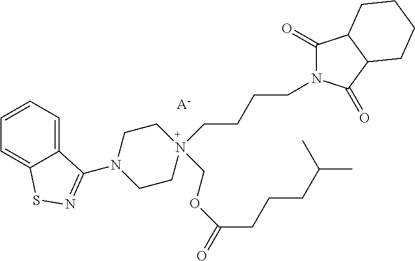 |
| 114 | 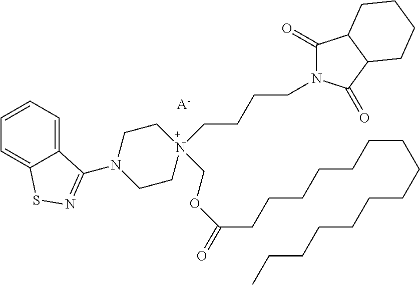 |
| 115 | 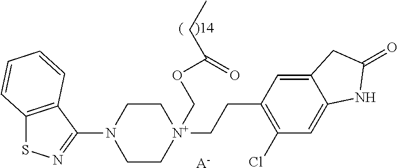 |
| 116 | 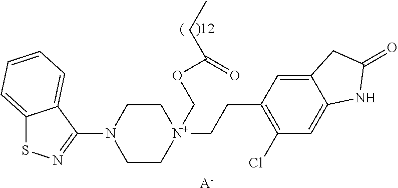 |
| 117 | 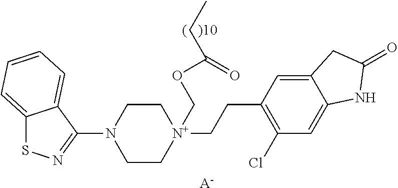 |
| 118 | 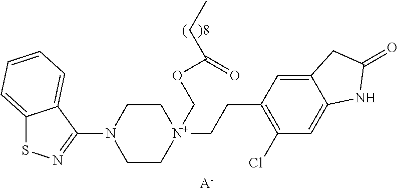 |
| 119 | 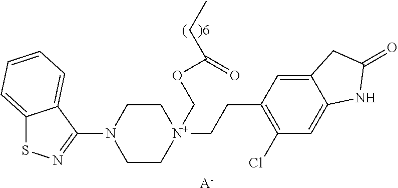 |
| 120 | 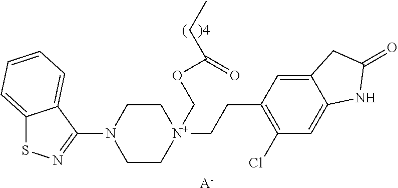 |
| 121 | 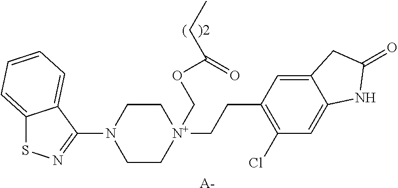 |
| 122 | 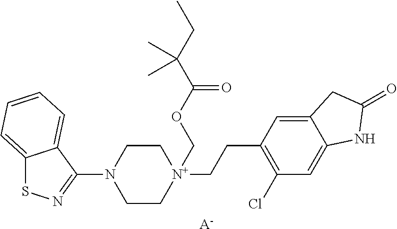 |
| 123 | 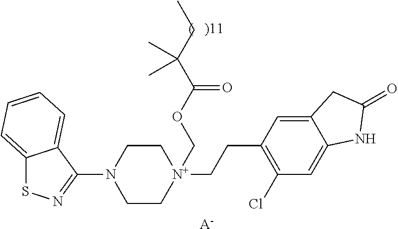 |
| 124 | 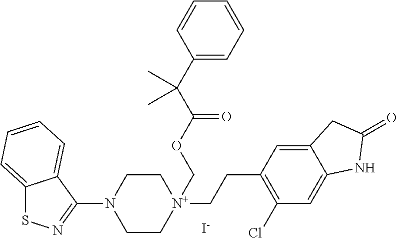 |
| 125 | 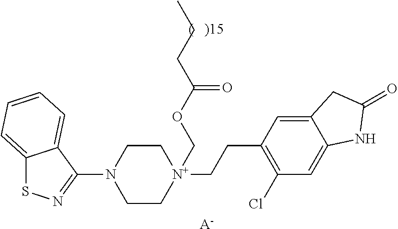 |
| 126 | 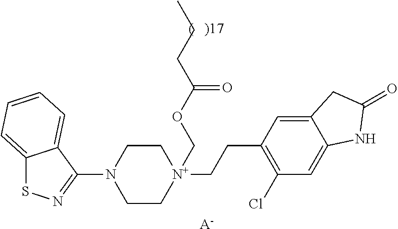 |
| 127 | 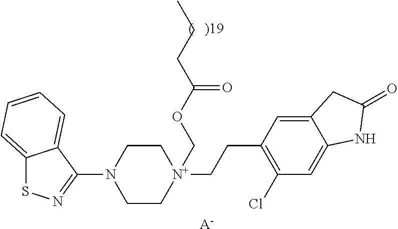 |
| 128 | 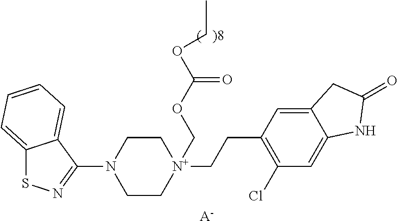 |
| 129 | 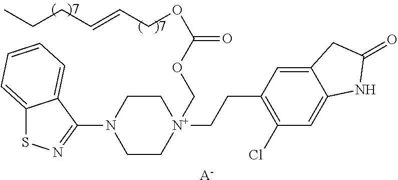 |
| 130 | 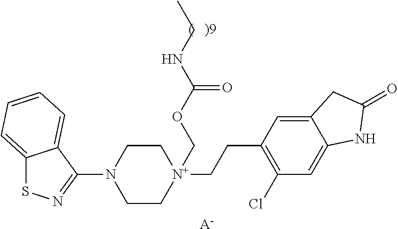 |
| 131 | 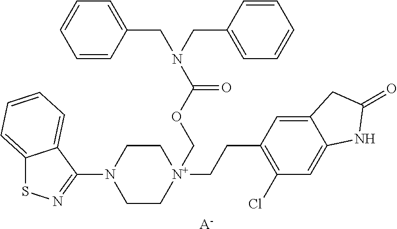 |
| 132 | 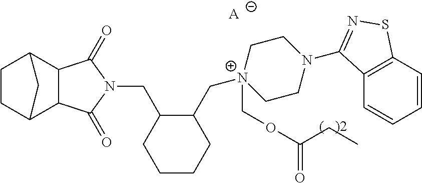 |
| 133 | 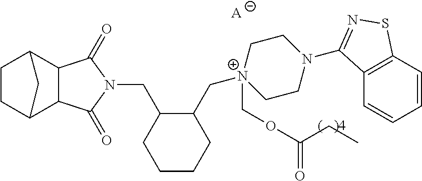 |
| 134 | 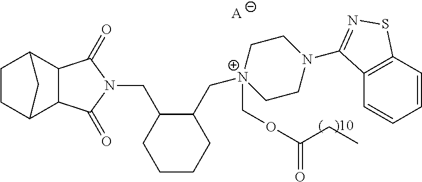 |
| 135 | 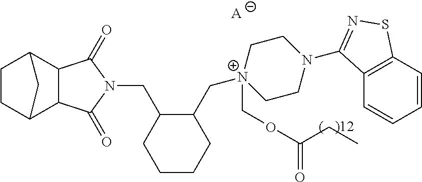 |
| 136 | 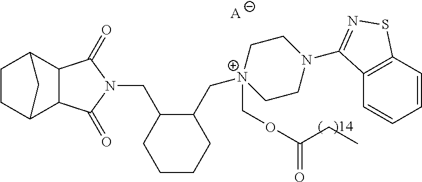 |
| 137 | 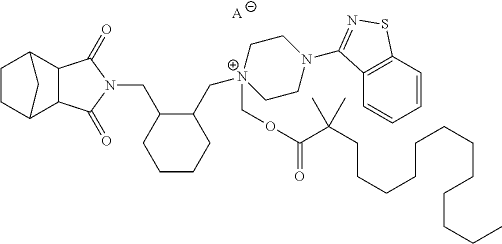 |
| 138 | 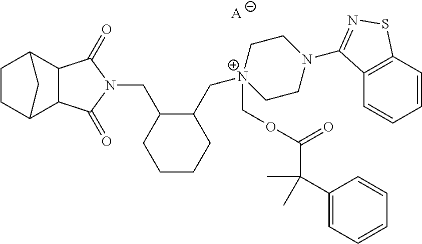 |
| 139 | 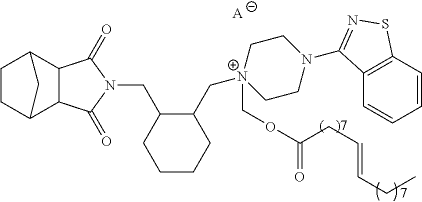 |
| 140 | 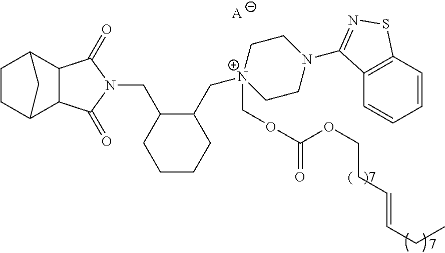 |
| 141 | 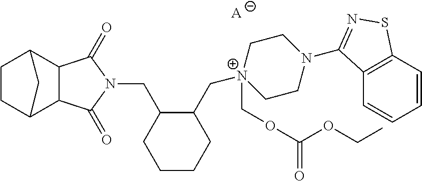 |
| 142 | 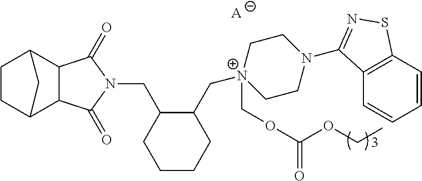 |
| 143 | 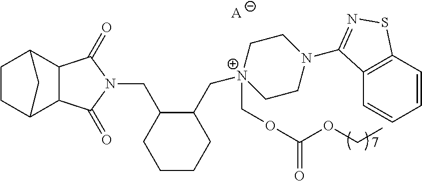 |
| 144 | 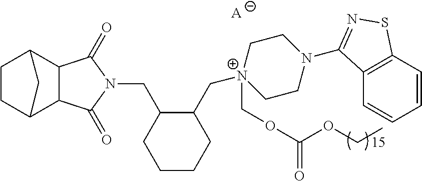 |
| 145 | 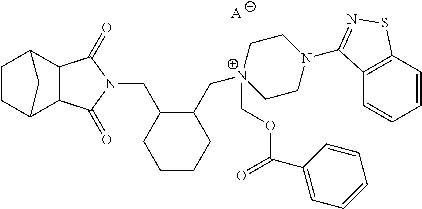 |
| 146 | 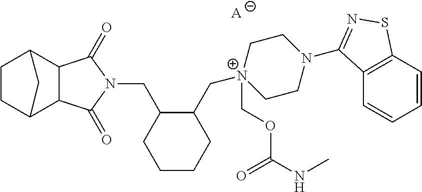 |
| 147 | 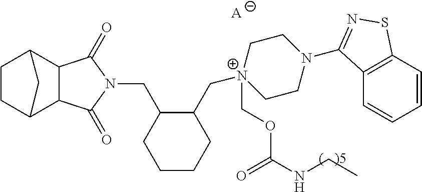 |
| 148 | 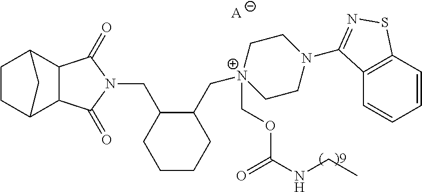 |
| 149 | 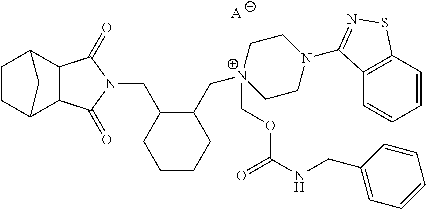 |
| 150 | 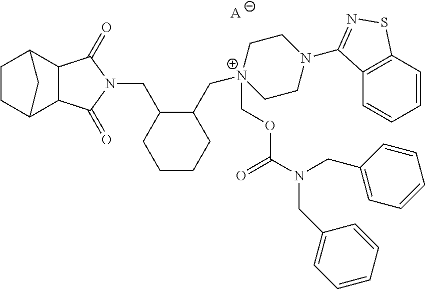 |
| 151 | 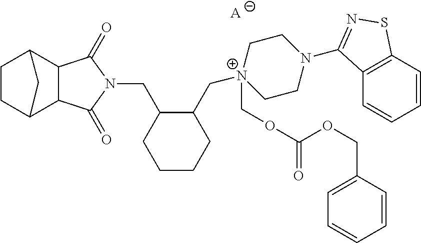 |
| 152 | 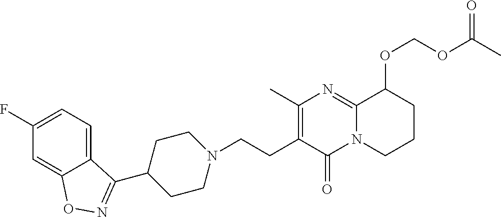 |
| 153 | 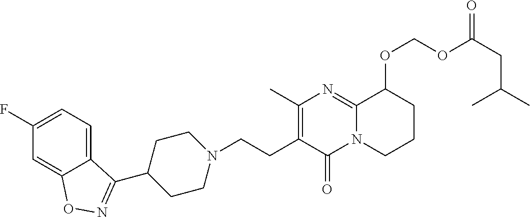 |
| 154 | 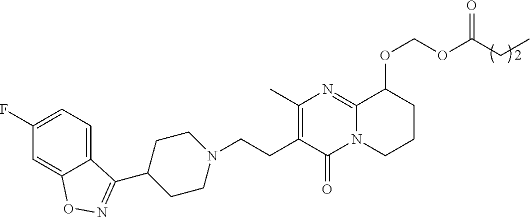 |
| 155 | 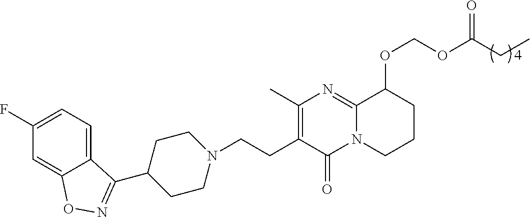 |
| 156 | 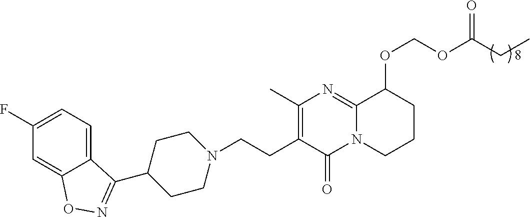 |
| 157 | 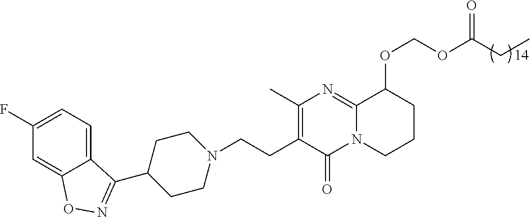 |
| 158 | 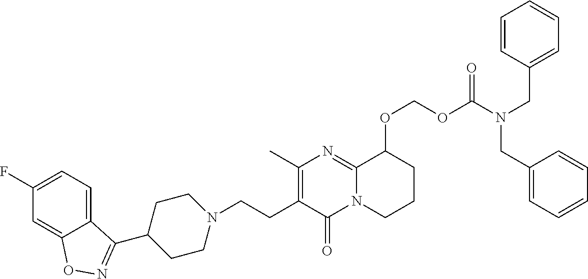 |
| 159 | 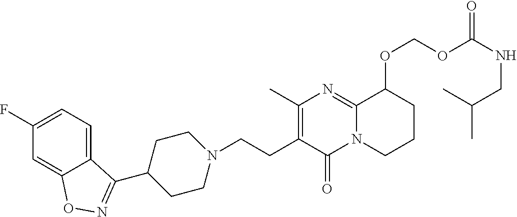 |
| 160 | 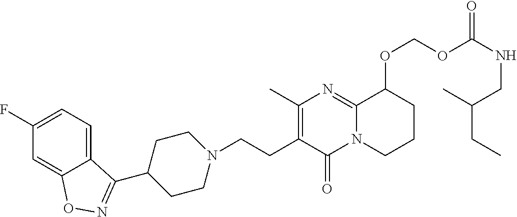 |
| 161 | 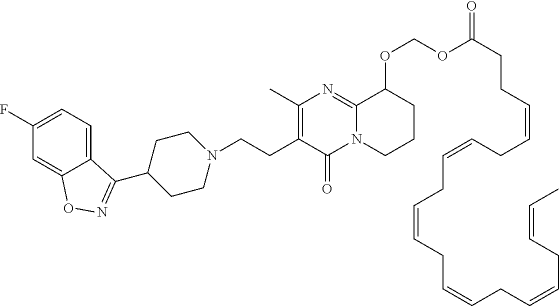 |
| 162 | 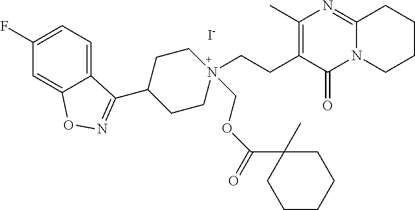 |
| 163 | 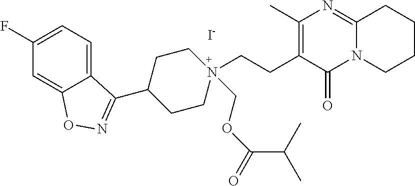 |
| 164 | 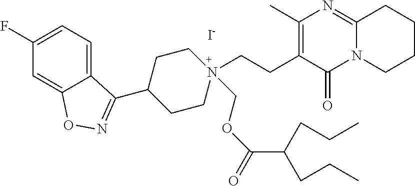 |
| 165 | 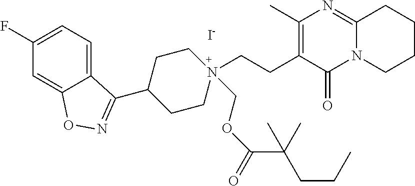 |
| 166 | 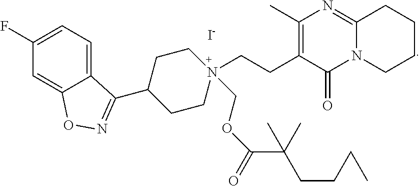 |

In another aspect of the invention, a general method to convert paliperidone, risperidone, iloperidone, lurasidone, perospirone, and ziprasidone to substituted tertiary amines is provided (Scheme 1).

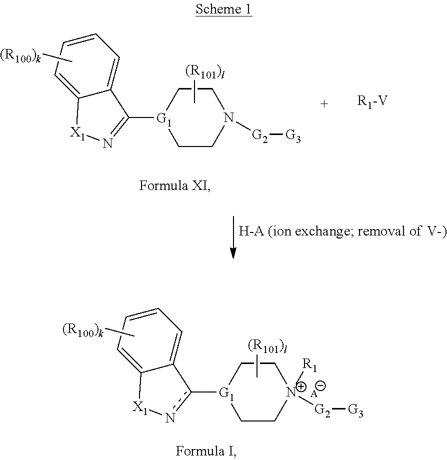

wherein k, l, R₁, X₁, R₁₀₀, R₁₀₁, G₁, G₂ and G₃ are as defined above; and,

V is a leaving group. In a preferred embodiment, V− is removed through ion exchange with a desired counterion, A−. A preferred counterion is chloride.

In one preferred embodiment, the prodrug compound of formulae I, III, V, VI, VII, VIII, IX and X further comprises a biocompatible delivery system for delivering the prodrug wherein the system is preferably capable of minimizing accelerated hydrolytic cleavage of the prodrug. Preferably the biocompatible delivery system is capable of minimizing hydrolytic cleavage by minimizing exposure of the prodrug to water and/or minimizing exposure to pH conditions deviating from the physiological range of pH (e.g. about 7). Preferred delivery systems include biocompatible polymeric matrix delivery systems comprising the prodrug and capable of minimizing diffusion of water into the matrix.

In another embodiment, the compounds of the invention that are quaternary amine containing salts such as compounds Formulas I, III, V, VI, VII, VIII, IX and X are less soluble at a reference pH than the parent drug from which they were derived. As used herein the term “reference pH” refers to the pH at which the aqueous solubility of a prodrug of the invention is compared to the aqueous solubility of the parent drug (not in prodrug form). Generally the reference pH is the pH at which the parent drug is essentially fully protonated. Typically, the reference pH is about 5 and is preferably in the range of 4-6 and is more preferably in the range of about pH 4 to about pH 8. Preferably, the aqueous solubility of a quaternary amine-containing prodrug compound of the invention at the reference pH is at least an order of magnitude lower than that of the aqueous solubility of the parent drug. In one embodiment, the quaternary amine-containing prodrug of the invention has a solubility of less than about 1 mg/ml, 0.5 mg/mL, 0.1 mg/mL, 0.01 mg/mL or 0.001 mg/mL at a reference pH, such as a pH of about 5.

Other embodiments of the invention are based on the unexpected discovery that the increased insolubility of the quaternary amine-containing prodrugs of the invention is independent of pH in aqueous media. One of the features of the prodrugs of Formulas Formulas I, III, V, VI, VII, VIII, IX and X of the invention is that they are less soluble than their parent, tertiary amine-containing drugs at a reference pH such as the pH wherein the parent drug (not in prodrug form) would generally be protonated (e.g. around pH 5.0), which feature contributes to the sustained release profile of the prodrug upon administration to a patient as compared to the parent tertiary amine containing drug when administered alone. However, it is known in the art that sustained release preparations of drugs of pH-dependent solubility are susceptible to changes in pH which can lead to changes in the behavior of the sustained release formulation such as the solubility of the drug in the formulation.

Sustained release drug formulations often contain higher amounts of drugs than immediate release formulations. Functionality and safety of a sustained release formulation are based on a reliable and controlled rate of drug release from the formulation over an extended period of time after administration. The drug release profile of a formulation often depends on the chemical environment of the sustained release formulation, for example, on pH, ionic strength and presence of solvents such as ethanol.

The relatively high amount of drug that is present in a sustained release formulation can, in some instances, harm a patient if the formulation releases the drug at a rate that is faster than the intended controlled release rate. If the formulation releases the drug at a rate that is slower than the intended controlled release rate, the therapeutic efficacy of the drug can be reduced.

In most cases, partial or total failure of a sustained release formulation results in a rapid release of the drug into the bloodstream. This rapid release is generally significantly faster than the intended sustained release of the drug from the formulation, and is sometimes referred to as “dose dumping.”

Dose dumping can create severe consequences for a patient, including permanent harm and even death. Examples of drugs that can be fatal if the therapeutically beneficial dose is exceeded, e.g., by dose dumping, include pain medications such as opioids, as well as other agents active in the central nervous system. In those situations where dose dumping may not be fatal, dose dumping may at least be responsible for the side effect of increased sedation of the patient.

The present invention solves the problem of dose dumping and its associated side effects including, but not limited to, increased sedation in a sustained release formulation by providing prodrugs that are quaternary amine-containing salts that maintain their reduced solubility and sustained release action in a manner which is independent of the pH of the environment in which the prodrug is administered. The pH-independent solubility of the quaternary amine-containing prodrugs of the invention is an important feature for drugs that are administered both orally and by injection. During oral administration, the prodrugs of the invention are exposed to a variety of pH conditions including very low pH in the stomach (e.g. pH 1-2) and then increased pH when crossing the intestinal walls into the bloodstream. During injection it has been observed that the pH at the injection site may also be lowered (e.g. below pH 6). [CRS 2009 Annual Meeting, Copenhagen Denmark, poster 242; Steen K H, Steen A E, Reeh P W; and article entitled “A dominant role of acid pH in inflammatory excitation and sensitization of nociceptors in rat skin, in vitro” in The Journal of Neuroscience (1995), 15: 3982-3989]. The pH of an injection site may be lowered for a short amount of time (1-2 hours), but the perturbation may be sufficient to dissolve a basic drug having pH-dependent solubility. In accordance with the invention, the reduced solubility of the prodrugs of the invention remains independent of any change in pH. In one preferred embodiment the reduced solubility of the prodrugs of the invention remains independent over a pH range of about pH 4 to about pH 8. More preferably the reduced solubility of the prodrugs of the invention remains independent over a pH range of about pH 3 to about pH 9. Most preferably, the reduced solubility of the prodrugs of the invention remains independent over a pH range of about pH 1.0 to about pH 10.

In addition, it is known that the stability of carboxyl ester linkages, such as those contemplated in the quaternary amine-containing prodrugs of the invention, is dependent on pH with optimum stability occurring at around pH 4-5. If injection site pH fluctuates to a value lower than neutral pH of 7.4, then the stability of the prodrug is increased relative to neutral pH. This stability increase further reduces the risk of early release of active drug from the compound, and thus avoids dose dumping by way of accelerated chemical cleavage of the prodrug.

Therefore the present invention further provides methods of pH-independent sustained release delivery of quaternary amine-containing prodrugs of the invention to a patient comprising administering a prodrug of Formulas I, III, V, VI, VII, VIII, IX and X, to the patient.

In a preferred embodiment, a compound of the invention provides sustained delivery of the parent drug over hours, days, weeks or months when administered parenterally to a subject. For example, the compounds can provide sustained delivery of the parent drug for up to 7, 15, 30, 60, 75 or 90 days or longer. Without being bound by theory, it is believed that the compounds of the invention form an insoluble depot upon parenteral administration, for example subcutaneous, intramuscular or intraperitoneal injection.

In another preferred embodiment, the prodrug of the invention provides sustained delivery of the parent drug when delivered orally. The prodrugs of the invention are generally stable to hydrolysis in the low pH of the stomach. Given that the solubility of the prodrugs of the invention is pH independent, crossing from the intestine having a low pH to the blood stream having a pH of around 7 will not cause the prodrugs to become soluble and release the full dose of free drug (dose dump). In a preferred embodiment, the orally delivered prodrugs further comprise a delivery system capable of enhancing sustained release and providing protection from enzymatic and chemical cleavage in the stomach and upper intestines. Additionally, such prodrug delivery system may comprise lipid-like features that facilitate uptake via lymph fluid, thus diverting prodrug from exposure to the liver on the way to the systemic circulation. This latter property can be advantageous for drugs that experience metabolism in the liver to metabolites that are undesirable due to inactivity and/or toxicity.

In one embodiment the invention provides methods of reducing the side effect of increased sedation in a patient as compared to sedation caused by administration of the parent drug of formula XI comprising administering a prodrug compound of the invention selected from Formulas I, III, V, VI, VII, VIII, IX and X.

### DEFINITIONS

Listed below are definitions of various terms used to describe this invention. These definitions apply to the terms as they are used throughout this specification and claims, unless otherwise limited in specific instances, either individually or as part of a larger group.

The term “acyl” refers to a carbonyl substituted with hydrogen, alkyl, partially saturated or fully saturated cycloalkyl, partially saturated or fully saturated heterocycle, aryl, or heteroaryl. For example, acyl includes groups such as (C₁-C₆) alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions. When indicated as being “optionally substituted”, the acyl group may be unsubstituted or optionally substituted with one or more substituents (typically, one to three substituents) independently selected from the group of substituents listed below in the definition for “substituted” or the alkyl, cycloalkyl, heterocycle, aryl and heteroaryl portion of the acyl group may be substituted as described above in the preferred and more preferred list of substituents, respectively.

The term “alkyl” is intended to include both branched and straight chain, substituted or unsubstituted, saturated aliphatic hydrocarbon radicals/groups having the specified number of carbons. Preferred alkyl groups comprise about 1 to about 24 carbon atoms (“C₁-C₂₄”) preferably about 7 to about 24 carbon atoms (“C₇-C₂₄”), preferably about 8 to about 24 carbon atoms (“C₈-C₂₄”), preferably about 9 to about 24 carbon atoms (“C₉-C₂₄”). Other preferred alkyl groups comprise at about 1 to about 8 carbon atoms (“C₁-C₈”) such as about 1 to about 6 carbon atoms (“C₁-C₆”), or such as about 1 to about 3 carbon atoms (“C₁-C₃”). Examples of C₁-C₆ alkyl radicals include, but are not limited to, methyl, ethyl, propyl, isopropyl, n-butyl, tert-butyl, n-pentyl, neopentyl and n-hexyl radicals.

The term “alkenyl” refers to linear or branched radicals having at least one carbon-carbon double bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkenyl radicals are “lower alkenyl” radicals having two to about ten carbon atoms (“C₂-C₁₀”) such as ethenyl, allyl, propenyl, butenyl and 4-methylbutenyl. Preferred lower alkenyl radicals include 2 to about 6 carbon atoms (“C₂-C₆”). The terms “alkenyl”, and “lower alkenyl”, embrace radicals having “cis” and “trans” orientations, or alternatively, “E” and “Z” orientations.

The term “alkynyl” refers to linear or branched radicals having at least one carbon-carbon triple bond. Such radicals preferably contain from about two to about twenty-four carbon atoms (“C₂-C₂₄”) preferably about 7 to about 24 carbon atoms (“C₇-C₂₄”), preferably about 8 to about 24 carbon atoms (“C₈-C₂₄”), and preferably about 9 to about 24 carbon atoms (“C₉-C₂₄”). Other preferred alkynyl radicals are “lower alkynyl” radicals having two to about ten carbon atoms such as propargyl, 1-propynyl, 2-propynyl, 1-butyne, 2-butynyl and 1-pentynyl. Preferred lower alkynyl radicals include 2 to about 6 carbon atoms (“C₂-C₆”).

The term “cycloalkyl” refers to saturated carbocyclic radicals having three to about twelve carbon atoms (“C₃-C₁₂”). The term “cycloalkyl” embraces saturated carbocyclic radicals having three to about twelve carbon atoms. Examples of such radicals include cyclopropyl, cyclobutyl, cyclopentyl and cyclohexyl.

The term “cycloalkenyl” refers to partially unsaturated carbocyclic radicals having three to twelve carbon atoms. Cycloalkenyl radicals that are partially unsaturated carbocyclic radicals that contain two double bonds (that may or may not be conjugated) can be called “cycloalkyldienyl”. More preferred cycloalkenyl radicals are “lower cycloalkenyl” radicals having four to about eight carbon atoms. Examples of such radicals include cyclobutenyl, cyclopentenyl and cyclohexenyl.

The term “alkylene,” as used herein, refers to a divalent group derived from a straight-chain or branched saturated hydrocarbon chain having the specified number of carbons atoms. Examples of alkylene groups include, but are not limited to, ethylene, propylene, butylene, 3-methyl-pentylene, and 5-ethyl-hexylene.

The term “alkenylene,” as used herein, denotes a divalent group derived from a straight-chain or branched hydrocarbon moiety containing the specified number of carbon atoms having at least one carbon-carbon double bond. Alkenylene groups include, but are not limited to, for example, ethenylene, 2-propenylene, 2-butenylene, 1-methyl-2-buten-1-ylene, and the like.

The term “alkynylene,” as used herein, denotes a divalent group derived from a straight-chain or branched hydrocarbon moiety containing the specified number of carbon atoms having at least one carbon-carbon triple bond. Representative alkynylene groups include, but are not limited to, for example, propynylene, 1-butynylene, 2-methyl-3-hexynylene, and the like.

The term “alkoxy” refers to linear or branched oxy-containing radicals each having alkyl portions of one to about twenty-four carbon atoms or, preferably, one to about twelve carbon atoms. More preferred alkoxy radicals are “lower alkoxy” radicals having one to about ten carbon atoms and more preferably having one to about eight carbon atoms. Examples of such radicals include methoxy, ethoxy, propoxy, butoxy and tert-butoxy.

The term “alkoxyalkyl” refers to alkyl radicals having one or more alkoxy radicals attached to the alkyl radical, that is, to form monoalkoxyalkyl and dialkoxyalkyl radicals.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl.

The terms “heterocyclyl”, “heterocycle” “heterocyclic” or “heterocyclo” refer to saturated, partially unsaturated and unsaturated heteroatom-containing ring-shaped radicals, which can also be called “heterocyclyl”, “heterocycloalkenyl” and “heteroaryl” correspondingly, where the heteroatoms may be selected from nitrogen, sulfur and oxygen. Examples of saturated heterocyclyl radicals include saturated 3 to 6-membered heteromonocyclic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. Heterocyclyl radicals may include a pentavalent nitrogen, such as in tetrazolium and pyridinium radicals. The term “heterocycle” also embraces radicals where heterocyclyl radicals are fused with aryl or cycloalkyl radicals. Examples of such fused bicyclic radicals include benzofuran, benzothiophene, and the like.

The term “heteroaryl” refers to unsaturated aromatic heterocyclyl radicals. Examples of heteroaryl radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like.

The term “heterocycloalkyl” refers to heterocyclo-substituted alkyl radicals. More preferred heterocycloalkyl radicals are “lower heterocycloalkyl” radicals having one to six carbon atoms in the heterocyclo radical.

The term “alkylthio” refers to radicals containing a linear or branched alkyl radical, of one to about ten carbon atoms attached to a divalent sulfur atom. Preferred alkylthio radicals have alkyl radicals of one to about twenty-four carbon atoms or, preferably, one to about twelve carbon atoms. More preferred alkylthio radicals have alkyl radicals which are “lower alkylthio” radicals having one to about ten carbon atoms. Most preferred are alkylthio radicals having lower alkyl radicals of one to about eight carbon atoms. Examples of such lower alkylthio radicals include methylthio, ethylthio, propylthio, butylthio and hexylthio.

The terms “aralkyl” or “arylalkyl” refer to aryl-substituted alkyl radicals such as benzyl, diphenylmethyl, triphenylmethyl, phenylethyl, and diphenylethyl.

The term “aryloxy” refers to aryl radicals attached through an oxygen atom to other radicals.

The terms “aralkoxy” or “arylalkoxy” refer to aralkyl radicals attached through an oxygen atom to other radicals.

The term “aminoalkyl” refers to alkyl radicals substituted with amino radicals. Preferred aminoalkyl radicals have alkyl radicals having about one to about twenty-four carbon atoms or, preferably, one to about twelve carbon atoms. More preferred aminoalkyl radicals are “lower aminoalkyl” that have alkyl radicals having one to about ten carbon atoms. Most preferred are aminoalkyl radicals having lower alkyl radicals having one to eight carbon atoms. Examples of such radicals include aminomethyl, aminoethyl, and the like.

The term “alkylamino” denotes amino groups which are substituted with one or two alkyl radicals. Preferred alkylamino radicals have alkyl radicals having about one to about twenty carbon atoms or, preferably, one to about twelve carbon atoms. More preferred alkylamino radicals are “lower alkylamino” that have alkyl radicals having one to about ten carbon atoms. Most preferred are alkylamino radicals having lower alkyl radicals having one to about eight carbon atoms. Suitable lower alkylamino may be monosubstituted N-alkylamino or disubstituted N,N-alkylamino, such as N-methylamino, N-ethylamino, N,N-dimethylamino, N,N-diethylamino or the like.

The term “substituted” refers to the replacement of one or more hydrogen radicals in a given structure with the radical of a specified substituent including, but not limited to: halo, alkyl, alkenyl, alkynyl, aryl, heterocyclyl, thiol, alkylthio, arylthio, alkylthioalkyl, arylthioalkyl, alkylsulfonyl, alkylsulfonylalkyl, arylsulfonylalkyl, alkoxy, aryloxy, aralkoxy, aminocarbonyl, alkylaminocarbonyl, arylaminocarbonyl, alkoxycarbonyl, aryloxycarbonyl, haloalkyl, amino, trifluoromethyl, cyano, nitro, alkylamino, arylamino, alkylaminoalkyl, arylaminoalkyl, aminoalkylamino, hydroxy, alkoxyalkyl, carboxyalkyl, alkoxycarbonylalkyl, aminocarbonylalkyl, acyl, aralkoxycarbonyl, carboxylic acid, sulfonic acid, sulfonyl, phosphonic acid, aryl, heteroaryl, heterocyclic, and aliphatic. It is understood that the substituent may be further substituted.

The terms “halogen” or “halo” as used herein, refers to an atom selected from fluorine, chlorine, bromine and iodine.

The terms “compound” “drug”, and “prodrug” as used herein all include pharmaceutically acceptable salts, solvates, hydrates, polymorphs, enantiomers, diastereoisomers, racemates and the like of the compounds, drugs and prodrugs having the formulas as set forth herein.

Substituents indicated as attached through variable points of attachments can be attached to any available position on the ring structure.

For simplicity, chemical moieties that are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

As used herein, the term “effective amount of the subject compounds,” with respect to the subject method of treatment, refers to an amount of the subject compound which, when delivered as part of desired dose regimen, brings about management of the disease or disorder to clinically acceptable standards.

“Treatment” or “treating” refers to an approach for obtaining beneficial or desired clinical results in a patient. For purposes of this invention, beneficial or desired clinical results include, but are not limited to, one or more of the following: alleviation of symptoms, diminishment of extent of a disease, stabilization (i.e., not worsening) of a state of disease, preventing spread (i.e., metastasis) of disease, preventing occurrence or recurrence of disease, delay or slowing of disease progression, amelioration of the disease state, and remission (whether partial or total).

The neurological and psychiatric disorders include, but are not limited to, disorders such as cerebral deficit subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, cerebral deficits secondary to prolonged status epilepticus, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, social phobia, obsessive compulsive disorder, and post-traumatic stress disorder (PTSD)), mood disorders (including depression, mania, bipolar disorders), circadian rhythm disorders (including jet lag and shift work), trigeminal neuralgia, hearing loss, tinnitus, macular degeneration of the eye, emesis, brain edema, pain (including acute and chronic pain states, severe pain, intractable pain, neuropathic pain, inflammatory pain, and post-traumatic pain), tardive dyskinesia, sleep disorders (including narcolepsy), attention deficit/hyperactivity disorder, eating disorders, and conduct disorder.

The compounds of the invention can be prepared as acid addition salts. Preferably, the acid is a pharmaceutically acceptable acid. Such acids are described in Stahl, P. H. and Wermuth, C. G. (eds.), Handbook of Pharmaceutical Salts: Properties, Selection and Use, Wiley VCH (2008). Pharmaceutically acceptable acids include acetic acid, dichloroacetic acid, adipic acid, alginic acid, L-ascorbic acid, L-aspartic acid, benzenesulfonic acid, 4-acetamidobenzoic acid, benzoic acid, p-bromophenylsulfonic acid; (+)-camphoric acid, (+)-camphor-10-sulfonic acid, capric acid, caproic acid, caprylic acid, carbonic acid, cinnamic acid, cyclamic acid, dodecylsulfonic acid, ethane-1,2-disulfonic acid, ethanesulfonic acid, 2-hydroxyethanesulfonic acid, sulfuric acid, boric acid, citric acid, formic acid, fumaric acid, galactaric acid, gentisic acid, D-glucoheptonic acid, D-gluconic acid, D-glucuronic acid, glutamic acid, glutaric acid, 2-oxoglutaric acid, glycerophosphoric acid, glycolic acid, hippuric acid, hydrochloric acid, hydrobromic acid, hydroiodic acid, isobutyric acid, DL-lactic acid, lactobionic acid, lauric acid, maleic acid, (−)-L-malic acid, malonic acid, DL-mandelic acid, methanesulfonic acid, naphthalene-1,5-disulfonic acid, naphthalene-2-sulfonic acid, 1-hydroxy-2-naphthoic acid, nicotinic acid, nitric acid, oleic acid, orotic acid, oxalic acid, palmitic acid, pamoic acid, phosphoric acid, propionic acid, (−)-L-pyroglutamic acid, salicyclic acid, 4-aminosalicyclic acid, sebacic acid, stearic acid, succininc acid, (+)-L-tartaric acid, thiocyanic acid, p-toluenesulfonic acid, and undecylenic acid.

The term “pharmaceutically acceptable anion” as used herein, refers to the conjugate base of a pharmaceutically acceptable acid. Such anions include the conjugate base of any the acids set forth above. Preferred pharmaceutically acceptable anions include acetate, bromide, camsylate, chloride, formate, fumarate, iodide, malate, maleate, mesylate, nitrate, oxalate, phosphate, sulfate, tartrate, thiocyanate and tosylate.

As used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The synthesized compounds can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds described herein contain one or more asymmetric centers and thus give rise to enantiomers, diastereomers, and other stereoisomeric forms that may be defined, in terms of absolute stereochemistry, as (R)- or (S)-, or as (D)- or (L)- for amino acids and sugars. The present invention is meant to include all such possible isomers, as well as their racemic and optically pure forms. Optical isomers may be prepared from their respective optically active precursors by the procedures described above, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981). When the compounds described herein contain olefinic double bonds, other unsaturation, or other centers of geometric asymmetry, and unless specified otherwise, it is intended that the compounds include both E and Z geometric isomers and/or cis- and trans-isomers. Likewise, all tautomeric forms are also intended to be included. The configuration of any carbon-carbon double bond appearing herein is selected for convenience only and is not intended to designate a particular configuration unless the text so states; thus a carbon-carbon double bond or carbon-heteroatom double bond depicted arbitrarily herein as trans may be cis, trans, or a mixture of the two in any proportion.

In certain compounds of the invention, the quaternized nitrogen atom is a chiral center and both enantiomers are dealkylated in vivo to yield the parent drug. Such compounds can be formulated and used as a racemic mixture or as a composition having a single enantiomer or an enantiomeric excess of one enantiomer. In certain compounds the parent drug, such as asenapine, is chiral and can be used as a racemic mixture. For such a racemic mixture, quaternization of the nitrogen atom produces an additional chiral center and up to four stereoisomers. Such compounds can be formulated and used as a mixture of four stereoisomers. Alternatively, the diastereomers are separated to yield pairs of enantiomers, and a racemic mixture of one pair of enantiomers is formulated and used. In another embodiment, a single stereoisomer is formulated and used. Unless otherwise stated, the structural formula of a compound herein is intend to represent all enantiomers, racemates and diastereomers of that compound.

### Pharmaceutical Compositions

The pharmaceutical compositions of the present invention comprise a therapeutically effective amount of a compound of the present invention formulated together with one or more pharmaceutically acceptable carriers or excipients.

As used herein, the term “pharmaceutically acceptable carrier or excipient” means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; cyclodextrins such as alpha- (α), beta- (β) and gamma- (γ) cyclodextrins; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, dimethylacetamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable suspension or emulsion, such as INTRALIPID®, LIPOSYN® or Omegaven, or solution in a nontoxic parenterally acceptable diluent or solvent, for example, as a solution in 1,3-butanediol. INTRALIPID® is an intravenous fat emulsion containing 10-30% soybean oil, 1-10% egg yolk phospholipids, 1-10% glycerin and water. LIPOSYN® is also an intravenous fat emulsion containing 2-15% safflower oil, 2-15% soybean oil, 0.5-5% egg phosphatides 1-10% glycerin and water. OMEGAVEN® is an emulsion for infusion containing about 5-25% fish oil, 0.5-10% egg phosphatides, 1-10% glycerin and water. Among the acceptable vehicles and solvents that may be employed are water, Ringer's solution, USP, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissues.

In one preferred embodiment, the formulation provides a sustained release delivery system that is capable of minimizing the exposure of the prodrug to water. This can be accomplished by formulating the prodrug with a sustained release delivery system that is a polymeric matrix capable of minimizing the diffusion of water into the matrix. Suitable polymers comprising the matrix include polylactide (PLA) polymers and the lactide-co-glycolide (PLGA) co-polymers as described earlier. Other suitable polymers include tyrosinamide polymers (TyRx), as well as other biocompatible polymers.

Alternatively, the sustained release delivery system may comprise poly-anionic molecules or resins that are suitable for injection or oral delivery. Suitable polyanionic molecules include cyclodextrins and polysulfonates formulated to form a poorly soluble mass that minimizes exposure of the prodrug to water and from which the prodrug slowly leaves. Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition of the invention is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to VanDevanter et al., U.S. Pat. No. 5,508,269 to Smith et al., and WO 98/43650 by Montgomery, all of which are incorporated herein by reference). A discussion of pulmonary delivery of antibiotics is also found in U.S. Pat. No. 6,014,969, incorporated herein by reference.

By a “therapeutically effective amount” of a prodrug compound of the invention is meant an amount of the compound which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect).

In accordance with the invention, the therapeutically effective amount of a prodrug of the invention is typically based on the target therapeutic amount of the tertiary-amine containing parent drug. Information regarding dosing and frequency of dosing is readily available for many tertiary amine-containing parent drugs and the target therapeutic amount can be calculated for each prodrug of the invention. In accordance with the invention, the same dose of a prodrug of the invention provides a longer duration of therapeutic effect as compared to the parent drug. Thus if a single dose of the parent drug provides 12 hours of therapeutic effectiveness, a prodrug of that same parent drug in accordance with the invention that provides therapeutic effectiveness for greater than 12 hours will be considered to achieve a “sustained release” profile.

The precise dose of a prodrug of the invention depends upon several factors including the nature and dose of the parent drug and the chemical characteristics of the prodrug moiety linked to the parent drug. Ultimately, the effective dose and dose frequency of a prodrug of the invention will be decided by the attending physician within the scope of sound medical judgment. The specific therapeutically effective dose level and dose frequency for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or contemporaneously with the specific compound employed; and like factors well known in the medical arts.

### EXAMPLES

The compounds and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. General methodology for the preparation of paliperidone-, risperidone-, iloperidone-, perospirone-, and ziprasidone-related compounds can be found in the following patents: U.S. Pat. No. 5,158,952, U.S. Pat. No. 4,804,663, U.S. RE39198, US 2007/0254887 A1, U.S. Pat. No. 5,312,925.

### Example 1

### Risperidone

### Synthesis of Compound 36 (RSP Butyrate Chloride)

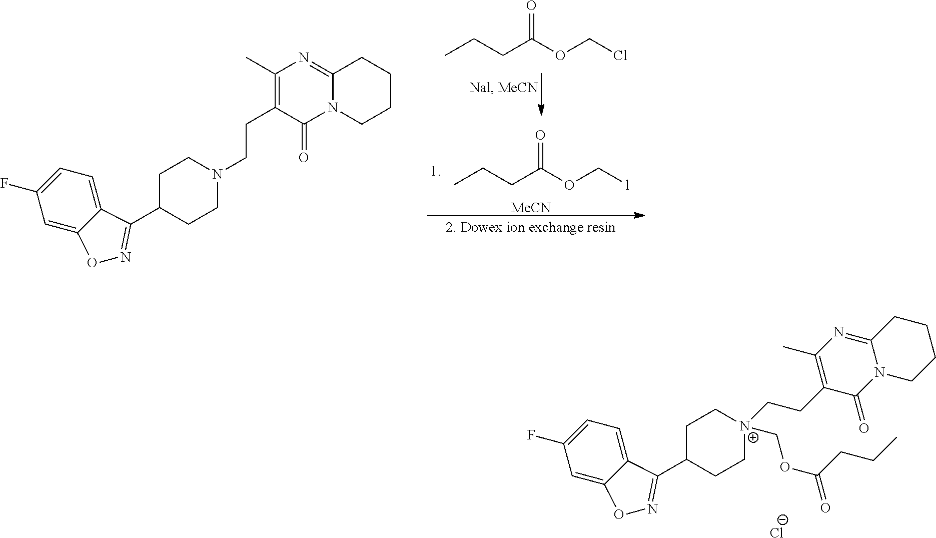

Step A: Synthesis of iodomethylbutyrate: To a solution of chloromethyl butyrate (6.11 g, 44.7 mmol) in acetonitrile (60 mL) was added sodium iodide (20.12 g, 134.2 mmol). The flask was covered in tin foil and stirred overnight at 25° C. The reaction mixture was partitioned between dichloromethane (200 mL) and water (100 mL). The aqueous layer was extracted with dichloromethane (2×100 mL). The combined organics were washed with aqueous saturated NaHCO₃ (100 mL), 5% aqueous sodium sulfite solution (100 mL) and brine (2×100 mL) then dried (MgSO₄) and concentrated to give iodomethyl butyrate (8.19 g, 80%). The iodide is used crude in the next reaction. ¹H-NMR (CDCl₃) δ 5.89 (2H, s), 2.31 (2H, t), 1.67 (2H, sextet), 0.95 (3H, t).

Step B: Synthesis of Compound 36: Iodomethyl butyrate (12 g, 52.6 mmol) and risperidone (5.4 g, 13.2 mmol) were stirred together in acetonitrile (100 mL) at 25° C. overnight (not all in solution). After stirring overnight the reaction was all completely dissolved and the reaction mixture concentrated to give a yellow oil which was triturated with diethyl ether (Et₂O) to remove aliphatic impurities. A pale yellow solid was obtained which was filtered and dried. The solid was a mixture of 2 conformers.

The solid was triturated twice with tetrahydrofuran (THF) to give one of the conformers (2.73 g). This was then passed through Dowex 1×8, 50-100 mesh, ion exchange resin eluting with de-ionized water to give the chloride which was triturated with Et₂O to give the chloride salt as a white solid (2.17 g).

¹H-NMR (CDCl₃) δ 7.95 (1H, dd), 7.22 (1H, dd), 7.11 (1H, dt), 6.03 (2H, s), 4.79 (2H, br t), 4.09 (1H, br s), 3.90-3.78 (4H, m), 3.59-3.54 (2H, m), 2.98-2.88 (4H, m), 2.59-2.39 (4H, m), 2.33 (3H, s), 2.04-1.88 (6H, m), 1.70 (2H, sextet), 0.99 (3H, t).

The first THF liquors from the above triturations were concentrated and the residue dissolved in water (200 mL) and washed with ethyl acetate (EtOAc; 250 ml). The water was concentrated to give a mixture of isomer A and B as a 1:3 mix. This was then triturated with chloroform to give an off white solid which was filtered and gave conformer B (1.29 g). This was then passed through Dowex 1×8, 50-100 mesh, ion exchange resin eluting with methanol (MeOH) to give the chloride which was triturated with Et₂O to give the chloride conformer B as an off white solid (707 mg).

¹H-NMR (CDCl₃) δ 7.86 (1H, dd), 7.21 (1H, dd), 7.04 (1H, dt), 5.74 (2H, s), 4.40 (2H, br s), 4.12-3.91 (7H, m), 3.51-3.39 (2H, m), 3.21 (2H, br s), 2.81 (3H, s), 2.66 (2H, br d), 2.56 (2H, t), 2.39-2.18 (2H, m), 2.13-1.94 (4H, m) 1.71 (2H, sextet), 0.98 (3H, t).

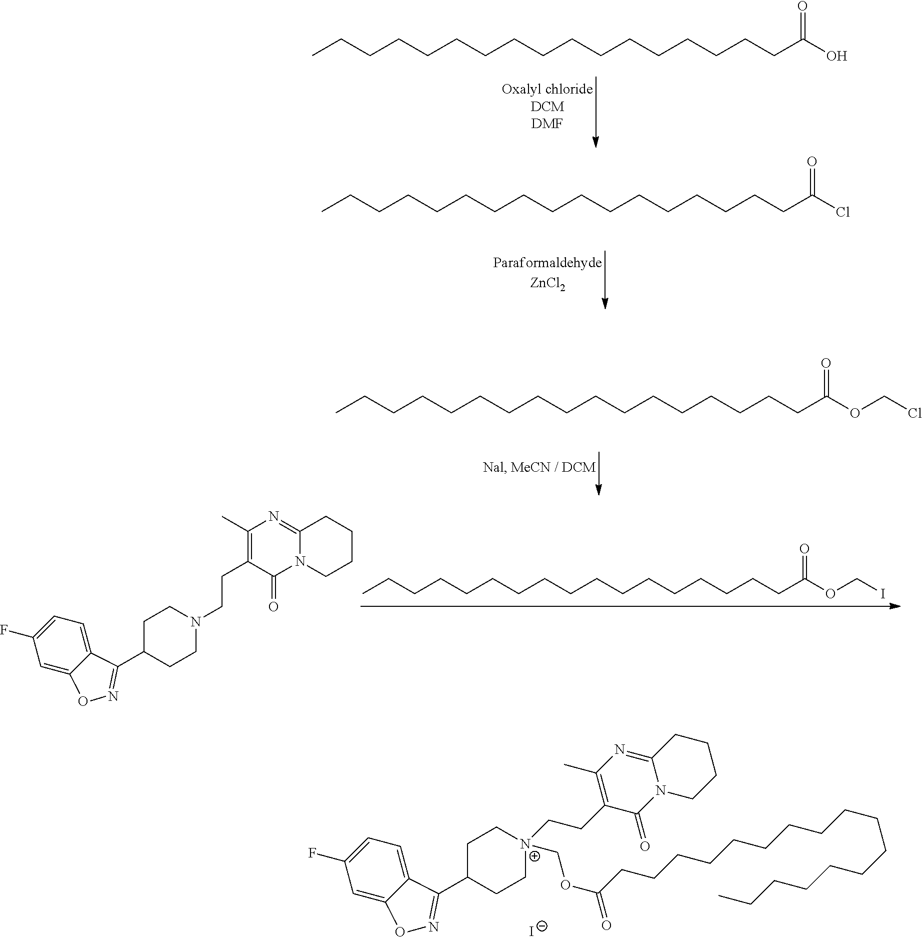

### Synthesis of Compound 44 (RSP Stearate Iodide) Step A—Formation of Acid Chloride

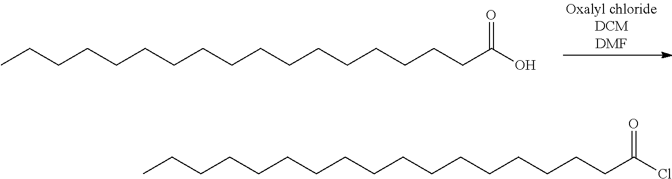

To a stirred suspension of stearic acid (20 g, 70.3 mmol) in dichloromethane (100 mL) was added oxalyl chloride (8.92 mL, 105.5 mmol). 1 drop dimethylformamide was added and the reaction stirred at 25° C. for 3 hours. The solvent was removed in vacuo and the resulting product used in the next step without further purification. ¹H-NMR (CDCl₃) δ 0.87 (3H, t), 1.20-1.40 (28H, m), 1.65-1.70 (2H, m), 2.87 (2H, t).

### Step B—Formation of Chloromethyl Alkyl Ester

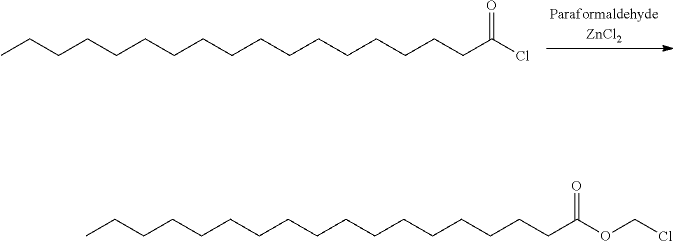

Paraformaldehyde (2.11 g, 70.3 mmol) and zinc chloride (258 mg) were added to the acid chloride prepared above and the reaction mixture was heated at 65° C. for 16 hours and then allowed to cool to 25° C. Dichloromethane (200 mL) and saturated aqueous NaHCO₃ (70 mL) were added. The aqueous emulsion was extracted with dichloromethane (2×50 mL) and the combined organic extracts washed with saturated aqueous NaHCO₃ (70 mL), brine (70 mL), and dried over MgSO₄. After filtration, the volatiles were removed and the residue purified by silica chromatography eluting with heptane to 12% dichloromethane/heptane to give a yellow solid (12.64 g, 54% yield over two steps). ¹H-NMR (CDCl₃) δ 0.86 (3H, t), 1.20-1.40 (28H, m), 1.55-1.70 (2H, m), 2.37 (2H, t), 5.70 (2H, s).

### Step C—Formation of Iodomethyl Alkyl Ester

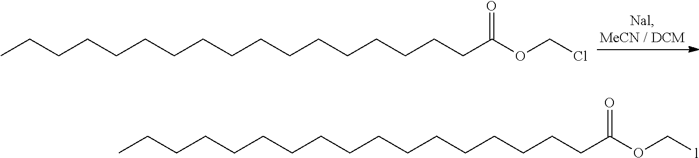

To a solution of the iodomethyl alkyl ester (12.64 g, 37.96 mmol) in acetonitrile (150 mL) and dichloromethane (75 mL) was added sodium iodide (17.07 g, 113.9 mmol). The flask was covered in tin foil to exclude light and stirred at 25° C. for 70 hours and then at 25° C. for 24 hours. The reaction mixture was partitioned between dichloromethane (200 mL) and water (150 mL). The aqueous layer was extracted with dichloromethane (2×150 mL). The combined organics were washed with saturated aqueous NaHCO₃ (200 mL), 5% aqueous sodium sulfite solution (200 mL) and brine (2×100 mL), then dried (MgSO₄) and concentrated to give the product as a yellow solid (14.53 g, 90% yield) which was not further purified. ¹H-NMR (CDCl₃) δ 0.87 (3H, t), 1.20-1.35 (28H, m), 1.55-1.70 (2H, m), 2.32 (2H, t), 5.90 (2H, s).

### Step D—Quaternisation Reaction

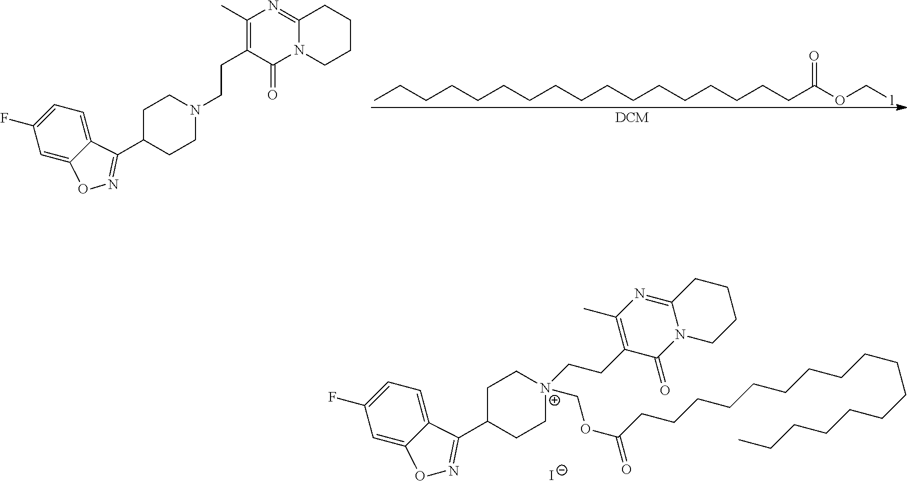

Risperidone (1.50 g, 3.65 mmol) and the iodomethyl alkyl ester (2.33 g, 5.48 mmol, 1.5 equiv) were stirred together in dichloromethane (30 mL) at 25° C. overnight. The reaction mixture was concentrated and the residue triturated with diethyl ether to give Compound 44 (2.50 g) as an approximate 1:1 mix of two conformers.

¹H-NMR (CDCl₃) δ 7.95 (1H, dd), 7.84 (1H, dd), 7.22 (2H, 2×dd), 7.11 (2H, 2×t), 5.90 (2H, s), 5.61 (2H, s), 4.80-4.60 (4H, m), 4.35-4.20 (2H, m), 4.05-3.95 (2H, m), 3.95-3.70 (8H, m), 3.65-3.55 (2H, m), 3.05-2.85 (8H, m), 2.65-2.40 (13H, m), 2.40-2.25 (5H, m), 2.00-1.85 (8H, m), 1.70-1.60 (4H, m), 1.40-1.15 (56H, m), 0.87 (6H, 2×t).

### Synthesis of Compound 39 (RSP Octanoate Chloride)

Using the general procedure described above starting from step B using octanoyl chloride. In step D, acetontirile was used instead of dichloromethane and 3 equivalents of iodomethyl octanoate was used. The iodide salt was converted to the corresponding chloride by passing through Dowex 1×8, 50-100 mesh, ion exchange resin eluting with methanol followed by an diethyl ether trituration. Compound 39 (2.017 g) was obtained as an approximate 1:1 mix of two conformers.

¹H-NMR (CDCl₃) δ 7.90 (1H, dd), 7.81 (1H, dd), 7.23 (2H, 2×dd), 7.10 (2H, 2×t), 6.01 (2H, s), 5.66 (2H, s), 4.95-4.65 (4H, m), 4.15-4.00 (4H, m), 3.95-3.80 (4H, m), 3.80-3.65 (4H, m), 3.60-3.50 (2H, m), 3.05-2.85 (8H, m), 2.65-2.40 (13H, m), 2.40-2.20 (5H, m), 2.05-1.75 (8H, m), 1.75-1.60 (4H, m), 1.40-1.20 (16H, m), 0.87 (6H, 2×t).

### Synthesis of Compound 40 (RSP Decanoate Chloride)

Synthesized using the general procedure described above starting from step B using decanoyl chloride. In step D, acetonitrile was used instead of dichloromethane and 3 equiv of iodomethyl decanoate was used. The iodide salt was converted to the corresponding chloride by passing through Dowex 1×8, 50-100 mesh, ion exchange resin eluting with methanol followed by a diethyl ether trituration to give Compound 40 (3.99 g) as an approx 1:1 mixture of 2 conformers.

¹H-NMR (CDCl₃) δ 7.91 (1H, dd), 7.81 (1H, dd), 7.23 (2H, 2×dd), 7.10 (2H, 2×t), 6.02 (2H, s), 5.67 (2H, s), 4.87 (2H, br t), 4.70 (2H, br t), 4.18-4.02 (4H, m), 3.89 (4H, dd), 3.82-3.69 (4H, m), 3.61-3.50 (2H, m), 3.08-2.87 (8H, m), 2.82-2.41 (11H, m), 2.32-2.22 (7H, m), 2.18-1.81 (8H, m), 1.73-1.58 (4H, m), 1.41-1.15 (24H, m), 0.86 (6H, 2×t).

### Synthesis of Compound 41 (RSP Laurate Iodide)

Synthesized using the general procedure described above (Example 1) starting from step B using lauroyl chloride. In step D, 3 equivalents of iodomethyl laurate was used. After diethyl ether trituration Compound 41 (3.11 g) was obtained as an approx 1:1 mixture of 2 conformers.

¹H-NMR (CDCl₃) δ 7.97 (1H, dd), 7.83 (1H, dd), 7.24 (2H, 2×dd), 7.11 (2H, 2×t), 5.89 (2H, s), 5.61 (2H, s), 4.72-4.58 (4H, m), 4.32-4.17 (2H, m), 4.06 (2H, br t), 3.92-3.72 (8H, m), 3.64-3.56 (2H, m), 3.06-2.87 (8H, m), 2.68-2.52 (12H, m), 2.39-2.28 (6H, m), 2.02-1.89 (8H, m), 1.68-1.61 (4H, m), 1.39-1.18 (32H, m), 0.87 (6H, 2×t).

### Synthesis of Compound 42 (RSP Myristate Iodide)

Synthesized using the general procedure described above starting from step B using myristoyl chloride. In step D, 3 equivalents of iodomethyl myristate was used. Compound 42 (3.23 g) was obtained as an approximate 1:1 mix of two conformers.

¹H-NMR (CDCl₃) δ 7.95 (1H, dd), 7.84 (1H, dd), 7.22 (2H, 2×dd), 7.11 (2H, 2×t), 5.89 (2H, s), 5.60 (2H, s), 4.80-4.60 (4H, m), 4.30-4.15 (2H, m), 4.05-3.95 (2H, m), 3.95-3.70 (8H, m), 3.60-3.55 (2H, m), 3.05-2.85 (8H, m), 2.65-2.40 (13H, m), 2.40-2.25 (5H, m), 2.00-1.85 (8H, m), 1.75-1.60 (4H, m), 1.40-1.15 (40H, m), 0.86 (6H, 2×t).

### Synthesis of Compound 43 (RSP Palmitate Iodide)

Synthesized using the general procedure described above starting from step B using palmitoyl chloride. In step D, 3 equiv of iodomethyl palmitate was used. After diethyl ether trituration Compound 43 (4.13 g) was obtained as an approx 1:1 mixture of 2 conformers.

¹H-NMR (CDCl₃) δ 7.94 (1H, dd), 7.84 (1H, dd), 7.24 (2H, 2×dd), 7.11 (2H, 2×t), 5.89 (2H, s), 5.60 (2H, s), 4.77-4.63 (4H, m), 4.31-4.18 (2H, m), 4.05-4.02 (2H, m), 3.89 (4H, t), 3.78 (4H, br t), 3.62-3.57 (2H, m), 3.06-2.87 (8H, m), 2.64-2.48 (12H, m), 2.39-2.27 (6H, m), 1.99-1.88 (8H, m), 1.64-1.59 (4H, m), 1.39-1.18 (48H, m), 0.87 (6H, 2×t).

### Synthesis of Compound 46 (RSP Pivalate Chloride)

Synthesized using the general procedure described above starting from step C using chloromethyl pivalate. In step D, acetontirile was used instead of dichloromethane and 3 equivalents of iodomethyl pivalate was used. The iodide salt was converted to the corresponding chloride by passing through Dowex 1×8, 50-100 mesh, ion exchange resin eluting with methanol followed by a diethyl ether/tetrahydrofuran trituration to give Compound 46 (2.91 g) as an approx 1:1 mixture of 2 conformers.

¹H-NMR (d⁶-MeOH) δ 7.99 (1H, dd), 7.91 (1H, dd), 7.45 (2H, 2×dd), 7.22 (2H, 2×t), 5.62 (2H, s), 5.55 (2H, s), 3.98-3.82 (8H, m), 3.78-3.52 (10H, m), 3.12-2.89 (8H, m), 2.62-2.33 (14H, m), 2.05-1.84 (8H, m), 1.35 (9H, s), 1.32 (9H, s).

### Synthesis of Compound 47 (RSP Dimethylbutyrate Iodide)

Synthesized using the general procedure described above starting from step B using 2,2-dimethylbutyryl chloride. In step D, 3 equivalents of iodomethyl 2,2-dimethylbutyrate was used. Compound 47 (3.14 g) was obtained as an approximate 1:1 mix of two conformers.

¹H-NMR (CDCl₃) δ 7.95 (1H, dd), 7.84 (1H, dd), 7.23 (2H, 2×dd), 7.11 (2H, 2×t), 5.92 (2H, s), 5.64 (2H, s), 4.80-4.55 (4H, m), 4.30-4.15 (2H, m), 4.10-3.95 (2H, m), 3.95-3.65 (8H, m), 3.65-3.55 (2H, m), 3.10-2.85 (8H, m), 2.75-2.45 (9H, m), 2.40-2.25 (5H, m), 2.05-1.85 (8H, m), 1.75-1.55 (4H, m), 1.30-1.20 (12H, m), 0.90 (6H, 2×t).

### Synthesis of Compound 162 (RSP 2-Methyl Cyclohexyl Carboxylate Iodide)

Made using the general procedure described in Example 1, starting from 1-methyl cyclohexane carboxylic acid. After diethyl ether trituration compound 162 (2.66 g) was obtained as an approx 1:1 mixture of 2 conformers.

¹H-NMR (300 MHz, CDCl₃) δ 7.94 (1H, dd), 7.83 (1H, dd), 7.25-7.22 (2H, m), 7.14-7.08 (2H, m), 5.93 (2H, s), 5.65 (2H, s), 4.79-4.54 (4H, m), 4.24-3.53 (16H, m), 3.11-2.89 (8H, m), 2.72-2.53 (8H, m), 2.41-2.27 (4H, m), 2.14-1.89 (12H, m), 1.69-1.27 (22H, m).

### Synthesis of Compound 163 (RSP Isobutyrate Iodide)

Made using the general procedure starting from isobutyryl chloride. After dissolving in a minimum amount of tetrahydrofuran followed by precipitation with diethyl ether compound 163 (2.23 g) was obtained as an approx 1:1 mixture of 2 conformers.

¹H-NMR (300 MHz, CDCl₃) δ 7.93 (1H, dd), 7.83 (1H, dd), 7.25-7.22 (2H, m), 7.14-7.08 (2H, m), 5.90 (2H, s), 5.63 (2H, s), 4.75 (2H, br t), 4.65 (2H, br t), 4.33-4.19 (2H, m), 4.07-4.02 (2H, m), 3.89 (4H, dt), 3.82-3.71 (4H, m), 3.62-3.57 (2H, m), 3.07-3.02 (2H, m), 2.98-2.79 (8H, m), 2.68-2.63 (2H, m), 2.53-2.41 (6H, m), 2.39-2.28 (5H, m), 2.03-1.88 (8H, m), 1.27 (12H, 2×d).

### Synthesis of Compound 49 (RSP Dimethyl Myristate Iodide)

Synthesis of methyl 2,2-dimethyltetradecanoate

To a stirred solution of diisopropylamine (6.90 mL, 49.0 mmol) in tetrahydrofuran (50 mL) under Ar (g) at −7° C. was added n-butyl lithium (2.3M in hexanes, 21.3 mL, 49.0 mmol) dropwise via a dropping funnel keeping the temperature between 0° C. and 5° C. The reaction was stirred at −7° C. for 30 min and then cooled to −78° C. Methyl isobutyrate (5.61 mL, 49.0 mmol) was added and the reaction stirred at −78° C. for 1.5 hours. 1-Iodododecane (13.05 g, 44.1 mmol) in tetrahydrofuran (10 mL) was added dropwise via a dropping funnel keeping the temperature below −70° C. Further tetrahydrofuran (40 mL) was added over 5 minutes to aid stirring. After complete addition the reaction was stirred at −78° C. for approx. 2 hours and then allowed to slowly warm to room temperature overnight.

The reaction was quenched with saturated aqueous NH₄Cl (100 mL) and diluted with ethyl acetate (100 mL). The aqueous layer was extracted with ethyl acetate (2×50 mL) and the combined organics washed with brine (50 mL) and dried over MgSO₄. After filtration, the volatiles were removed. The reaction was repeated in a similar manner using methyl isobutyrate (15.05 mL, 31.27 mmol). The two crude batches were combined and purified by silica chromatography eluting heptane to 50% dichloromethane/heptane to give methyl 2,2-dimethyl myristate (31.7 g).

Synthesis of 2,2-dimethyltetradecanoic acid

To a stirred solution of methyl 2,2-dimethyltetradecanoate (31.7 g, 117.2 mmol) in ethanol (234 mL) was added 2M NaOH (117 mL, 234.4 mmol). The reaction was stirred at 25° C. overnight. NaOH (4.69 g, 117 mmol) was added and the reaction heated at 50° C. for 24 hours. NaOH (4.69 g, 117 mmol) was added and the reaction heated to 100° C. for 4 hours and then cooled to 25° C. 4M HCl (140 mL) was added to acidify. Ethyl acetate (200 mL) was added and the layers separated. The aqueous was extracted with ethyl acetate (2×100 mL) and the combined organics concentrated in vacuo. The residue was partitioned between ethyl acetate (200 mL) and brine (100 mL). The organic layer was washed with brine (50 mL) and dried over MgSO₄. After filtration, the volatiles were removed to give 2,2-dimethyltetradecanoic acid (26.9 g).

Compound 49 was made using the general procedure starting from 2,2-dimethyltetradecanoic acid (synthesized as described above). After diethyl ether trituration compound 49 (1.91 g) was obtained as an approximately 1:1 mixture of 2 conformers.

¹H-NMR (300 MHz, CDCl₃) δ 7.94 (1H, dd), 7.84 (1H, dd), 7.24 (2H, 2×dd), 7.11 (2H, 2×t), 5.90 (2H, s), 5.62 (2H, s), 4.83-4.58 (4H, m), 4.36-4.19 (2H, m), 4.09-3.97 (2H, m), 3.97-3.65 (8H, m), 3.65-3.52 (2H, m), 3.12-2.83 (8H, m), 2.73-2.44 (9H, m), 2.44-2.23 (5H, m), 2.04-1.83 (8H, m), 1.67-1.52 (4H, m), 1.36-1.13 (52H, m), 0.87 (6H, 2×t).

Synthesis of Compound 164 (RSP 2-propyl pentanoate iodide)

Made using the general procedure starting from 2,2-di-n-propylacetic acid. After diethyl ether trituration compound 164 (2.75 g) was obtained as an approximately 1:1 mixture of 2 conformers.

¹H-NMR (300 MHz, CDCl₃) δ 7.94 (1H, dd), 7.85 (1H, dd), 7.24 (2H, 2×dd), 7.11 (2H, 2×t), 5.92 (2H, s), 5.64 (2H, s), 4.78-4.57 (4H, m), 4.33-4.19 (2H, m), 4.07-3.97 (2H, m), 3.95-3.66 (8H, m), 3.66-3.55 (2H, m), 3.11-2.84 (8H, m), 2.71-2.44 (11H, m), 2.44-2.25 (5H, m), 2.04-1.83 (8H, m), 1.74-1.45 (8H, m), 1.40-1.23 (8H, m), 0.91 (12H, m).

### Synthesis of Compound 165 (RSP Dimethylpentanoate Iodide)

Made using the general procedure starting from 2,2-dimethylvaleric acid. After diethyl ether trituration compound 165 (2.50 g) was obtained as an approximately 1:1 mixture of 2 conformers.

¹H-NMR (300 MHz, CDCl₃) δ 7.93 (1H, dd), 7.83 (1H, dd), 7.27-7.20 (2H, m), 7.15-7.07 (2H, m), 5.90 (2H, s), 5.62 (2H, s), 4.80-4.62 (4H, m), 4.33-4.20 (2H, m), 4.08-4.00 (2H, m), 3.93-3.85 (4H, m), 3.81-3.65 (4H, m), 3.62-3.54 (2H, m), 3.08-2.85 (8H, m), 2.70-2.45 (9H, m), 2.39-2.27 (5H, m), 2.02-1.84 (8H, m), 1.62-1.52 (4H, m), 1.32-1.22 (16H, m), 0.91 (6H, 2×t).

### Synthesis of Compound 166 (RSP Dimethyl Hexanoate Iodide)

Made in a similar manner to compound 49 from methyl isobutyrate and 1-iodobutane. After diethyl ether trituration compound 166 (2.75 g) was obtained as an approximately 1:1 mixture of 2 conformers.

¹H-NMR (300 MHz, CDCl₃) δ 7.94 (1H, dd), 7.84 (1H, dd), 7.28-7.21 (2H, m), 7.16-7.06 (2H, m), 5.91 (2H, s), 5.62 (2H, s), 4.82-4.59 (4H, m), 4.34-4.18 (2H, m), 4.09-3.97 (2H, m), 3.95-3.64 (8H, m), 3.64-3.53 (2H, m), 3.10-2.84 (8H, m), 2.72-2.45 (9H, m), 2.43-2.26 (5H, m), 2.04-1.83 (8H, m), 1.65-1.53 (4H, m), 1.37-1.12 (20H, m), 0.88 (6H, 2×t).

### Example 2

### Paliperidone

### Preparation of Paliperidone Methylthiomethyl Ether (PPD-MTM)

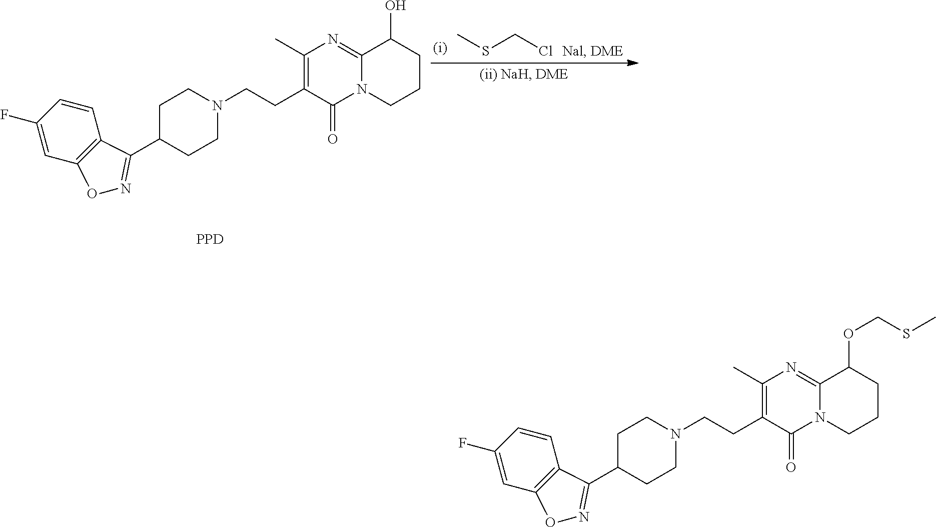

To a stirred suspension of sodium iodide (7.03 g, 46.9 mmol) in 1,2-dimethoxyethane (100 mL) was added chloromethyl methyl sulfide. The reaction was stirred for 1.5 hours.

Meanwhile paliperidone (10 g, 23.45 mmol) was suspended in 1,2-dimethoxyethane (300 mL) under argon and heated to improve solubility. The mixture was then allowed to cool to 25° C. The alkylating agent prepared above was added to this mixture followed by sodium hydride portionwise over approximately 10 mins under argon. This procedure was repeated simultaneously using another 10 g paliperidone.

After approximately 1.5 hours both batches were combined by carefully pouring into water (1 L) and the aqueous was extracted with ethyl acetate (3×300 mL). The combined organic extracts were washed with saturated NaHCO₃ solution, brine and dried over MgSO₄. After filtration, the volatiles were removed and the residue purified by silica chromatography eluting with ethyl acetate/dichloromethane/methanol (1:1:0.1 to 1:1:0.17) to give the title compound (14.79 g, 64% yield).

¹H NMR (CDCl₃, 300 MHz) δ 1.9-2.5 (m, 16H), 2.6-2.9 (m, 4H), 3.1-3.3 (m, 3H), 3.75-3.9 (m, 1H), 4.00-4.05 (m, 1H), 4.7 (t, 1H), 4.8 (d, 1H), 5.0 (d, 1H), 7.0-7.1 (m, 1H), 7.2-7.25 (m, 1H), 7.7-7.8 (m 1H); m/z (M⁺H) 487.3.

### Synthesis of Compound 156—PPD Decanoate

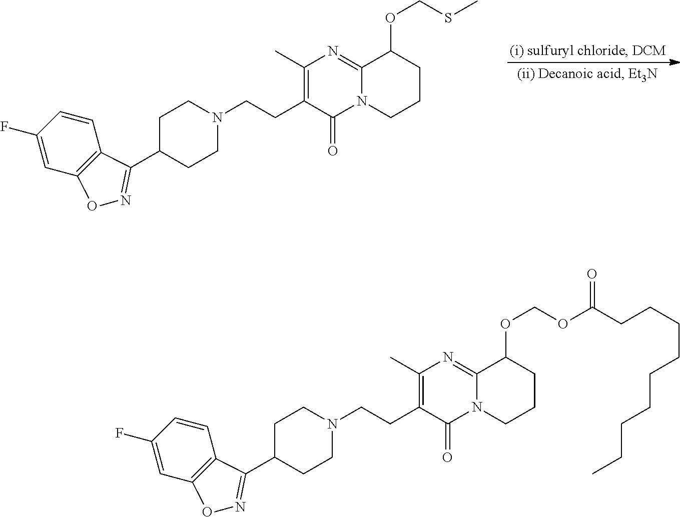

To a stirred suspension of PPD-MTM (1.63 g, 3.35 mmol) in dichloromethane (16 mL) under argon at 78° C. was added 2M sulfuryl chloride in dichloromethane (1.84 mL, 3.69 mmol) over 10 minutes in 0.05 mL portions. After 30 min decanoic acid (2.31 g, 13.40 mmol) was added in one portion followed by triethylamine (1.87 mL, 13.40 mmol) over 5 minutes in 0.05 mL portions at 78° C. After 1 hour at 78° C. the reaction was allowed to warm to 25° C. and then stirred for 70 minutes. The reaction mixture was poured into dichloromethane (20 mL) and saturated NaHCO₃ solution (20 mL). The aqueous phase was extracted with dichloromethane (2×10 mL) and the combined organic extracts were dried over MgSO₄. After filtration, the volatiles were removed and the residue purified by silica chromatography eluting with ethyl acetate/dichloromethane/methanol (1:1:0.17) to give the title compound (0.82 g, 40% yield).

¹H NMR (CDCl₃, 300 MHz) δ 0.85 (t, 3H), 1.15-1.4 (m, 12H), 1.55-1.7 (m, 2H), 1.85-2.0 (m, 2H), 2.0-2.2 (m, 6H), 2.2-2.5 (m, 7H), 2.5-2.9 (m, 4H), 3.0-3.3 (m, 3H), 3.8-3.9 (m, 1H), 3.95-4.05 (m, 1H), 4.65 (t, 1H), 5.45 (d, 1H), 5.5 (d, 1H), 7.0-7.1 (m, 1H), 7.2 (m, 1H), 7.65-7.80 (m, 1H); m/z (M⁺H) 611.5.

### Synthesis of Compound 154—PPD Butyrate

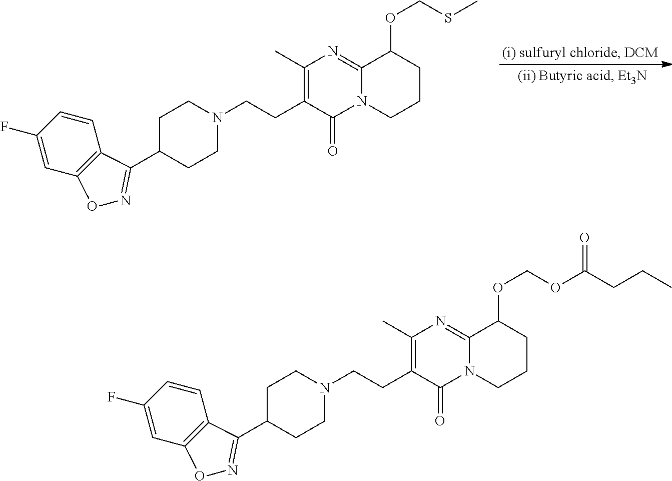

Prepared in a similar manner to compound 156 using PPD-MTM (2.3 g, 4.73 mmol) to give compound 166 (0.810 g, 32% yield).

¹H NMR (CDCl₃, 300 MHz) δ 0.95 (t, 3H), 1.6-1.7 (m, 2H), 1.85-2.0 (m, 2H), 2.0-2.2 (m, 6H), 2.2-2.35 (m, 7H), 2.5-2.7 (m, 2H), 2.7-2.9 (m, 2H), 3.0-3.3 (m, 3H), 3.8-3.9 (m, 1H), 3.95-4.05 (m, 1H), 4.65 (t, 1H), 5.45 (d, 1H), 5.5 (d, 1H), 7.0-7.1 (m, 1H), 7.2 (m, 1H), 7.65-7.8 (m, 1H); m/z (M⁺H) 527.2.

### Synthesis of Compound 152-PPD Acetate

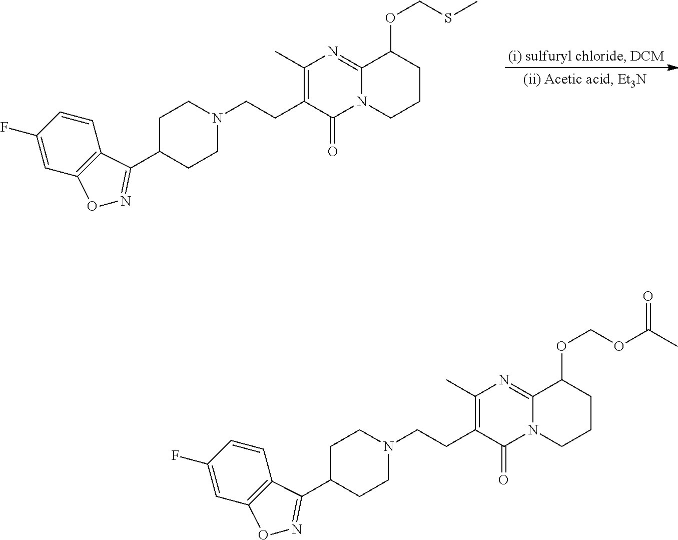

Prepared in a similar manner to compound 156 using PPD-MTM ether (2.2 g, 4.52 mmol) to give Compound 152 (0.804 g, 35% yield).

¹H NMR (CDCl₃, 300 MHz) δ 1.7-2.0 (m, 2H), 2.0-2.25 (m, 9H), 2.25-2.45 (m, 5H), 2.5-2.7 (m, 2H), 2.7-2.9 (m, 2H), 3.0-3.3 (m, 3H), 3.8-3.9 (m, 1H), 4.0-4.1 (m, 1H), 4.65 (t, 1H), 5.45 (d, 1H), 5.5 (d, 1H), 7.0-7.1 (m, 1H), 7.2 (m, 1H), 7.65-7.8 (m, 1H); m/z (M⁺H) 499.1.

### Synthesis of Compound 155—PPD Hexanoate

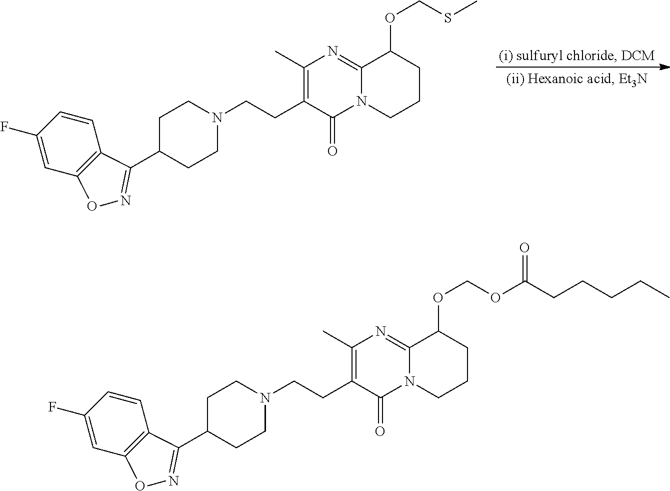

Prepared in a similar manner to compound 156 using PPD-MTM ether (2.5 g, 5.14 mmol) to give Compound 155 (0.892 g, 31% yield).

¹H NMR (CDCl₃, 300 MHz) δ 0.8-1.0 (t, 3H), 1.2-1.4 (m, 4H), 1.55-1.8 (m, 2H), 1.8-2.0 (m, 2H), 2.0-2.2 (m, 6H), 2.2-2.4 (m, 7H), 2.45-2.65 (m, 2H), 2.7-2.9 (m, 2H), 3.0-3.3 (m, 3H), 3.75-3.95 (m, 1H), 3.95-4.1 (m, 1H), 4.65 (t, 1H), 5.45 (d, 1H), 5.5 (d, 1H), 7.0-7.1 (m, 1H), 7.2 (m, 1H), 7.65-7.8 (m, 1H); m/z (M⁺H) 555.2.

### Synthesis of Compound 157—PPD Palmitate

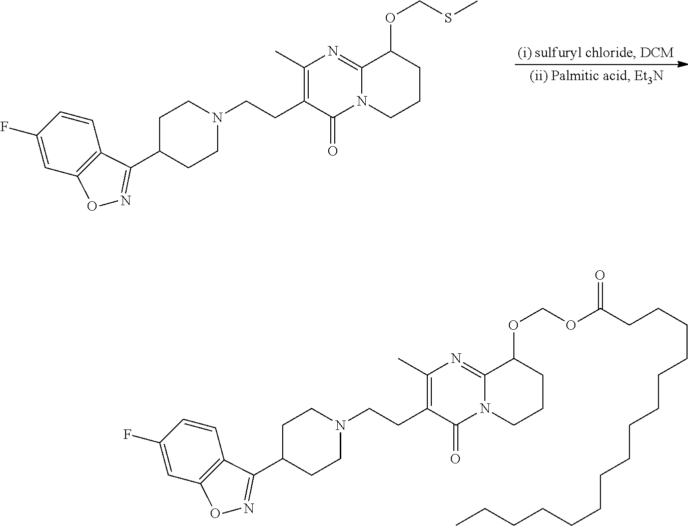

Prepared in a similar manner to compound 156 using PPD-MTM ether (2.5 g, 5.14 mmol) to give Compound 157 (1.25 g, 35% yield).

¹H NMR (CDCl₃, 300 MHz) δ 0.8-0.9 (t, 3H), 1.15-1.35 (m, 24H), 1.55-1.65 (m, 2H), 1.85-2.0 (m, 2H), 2.0-2.2 (m, 6H), 2.25-2.4 (m, 7H), 2.5-2.65 (m, 2H), 2.7-2.85 (m, 2H), 3.0-3.3 (m, 3H), 3.75-3.9 (m, 1H), 3.95-4.05 (m, 1H), 4.65 (t, 1H), 5.45 (d, 1H), 5.5 (d, 1H), 7.0-7.1 (m, 1H), 7.2 (m, 1H), 7.65-7.75 (m, 1H); m/z (M⁺H) 695.8.

### Synthesis of Compound 153—PPD Valerate

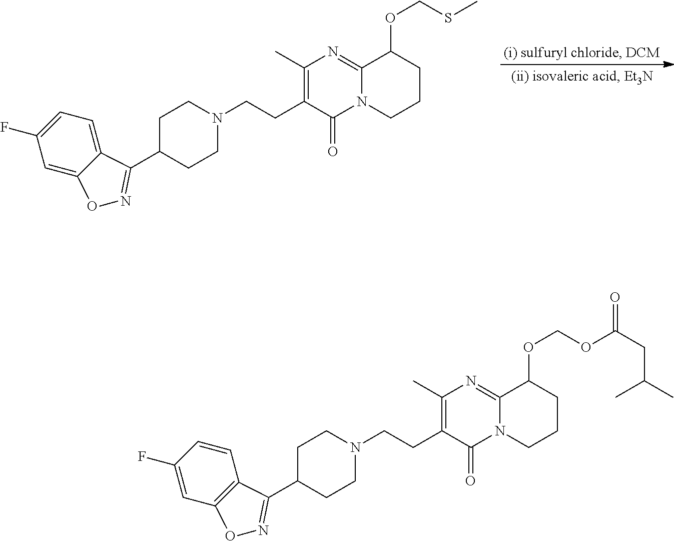

Prepared in a similar manner to compound 156 using PPD-MTM ether (2.5 g, 5.14 mmol) to give Compound 153 (0.954 g, 34% yield).

¹H NMR (CDCl₃, 300 MHz) δ 0.95 (d, 6H), 1.7-2.0 (m, 3H), 2.0-2.45 (m, 13H), 2.5-2.65 (m, 2H), 2.7-2.9 (m, 2H), 3.0-3.3 (m, 3H), 3.75-3.9 (m, 1H), 3.95-4.05 (m, 1H), 4.65 (t, 1H), 5.45 (d, 1H), 5.5 (d, 1H), 7.0-7.1 (m, 1H), 7.2 (m, 1H), 7.65-7.75 (m, 1H); m/z (M⁺H) 541.2.

### Preparation of Chloromethyl Dibenzylcarbamate

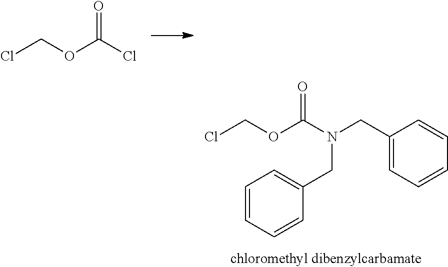

To a stirred solution of chloromethyl chloroformate (2 g, 15.5 μmol) in dichloromethane (30 mL) at 0° C. under argon was added dibenzylamine (2.98 mL, 15.51 mmol) followed by diisopropylethylamine (4.05 mL, 23.3 mmol). The solution was stirred at 0° C. under argon for 30 mins and then allowed to warm to 25° C. After stirring for a further 1 h 45 mins the reaction mixture was diluted with saturated NaHCO₃ solution (30 mL). The organic phase was washed with saturated NaHCO₃ solution (30 mL), 1M HCl solution (2×30 mL), brine (30 mL) and dried over MgSO₄. After filtration, the volatiles were removed to give the title compound (4.19 g, 93% yield).

¹H NMR (CDCl₃, 300 MHz) δ 4.4-4.5 (m, 4H), 5.9 (s, 2H), 7.15-7.45 (m, 10H).

### Preparation of Iodomethyl Dibenzylcarbamate

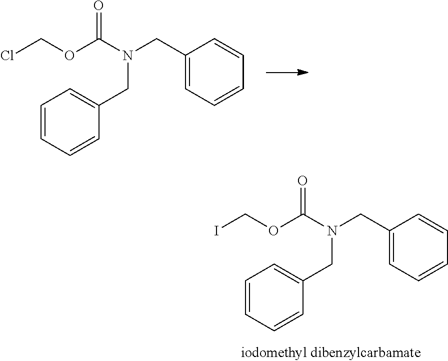

To a stirred solution of chloromethyl dibenzylcarbamate (1.8 g, 6.21 mmol) in acetonitrile (18 mL) was added sodium iodide (2.79 g, 18.64 mmol). The flask was covered with tin foil to exclude light and the reaction stirred at 25° C. for 18 hours. The mixture was partitioned between dichloromethane (50 mL) and water (50 mL). The aqueous was extracted with dichloromethane (2×50 mL) and the combined organic extracts washed with 5% aqueous sodium sulfite (50 mL), saturated NaHCO₃ solution (50 mL), brine (50 mL) and dried over MgSO₄. After filtration, the volatiles were removed to give the title compound (1.95 g, 82% yield).

¹H NMR (CDCl₃, 300 MHz) δ 4.3-4.5 (m, 4H), 6.1 (s, 2H), 7.1-7.55 (m, 10H).

### Synthesis of Compound 158—PPD Dibenzyl Carbamate

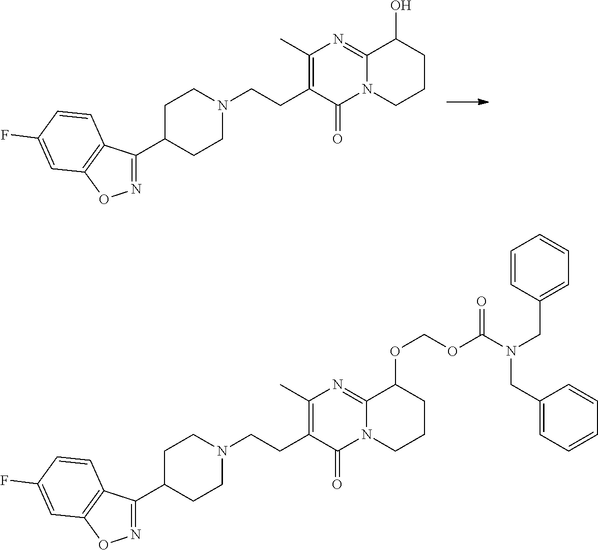

To a stirred solution of paliperidone (700 mg, 1.64 mmol) in tetrahydrofuran (25 mL) under Ar(g) was added 60% sodium hydride in oil (98.5 mg, 2.46 mmol) in one portion. After 20 mins at 25° C. the reaction was cooled to 0° C. After 5 mins iodomethyl dibenzylcarbamate (625.7 mg, 1.64 mmol) was added in one portion followed by tetrahydrofuran (2.5 mL). The reaction was stirred at 0° C. for 3 hours 45 min. and then quenched by slow addition of water (2 mL). After warming to 25° C. the mixture was poured into water (20 mL) and extracted with ethyl acetate (3×50 mL). Brine (20 mL) was added to aid layer separation. The combined organic extracts were washed with brine (20 mL) and dried over MgSO₄. After filtration, the volatiles were removed and the residue purified by silica chromatography eluting with ethyl acetate/dichloromethane/methanol (1:1:0.2) to give the title compound (647 mg, 58% yield).

¹H NMR (CDCl₃, 300 MHz) δ 1.75-1.95 (m, 2H), 2.0-2.4 (m, 11H), 2.4-2.6 (m, 2H), 2.7-2.8 (m, 2H), 3.0-3.25 (m, 3H), 3.75-3.9 (m, 1H), 3.95-4.05 (m, 1H), 4.3-4.65 (m, 5H), 5.55 (d, 1H), 5.65 (d, 1H), 7.0-7.1 (m, 1H), 7.1-7.4 (m, 11H), 7.65-7.75 (m, 1H); m/z (M⁺H) 680.5.

### Example 3

### Pharmacokinetic Evaluation of Paliperidone Prodrugs in Rats

Two PK studies were conducted using intramuscular (IM) administration in rats of water-insoluble paliperidone prodrugs and the results were combined in The FIGURE.

Study 1

Animals: 18 Male Sprague-Dawley rats (Charles River Laboratories, Wilmington, Mass.) were used in the study. Three groups of 6 rats were used and are referred to in this study as Groups A, B and C. Rats were approximately 350-375 g at time of arrival. Rats are housed 2 per cage with ad libitum chow and water. Environmental conditions in the housing room: 64-67° F., 30% to 70% relative humidity, and 12:12-h light:dark cycle. All experiments were approved by the institutional animal care and use committee.

Test Compounds: The following formulations of paliperidone prodrug compounds of the invention were used in the study.

| Study | | Dose | | |
|---|---|---|---|---|
| Group | Test Cpd | mg | route | Dosing Vehicle |
| A | Paliperidone-O- | 22.8 | IM | Milled crystalline |
| | methyleneoxy- | | | suspension in 1% |
| | butyrate | | | HPMC in PBS |
| | (Compound 154) | | | saline with 0.2% |
| | | | | Tween 20 |
| B | Paliperidone-O- | 13.6 | IM | Milled crystalline |
| | methyleneoxy- | | | suspension in 1% |
| | hexanoate | | | HPMC in PBS |
| | (Compound 155) | | | saline with 0.2% |
| | | | | Tween 20 |
| C | Paliperidone-O- | 20 | IM | Milled crystalline |
| | methyleneoxy- | | | suspension in 1% |
| | palmitate | | | HPMC in PBS |
| | (Compound 157) | | | saline with 0.2% |
| | | | | Tween 20 |

Pharmacokinetics study: Rats were dosed IM by means of a 23 gauge, 1 inch needle with 1 cc syringe 0.3 mL suspension was withdrawn from the vial containing the test compound in suspension. The rat was injected in the muscles of the hind limb after anesthesia with isoflurane. Blood samples were collected via a lateral tail vein after brief anesthesia with Isoflurane. A 27½ G needle and 1 cc syringe without an anticoagulant was used for the blood collection. Approximately 35 0 μL of whole blood was collected at each sampling time point of 6 hours, 24 hours and 2, 5, 7, 9, 12, 14, 21, 28, 35 days after administration. Once collected, whole blood was immediately transferred to tubes containing K2 EDTA, inverted 10-15 times and immediately placed on ice. The tubes were centrifuged for 2 minutes at >14,000 g's (11500 RPMs using Eppendorf Centrifuge 5417C, F45-30-11 rotor) at room temperature to separate plasma. Plasma samples were transferred to labeled plain tubes (MICROTAINER®) and stored frozen at <−70° C.

Data Analysis: Drug concentrations in plasma samples were analyzed by liquid chromatography-mass spectroscopy (LC-MS/MS) using appropriate parameters for each compound. Half-life of Paliperidone, maximal concentration (Cmax), time to maximal concentration (Tmax), and AUC were calculated by using WinNonlin version 5.2 software (Pharsight, St. Louis, Mo.).

Results: The results of Study 1 were combined with Study 2 and are shown in The FIGURE as discussed below.

Study 2

Animals: 18 Male Sprague-Dawley rats (Charles River Laboratories, Wilmington, Mass.) were used in the study. Three groups of 6 rats were used and are referred to in this study as Groups A, B and C. Rats were approximately 350-375 g at time of arrival. Rats are housed 2 per cage with ad libitum chow and water. Environmental conditions in the housing room: 64-67° F., 30% to 70% relative humidity, and 12:12-h light:dark cycle. All experiments were approved by the institutional animal care and use committee.

Test Compounds: The following formulations of paliperidone prodrug compounds of the invention were used in the study.

| Study | | Dose | | |
|---|---|---|---|---|
| Group | Test Cpd | mg/kg | route | Dosing Vehicle |
| A | Paliperidone-O- | 67 | IM | Milled crystalline |
| | methyleneoxy- | | | suspension in 1% |
| | palmitate | | | HPMC in PBS |
| | (Compound 157) | | | saline with 0.2% |
| | | | | Tween 20 |
| B | Paliperidone | 67 | IM | Milled crystalline |
| | palmitate (PP; no | | | suspension in 1% |
| | formaldehyde | | | HPMC in PBS |
| | linker) | | | saline with 0.2% |
| | | | | Tween 20 |
| C | Paliperidone-O- | 67 | IM | Milled crystalline |
| | methyleneoxy- | | | suspension in 1% |
| | decanoate | | | HPMC in PBS |
| | (Compound 156) | | | saline with 0.2% |
| | | | | Tween 20 |

The paliperidone palmitate compound without the formaldehyde linker is the known Janssen compound Paliperidone Palmitate (PP; same active ingredient as in INVEGA® SUSTENNA®) having the formula:

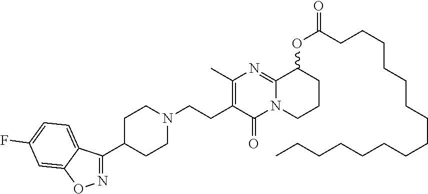

Pharmacokinetics study: Rats were dosed IM by means of a 23 gauge, 1 in. needle with 1 cc syringe 0.3 mL suspension was withdrawn from the vial containing the test compound in suspension. The rat was injected in the muscles of the hind limb after anesthesia with isoflurane. Blood samples were collected via a lateral tail vein after brief anesthesia with Isoflurane. A 27½ G needle and 1 cc syringe without an anticoagulant was used for the blood collection. Approximately 350 μL of whole blood was collected at each sampling time point of 6 hours, 24 hours and 2, 5, 7, 9, 12, 14, 21, 28, 35 days after administration. Once collected, whole blood was immediately transferred to tubes containing K2 EDTA, inverted 10-15 times and immediately placed on ice. The tubes were centrifuged for 2 minutes at >14,000 g's (11500 RPMs using Eppendorf Centrifuge 5417C, F45-30-11 rotor) at room temperature to separate plasma. Plasma samples were transferred to labeled plain tubes (MICROTAINER®) and stored frozen at <−70° C.

Data Analysis: Drug concentrations in plasma samples were analyzed by liquid chromatography-mass spectroscopy (LC-MS/MS) using appropriate parameters for each compound. Half-life, maximal concentration, time to maximal concentration and AUC were calculated by using WinNonlin version 5.2 software (Pharsight, St. Louis, Mo.).

Results: The results of Study 1 were combined with Study 2 and data are shown in The FIGURE. Data for the paliperidone-O-methyleneoxy-palmitate prodrug (Compound 157) was consistent and reproducible between Study 1 and Study 2. For clarity and illustration, PK data from Study 2 was included in the graph shown in The FIGURE. As shown in The FIGURE, the Cₘₐₓ for both of the palmitate prodrug compounds (Compound 157 having the methyleneoxy-linker and the reference compound PP having no linker) was lower than that of the other compounds. The Tₘₐₓ was also delayed for both the palmitate compounds.

### Example 4

### Pharmacodynamic Studies Using an Amphetamine-Induced Locomotion Model

Introduction: Prodrugs of the invention useful in the treatment of schizophrenia and bipolar disorder are expected to show predictive validity in rodent models of hyperlocomotion. D-Amphetamine-induced locomotion is postulated to mimic the dopaminergic hyperactivity which forms the basis for the “dopamine hypothesis” of schizophrenia. The AMPH-induced hyperactivity model provides a simple, initial screen of antipsychotic compound efficacy. See, Fell et al., Journal of Pharmacology and Experimental Therapeutics (2008) 326:209-217. Amphetamine induced hyperactivity is used to screen various doses of prodrug formulations of paliperidone, risperidone, iloperidone, lurasidone, perospirone, and ziprasidone to measure pharmacodynamic efficacy in an acute hyperlocomotion paradigm. The hypothesis of the study is that PO administration of paliperidone, risperidone, iloperidone, lurasidone, perospirone, and ziprasidone prodrug formulations, which result in efficacious plasma concentrations will produce a significant attenuation of AMPH-induced locomotion.

General behavior and activity can be measured in experimental animals (typically rats and mice) in order to assess psychomotor stimulant properties, anxiogenic/anxiolytic or sedative properties of a drug. As such, open-field studies can provide insight into the behavioral effects of test compounds. Certain prodrugs of the present invention are useful in the treatment of schizophrenia and bipolar disorder. Paliperidone, risperidone, iloperidone, lurasidone, perospirone, and ziprasidone are parent drugs from which prodrugs of the invention are derived that are useful in the treatment of schizophrenia and bipolar disorder. Such paliperidone, risperidone, iloperidone, lurasidone, perospirone, and ziprasidone prodrugs of the invention show predictive validity in rodent models of hyperlocomotion. D-Amphetamine-induced locomotion is postulated to mimic the dopaminergic hyperactivity which forms the basis for the “dopamine hypothesis” of schizophrenia. Likewise, glutamate NMDA receptor antagonist (MK-801, PCP, etc.) induced locomotion is postulated to mimic the NMDA hypoactivity hypothesis of schizophrenia (Fell et al., supra). These tests of drug-induced hyperactivity provide simple, initial screens of antipsychotic compound efficacy. Amphetamine induced hyperactivity will be used to screen various prodrugs of Page 110 of 126 paliperidone, risperidone, iloperidone, lurasidone, perospirone, and ziprasidone, administered PO in oil solutions, to measure pharmacodynamic efficacy. The results of the D-AMPH induced locomotion done in this study will be compared to the historical results of subcutaneous (S.C.) paliperidone, risperidone, iloperidone, lurasidone, perospirone, and ziprasidone administration on D-AMPH. The hypothesis of the study is that PO exposure to paliperidone, risperidone, iloperidone, lurasidone, perospirone, and ziprasidone prodrugs will display efficacy in in vivo measures of antipsychotic efficacy.

Materials: Experimental animals: 12, Sprague Dawley rats are purchased from Charles River Laboratory. The rats are approximately 90 days old, and weighed in the range of 350-275 grams upon receipt from the supplier. One rat is placed in each cage and allowed to acclimate for about 1 week. The rats are provided with food and water ad libitum.

Dosing solution of D-Amphetamine (D-AMPH): D-AMPH is purchased from Sigma Aldrich. D-amphetamine HCl is prepared in 0.9% saline to a concentration of 1.5 mg/ml. Salt form correction is not used in accordance with historical literature. D-Amphetamine was given I.P. per body weight at a dose of 1 ml/kg (=1.5 mg/kg). D-Amphetamine is prepared fresh from solid form 30 min. prior to each test period.

Dosing formulations of prodrug derivatives of antipsychotic parent drugs: Dosing solutions of paliperidone, risperidone, iloperidone, lurasidone, perospirone, and ziprasidone prodrugs of the invention useful in the treatment of schizophrenia and biopolar disorder are prepared. Dosing formulations comprise any number of suitable excipients for injection including but not limited to, i) oil emulsion in water with any combination of diphosphotidylcholine (DPPC), glycersol and NaOH, ii) aqueous suspensions including crystalline suspensions in any combination of hydroxypropylmethyl cellulose (HPMC) glycerol, phosphate buffered saline (PBS) and polysorbate (e.g. Tween 20).

Behavior Box: The behavior chambers are purchased from Med Associates, Inc. of St. Albans, Vt., Model ENV-515. Software for measuring animal movement is provided with the behavior chamber by the supplier.

Methods: The animals are acclimated for one week prior to commencing experimentation. The animals are initially acclimated to the behavior box for about 15 minutes before they are removed from the box and each dosed PO with 1.5 ml of one of paliperidone, risperidone, iloperidone, lurasidone, perospirone, or ziprasidone prodrug compounds of the invention, at concentrations which produce target therapeutic levels for paliperidone, risperidone, iloperidone, lurasidone, perospirone, or ziprasidone approximately 1 hour after administration. After an additional 15 minutes the animals are placed back in the behavior box for an additional 30 minute drug-baseline test session. The mice are then administered by IP injection, D-AMPH (1.5 mg/kg) followed by a 60 minute experimental behavioral measurement period. The parameters that are measured include a) total distance measured (primary measure), b) total number of ambulatory moves (second measure), c) total number of vertical moves (secondary measure) and d) time spent immobile (secondary measure.

Blood Sampling: Tail vein blood is taken on experiment days immediately following locomotor activity measurements (2-hours post-prodrug administration) and again the following day at time-points corresponding to 22 hours post-prodrug administration. Blood samples are collected via a lateral tail vein after anesthesia with Isoflurane. A 27½ G syringe without an anticoagulant is used for the blood collection, and the whole blood is transferred to pre-chilled (wet ice) tubes containing K2 EDTA. 0.5 ml of blood per animal is collected per time point. The tubes are inverted 15-20 times and immediately returned to the wet ice until being centrifuged for 2 minutes ≧14,000 g to separate plasma. The plasma samples that are prepared in this manner are transferred to labeled plain tubes (MICROTAINER®) and stored frozen at <−70° C.

Behavioral Data Acquisition: Behavioral data is captured electronically by the software package associated with the behavior chambers. Data is transformed and analyzed via GraphPad PRISM® 5 software (GraphPad Software, Inc., La Jolla, Calif.). The data is analyzed using a 2-way repeated measures ANOVA.

The patent and scientific literature referred to herein establishes the knowledge that is available to those with skill in the art. All United States patents and published or unpublished United States patent applications cited herein are incorporated by reference.

All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. What is claimed is a compound of formula I or II: 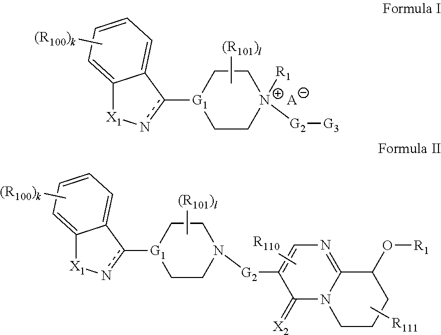
wherein [symbol] represents a single or double bond;
each k and l is independently 0, 1, 2, 3, or 4;
A⁻ is a pharmaceutically acceptable anion;
X₁ is —CR₁₀—, —O— or —S—;
wherein each R₁₀ is independently hydrogen, halogen, aliphatic, substituted aliphatic, aryl or substituted aryl;
X₂ is O or S;
G₁ is —N— or —CR₁₀—;
G₂ is selected from absent, —C(O)(C(R₁₀)(R₁₁))ₜ—, —C(R₁₀)═C(R₁₁)—, —(C(R₁₀)(R₁₁))ₐ═(C(R₁₀)(R₁₁)_{b}—, —(C(R₁₀)(R₁₁))ₐ—X₁₀—(C(R₁₀)(R₁₁)_{b}—, and —(C(R₁₀)(R₁₁))ₜ—;
wherein t is 1, 2, 3, 4, 5 or 6;
each a and b is independently 0, 1, 2, 3, 4, 5, 6, 7, 8, 9 or 10;
each R₁₁ is independently hydrogen, halogen, aliphatic, substituted aliphatic, aryl or substituted aryl;
X₁₀ is absent, cycloalkyl, —S—, —O—, —N(R₁₀)—, —C(O)—, —C(S)—, —C(R₁₀)═C(R₁₀)—, or —C≡C—;
alternatively two R₁₀ and R₁₁ groups together with the atoms they are attached form a three, four, five or six membered ring;
G₃ is an optionally substituted cyloalkyl or optionally substituted heterocyclyl;
R₁ is selected from —C(R₁₀)(R₁₁)—OR₁₂, —C(R₁₀)(R₁₁)—OC(O)OR₂₁, —C(R₁₀)(R₁₁)—OC(O)R₂₁, —C(R₁₀)(R₁₁)—OC(O)NR₁₂R₂₁, —C(R₁₀)(R₁₁)—OPO₃²⁻MY, —C(R₁₀)(R₁₁)—OP(O)(O⁻M)(OR₂₁), —C(R₁₀)(R₁₁)—OP(O)(OR₂₁)(OR₂₂);
each R₁₂ is independently hydrogen, halogen, aliphatic, substituted aliphatic, aryl or substituted aryl;
each R₂₁ and R₂₂ is independently hydrogen, aliphatic, substituted aliphatic, aryl or substituted aryl;
each R₁₀₀, R₁₀₁, R₁₁₀ and R₁₁₁ is independently selected from hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ alkylamino and optionally substituted C₁-C₈ aryl; and,
Y and M are the same or different and each is a monovalent cation;
or M and Y together is a divalent cation; and
optionally, the prodrug further comprises a biocompatible delivery system for delivering the prodrug wherein the system is capable of moderating accelerated hydrolytic cleavage of the prodrug by minimizing exposure of the prodrug to water or pH conditions deviating from the physiological range of pH.

2. The compound of claim 1 wherein G₃ is selected from: 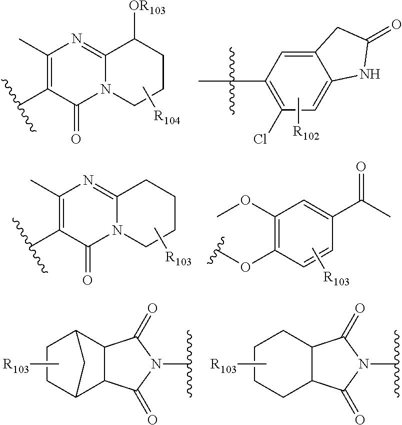
wherein R₁₀₂, R₁₀₃ and R₁₀₄ are independently selected from hydrogen, halogen, optionally substituted C₁-C₈ alkyl, optionally substituted C₂-C₈ alkenyl, optionally substituted C₂-C₈ alkynyl, optionally substituted C₃-C₈ cycloalkyl, optionally substituted C₁-C₈ alkoxy, optionally substituted C₁-C₈ alkylamino and optionally substituted C₁-C₈ aryl.

3. The compound of claim 1 wherein R₁ is selected from: 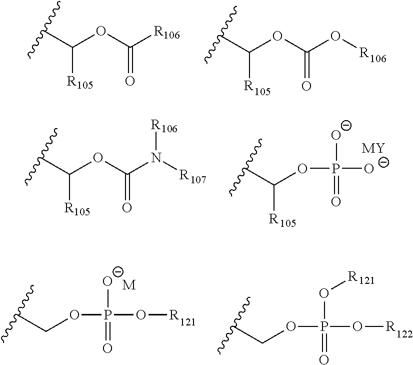
wherein R₁₀₅, R₁₀₆ and R₁₀₇ are independently selected from hydrogen, halogen, optionally substituted C₁-C₂₄ alkyl, optionally substituted C₂-C₂₄ alkenyl, optionally substituted C₂-C₂₄ alkynyl, optionally substituted C₃-C₂₄ cycloalkyl, optionally substituted C₁-C₂₄ alkoxy, optionally substituted C₁-C₂₄ alkylamino and optionally substituted C₁-C₂₄ aryl; and,
each R₁₂₁ and R₁₂₂ is independently hydrogen, aliphatic, substituted aliphatic, aryl or substituted aryl.

4. A compound of claim 1 wherein R₁ is selected from: 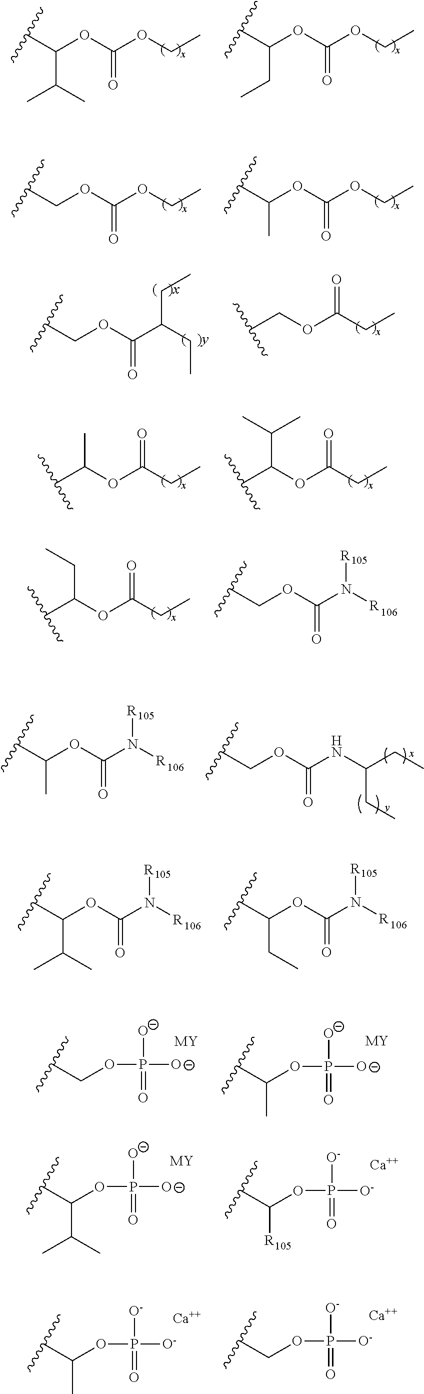 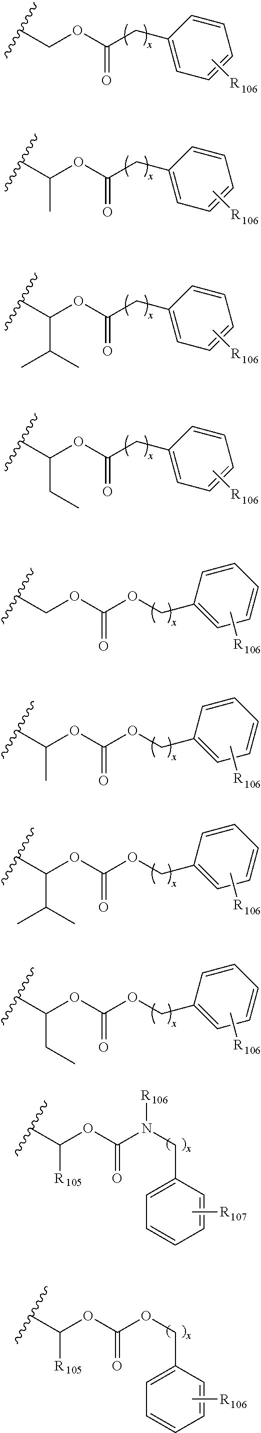 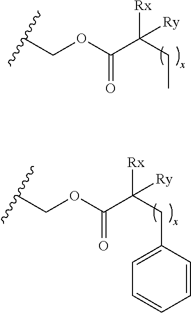
wherein x is an integer between 0 and 30;
each Rx and Ry is independently selected from hydrogen, halogen, optionally substituted alkyl, or taken together with the carbon to which they are attached form a C₃-C₈ cycloalkyl; and,
M, Y, R₁₀₅, R₁₀₆ and R₁₀₇ are as defined above.

5. A compound of claim 1 wherein R₁ selected from: 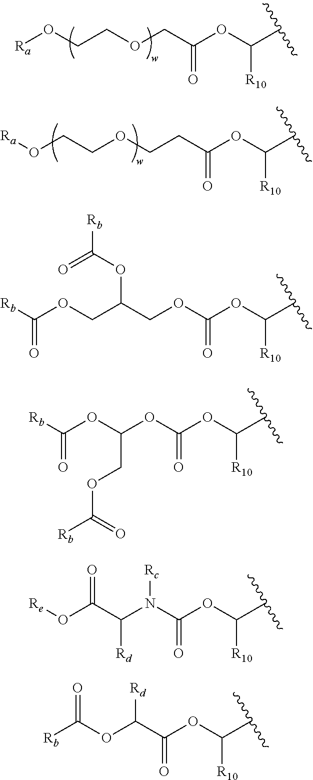
where w is 1 to about 1000; Rₐ, R_{b} and R_{c} are each independently C₁-C₂₄-alkyl, substituted C₁-C₂₄-alkyl, C₂-C₂₄-alkenyl, substituted C₂-C₂₄-alkenyl, C₂-C₂₄-alkynyl, substituted C₂-C₂₄-alkynyl, C₃-C₁₂-cycloalkyl, substituted C₃-C₁₂-cycloalkyl, aryl or substituted aryl; R_{c} is H or substituted or unsubstituted C₁-C₆-alkyl; R_{d} is H, substituted or unsubstituted C₁-C₆-alkyl, substituted or unsubstituted aryl-C₁-C₆-alkyl or substituted or unsubstituted heteroaryl-C₁-C₆-alkyl; R₁₀ is as defined above; alternatively R_{c} and R_{d} together with the carbon and nitrogen atoms to which they are attached, form a heterocycloalkyl group.

6. A compound of claim 1 wherein R₁ is selected from Table 1, 2, 3, 4, or 5.

7. A compound of claim 1 selected from Table A.

8. A compound according to claim 1 having the formula: 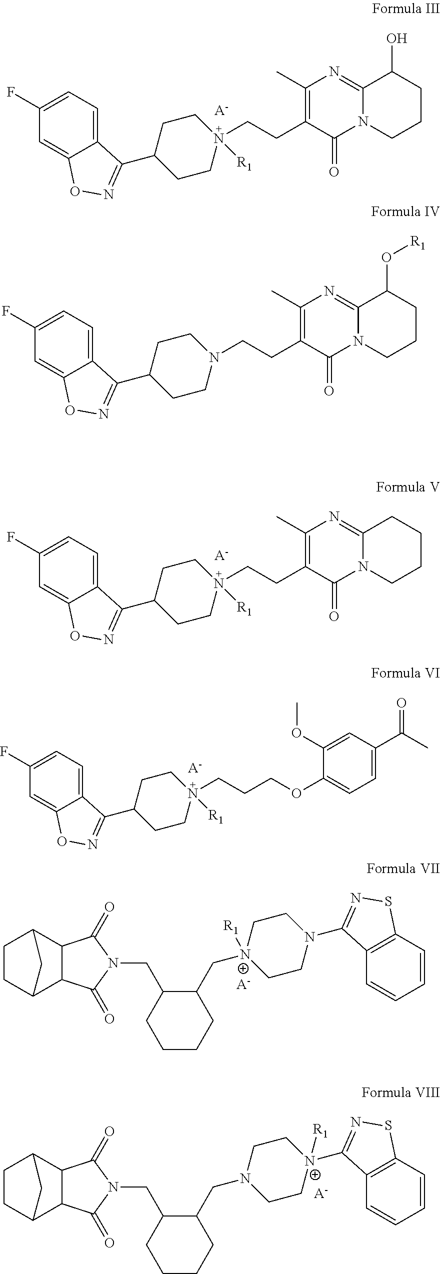 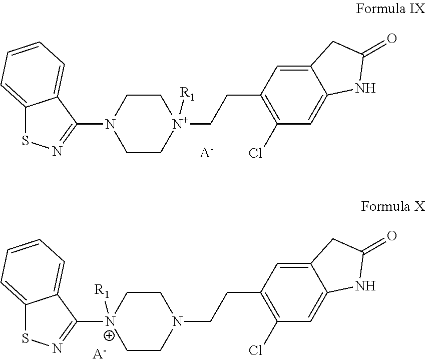
wherein, R₁ and A⁻ are as defined above.

9. A method of pH-independent sustained release delivery of a parent drug to a patient comprising administering a compound according to claim 1 to a patient.

10. A method of treating a neurological or psychiatric disorder by administering a compound according to claim 1 to a patient in need thereof.

11. A method according to claim 10, wherein said disorder is schizophrenia.

12. A method according to claim 10, wherein said disorder bipolar I disorder.

13. A method for the synthesis of a compound of formula I: 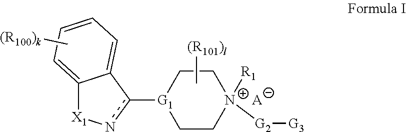
comprising the step of reacting a compound of formula XI, with a compound of the formula R₁—V, 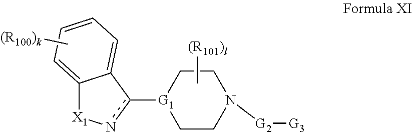
wherein k, l, R₁, X₁, R₁₀₀, R₁₀₁, G₁, G₂ and G₃ are as defined above; and,
V is a leaving group.

14. The method according to claim 13, wherein V is selected from iodine, bromine, chlorine, hydroxynaphthoate, naphthalenedisulfonate, tosylate, triflate and mesylate.

15. The method according to claim 13, wherein said reaction is performed in a polar aprotric solvent.

16. A method for sustained delivery of a parent drug of formula XI to a patient comprising administering a prodrug compound of the parent drug having the formula I wherein the prodrug compound has lower aqueous solubility at a reference pH as compared to the aqueous solubility of the parent drug at the same reference pH wherein the reference pH is a pH at which the parent drug is fully protonated and wherein upon administration to the patient, release of the parent drug from the prodrug is sustained release.

17. A method for pH-independent sustained delivery of a parent drug of formula XI to a patient comprising administering a prodrug compound of the parent drug having the formula I wherein upon administration of said prodrug said sustained release of parent drug is substantially pH independent.

18. A method for reducing sedation in a patient compared to administration of a parent drug of formula XI comprising administering a prodrug compound of the parent drug having the formula I wherein upon administration of said prodrug, dose dumping of the drug is reduced or eliminated.

19. A compound according to any of the above claims having formula: 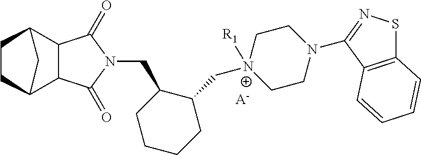
wherein R₁ and A− are as defined above.
